(19) United States
(12) Patent Application Publication
(10) Pub. No.: US 2024/0254217 A1
(43) Pub. Date: Aug. 1, 2024

(54) ANTI -IL-23P19 ANTIBODY REGULATION OF GENES INVOLVED IN ULCERATIVE COLITIS

(71) Applicant: ELI LILLY AND COMPANY, Indianapolis, IN (US)

(72) Inventors: Venkatesh KRISHNAN, Fishers, IN (US); Boyd Allen STEERE, Carmel, IN (US)

(21) Appl. No.: 18/563,387

(22) PCT Filed: May 27, 2022
(86) PCT No.: PCT/US2022/031328

§ 371 Date: Nov. 22, 2023

Related U.S. Application Data
(60) Provisional application No. 63/194,790, filed on May 28, 2021.
(60) Provisional application No. 63/295,636, filed on Dec. 31, 2021.

(51) Int. Cl. C07K 16/24; A61K 9/00; A61P 1/04; C12Q 1/6851; C12Q 1/6883

(52) U.S. Cl. CPC C07K 16/244; A61K 9/0019; A61P 1/04; C12Q 1/6851; C12Q 1/6883; C07K 2317/24; C07K 2317/76; C12Q 2600/106; C12Q 2600/158

(57) ABSTRACT

The present disclosure generally relates to methods of treating and diagnosing ulcerative colitis. The methods are particularly suitable for treating and diagnosing a specific sub-group of patients with ulcerative colitis. The methods are also particularly suitable for treating and diagnosing urgency in a patient having or suspected of having ulcerative colitis. The methods are also particularly suitable for treating and diagnosing stool frequency and bowel urgency in a patient having or suspected of having ulcerative colitis.

### BACKGROUND OF THE DISCLOSURE

The present disclosure relates generally to medicine. More particularly, the present disclosure relates to methods of treating and diagnosing ulcerative colitis. The methods are particularly suitable for treating and diagnosing a specific sub-group of patients having or suspected of having ulcerative colitis. The methods are also particularly suitable for treating and diagnosing urgency in a patient having or suspected of having ulcerative colitis. The methods are also particularly suitable for treating and diagnosing stool frequency and bowel urgency in a patient having or suspected of having ulcerative colitis.

Ulcerative colitis (UC) is a chronic relapsing immune-mediated inflammatory bowel disease (IBD) characterized by mucosal inflammation of the colon. Substantial morbidity and impaired quality of life results from typical symptoms such diarrhea, rectal bleeding, and urgency. Treatment aims include achieving symptom control (clinical remission), suppressing intestinal inflammation leading to mucosal healing (endoscopic remission), and preserving gut functionality. Current treatment options include 5-aminosalicylates, glucocorticoids, thiopurines, the Janus-associated kinase (JAK) inhibitor tofacitinib, and biologics that antagonize TNFα, the p-40 subunit of IL-12/IL-23, and a4b7 integrin. However, up to one third of patients do not respond to induction treatment and approximately 40% of patients who initially benefited subsequently lose response. It was recently shown that anti-TNF therapy is associated with potentially serious adverse effects. A new class of biologics that block integrin signaling, thereby reducing lymphocyte trafficking to the intestinal mucosa and reducing mucosal inflammation, represent a more favorable safety profile. In a recent trial vedolizumab, an a4p7 integrin blocker, was shown to be more effective than the TNF inhibitor adalimumab for moderate-to-severe UC.

Interleukin-23 (IL-23) is a novel therapeutic target in IBD, a heterodimeric cytokine composed of a p19 subunit and a p40 subunit that it shares with IL-12. IL-23 receptor engagement leads to activation of JAKs (mainly TYK2 and JAK2) and signal transducer and activator of transcription 3 and 4 (STAT3 and STAT4), triggering transcription of downstream target genes. IL-23 promotes the differentiation, maintenance and stabilization of pathogenic T-cell lineages, including populations that simultaneously produce multiple pro-inflammatory cytokines, such as interferon-y, IL-17A, IL-17F and IL-22, as well as activation and induction of effector function of colitogenic innate lymphoid cells. Therapeutic blockade of p40 is effective in both UC and CD, and drugs targeting p19 are being studied for both UC and CD.

Data from single cell RNASeq studies have suggested that inflamed mucosal fibroblasts, tissue resident monocytes, and dendritic cells are enriched in anti-TNF-resistant (TNF^{R}) therapy compared to UC patients who respond to anti-TNFs. In these studies, Smillie et al. scored cell subsets for gene signatures of TNF^{R} and sensitivity based on a meta-analysis of bulk expression data from 60 responders and 57 non-responders to anti-TNF therapy. TNF^{R} was strongly associated with genes enriched in immune associated fibroblasts (IAFs), inflammatory monocytes, and DC2 cells. In contrast, favourable response to anti-TNF therapy was evident in the transcriptome signature in epithelial cells, which represents healthy mucosa prevalent in UC patients in remission.

There remains a need for alternative compositions and methods to diagnose and treat inflammatory bowel diseases such as ulcerative colitis.

### BRIEF DESCRIPTION OF THE DISCLOSURE

The present disclosure is generally relates to methods of treating and diagnosing ulcerative colitis. The methods are particularly suitable for treating and diagnosing a specific sub-group of patients with ulcerative colitis. The methods are also particularly suitable for treating and diagnosing urgency in a patient having ulcerative colitis. The methods are also particularly suitable for treating and diagnosing stool frequency and bowel urgency in a patient having ulcerative colitis.

The present inventors have determined that the expression of a number of genes (as determined by measuring changes in gene transcript biomarkers in colon or rectal tissue samples) occur in response to treatment of patients having, or suspected as having, ulcerative colitis with an anti-IL-23p19 antibody. The gene transcripts may be used as biomarkers to diagnose ulcerative colitis, symptoms of ulcerative colitis, stool frequency associated with ulcerative colitis and bowel urgency associated with ulcerative colitis. The gene transcripts may also be used as biomarkers of a successful response to treatment with an anti-IL-23p19 antibody.

Accordingly, in a first aspect of the present invention there is provided a method of treating ulcerative colitis in a patient having or suspected of having ulcerative colitis, wherein the method comprises:
obtaining a first sample from the patient; analyzing the first sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16;
administering an anti-IL-23p19 antibody to the patient;
obtaining a second sample from the patient; and analyzing the second sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16,
wherein a change in expression level of the one or more gene transcript biomarker(s) detected in the second sample from the expression level of the one or more gene transcript biomarkers detected in the first sample indicates a response to the anti-IL-23p19 antibody.

In a further aspect of the present invention, there is provided an anti-IL-23p19 antibody for use in the treatment of ulcerative colitis, wherein the treatment comprises:
obtaining a first sample from a patient having, or suspected of having, ulcerative colitis;
analyzing the first sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16;
administering the anti-IL-23p19 antibody to the patient;
obtaining a second sample from the patient; and analyzing the second sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16, wherein a change in expression level of the one or more gene transcript biomarker(s) detected in the second sample from the expression level of the one or more gene transcript biomarkers detected in the first sample indicates a response to the anti-IL-23p19 antibody.

In a still further aspect of the present invention, there is provided the use of an anti-IL-23p19 antibody in the manufacture of a medicament for use in the treatment of ulcerative colitis, wherein the treatment comprises:
obtaining a first sample from a patient having, or suspected of having, ulcerative colitis;
analyzing the first sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16;
administering the anti-IL-23p19 antibody to the patient;
obtaining a second sample from the patient; and analyzing the second sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16,
wherein a change in expression level of the one or more gene transcript biomarker(s) detected in the second sample from the expression level of the one or more gene transcript biomarkers detected in the first sample indicates a response to the anti-IL-23p19 antibody.

In a further aspect of the present invention, there is provided a method of treating ulcerative colitis in a patient having or suspected of having ulcerative colitis, said method comprising:
obtaining a first sample from the patient;
analyzing the first sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2 and ADH1C;
administering an anti-IL-23p19 antibody to the patient;
obtaining a second sample from the patient; and
analyzing the second sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2 and ADH1C;
wherein a change in expression level of the one or more gene transcript biomarker(s) detected in the second sample from the expression level of the one or more gene transcript biomarkers detected in the first sample indicates a response to the anti-IL-23p19 antibody.

In a still further aspect of the present invention, there is provided an anti-IL-23p19 antibody for use in the treatment of ulcerative colitis, wherein the treatment comprises:
obtaining a first sample from a patient having, or suspected of having, ulcerative colitis;
analyzing the first sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2 and ADH1C;
administering an anti-IL-23p19 antibody to the patient;
obtaining a second sample from the patient; and
analyzing the second sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2 and ADH1C;
wherein a change in expression level of the one or more gene transcript biomarker(s) detected in the second sample from the expression level of the one or more gene transcript biomarkers detected in the first sample indicates a response to the anti-IL-23p19 antibody.

In a still further aspect of the present invention, there is provided the use of an anti-IL-23p19 antibody for the manufacture of a medicament for use in the treatment of ulcerative colitis, said treatment comprising:
obtaining a first sample from a patient having, or suspected of having, ulcerative colitis;
analyzing the first sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2 and ADH1C;
administering an anti-IL-23p19 antibody to the patient;
obtaining a second sample from the patient; and
analyzing the second sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2 and ADH1C;
wherein a change in expression level of the one or more gene transcript biomarker(s) detected in the second sample from the expression level of the one or more gene transcript biomarkers detected in the first sample indicates a response to the anti-IL-23p19 antibody.

In a still further aspect of the present invention, there is provided a method of treating a symptom associated with ulcerative colitis in a patient having or suspected of having ulcerative colitis, wherein the method comprises:
obtaining a first sample from the patient;
analyzing the first sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16;
administering an anti-IL-23p19 antibody to the patient;
obtaining a second sample from the patient; and analyzing the second sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16,
wherein a change in expression level of the one or more gene transcript biomarker(s) detected in the second sample from the expression level of the one or more gene transcript biomarkers detected in the first sample indicates a response to the anti-IL-23p19 antibody.

In a still further aspect of the present invention, there is provided an anti-IL-23p19 antibody for use in the treatment of a symptom associated with ulcerative colitis, wherein the treatment comprises:
obtaining a first sample from the patient having, or suspected of having, ulcerative colitis;
analyzing the first sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16;
administering an anti-IL-23p19 antibody to the patient;
obtaining a second sample from the patient; and analyzing the second sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16,
wherein a change in expression level of the one or more gene transcript biomarker(s) detected in the second sample from the expression level of the one or more gene transcript biomarkers detected in the first sample indicates a response to the anti-IL-23p19 antibody.

In a still further aspect of the present invention, there is provided the use of an anti-IL-23p19 antibody for the manufacture of a medicament for use in the treatment of a symptom associated with ulcerative colitis, wherein the treatment comprises:
obtaining a first sample from the patient having, or suspected of having, ulcerative colitis;
analyzing the first sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16;
administering an anti-IL-23p19 antibody to the patient;
obtaining a second sample from the patient; and analyzing the second sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16,
wherein a change in expression level of the one or more gene transcript biomarker(s) detected in the second sample from the expression level of the one or more gene transcript biomarkers detected in the first sample indicates a response to the anti-IL-23p19 antibody.

In a still further aspect of the present invention, there is provided a method of treating a symptom associated with ulcerative colitis in a patient having or suspected of having ulcerative colitis, wherein the method comprises:
obtaining a first sample from the patient;
analyzing the first sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2 and ADH1C;
administering an anti-IL-23p19 antibody to the patient;
obtaining a second sample from the patient; and analyzing the second sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, and ADH1C;
wherein a change in expression level of the one or more gene transcript biomarker(s) detected in the second sample from the expression level of the one or more gene transcript biomarkers detected in the first sample indicates a response to the anti-IL-23p19 antibody.

In a still further aspect of the present invention, there is provided an anti-IL-23p19 antibody for use in the treatment of a symptom associated with ulcerative colitis, wherein the treatment comprises:
obtaining a first sample from a patient having, or suspected of having, ulcerative colitis;
analyzing the first sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2 and ADH1C;
administering an anti-IL-23p19 antibody to the patient;
obtaining a second sample from the patient; and analyzing the second sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, and ADH1C;
wherein a change in expression level of the one or more gene transcript biomarker(s) detected in the second sample from the expression level of the one or more gene transcript biomarkers detected in the first sample indicates a response to the anti-IL-23p19 antibody.

In a still further aspect of the present invention, there is provided the use of an anti-IL-23p19 antibody in the manufacture of a medicament for use in the treatment of a symptom associated with ulcerative colitis, wherein the treatment comprises:
obtaining a first sample from a patient having, or suspected of having, ulcerative colitis;
analyzing the first sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2 and ADH1C;
administering an anti-IL-23p19 antibody to the patient;
obtaining a second sample from the patient; and analyzing the second sample to detect one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, and ADH1C;
wherein a change in expression level of the one or more gene transcript biomarker(s) detected in the second sample from the expression level of the one or more gene transcript biomarkers detected in the first sample indicates a response to the anti-IL-23p19 antibody.

Preferably, the symptom is one or more of abdominal pain/discomfort, blood in stool, pus in stool, fever, weight loss, rectal bleeding, frequent diarrhea, recurrent diarrhea, fatigue, reduced appetite, and tenesmus (urgency).

In a still further aspect of the present invention, there is provided a method of treating a patient having or suspected of having ulcerative colitis and who has or is suspected of having anti-Tumor Necrosis Factor (anti-TNF) therapy resistance (anti-TNF^{R}) with an anti-IL-23p19 antibody, wherein the method comprises:
determining if the patient is anti-TNF^{R} by obtaining a sample from the patient and analyzing the sample for one or more anti-Tumor Necrosis Factor (anti-TNF) therapy resistance (anti-TNF^{R}) gene transcript biomarker(s) of one or more genes selected from OSMR, FCGR3, CXCL6, interleukin-11, interleukin-24, interleukin-13RA2, FAP, TWIST1, and WNT2,
and treating the patient with an anti-IL-23p19 antibody if the patient is anti-TNF^{R}.

In a still further aspect of the present invention, there is provided an anti-IL-23p19 antibody for use in the treatment of ulcerative colitis, wherein the treatment comprises:
obtaining a sample from a patient having or suspected of having ulcerative colitis and who has or is suspected of having anti-Tumor Necrosis Factor (anti-TNF) therapy resistance (anti-TNF^{R}) to determine if the patient is anti-TNF^{R}; and
analyzing the sample for one or more anti-Tumor Necrosis Factor (anti-TNF) therapy resistance (anti-TNF^{R}) gene transcript biomarker(s) of one or more genes selected from OSMR, FCGR3, CXCL6, interleukin-11, interleukin-24, interleukin-13RA2, FAP, TWIST1, and WNT2,
and treating the patient with an anti-IL-23p19 antibody if the patient is anti-TNF^{R}.

In a still further aspect of the present invention, there is provided the use of an anti-IL-23p19 antibody for the manufacture of a medicament for use in the treatment of ulcerative colitis, wherein the treatment comprises:
obtaining a sample from a patient having or suspected of having ulcerative colitis and who has or is suspected of having anti-Tumor Necrosis Factor (anti-TNF) therapy resistance (anti-TNF^{R}) to determine if the patient is anti-TNF^{R}; and
analyzing the sample for one or more anti-Tumor Necrosis Factor (anti-TNF) therapy resistance (anti-TNF^{R}) gene transcript biomarker(s) of one or more genes selected from OSMR, FCGR3, CXCL6, interleukin-11, interleukin-24, interleukin-13RA2, FAP, TWIST1, and WNT2,
and treating the patient with an anti-IL-23p19 antibody if the patient is anti-TNF^{R}.

Preferably, the method or treatment further comprises analyzing samples obtained before anti-IL-23p19 antibody administration and following anti-IL-23p19 antibody administration for one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16,
wherein a change in expression level of the one or more gene transcript biomarker(s) detected in the sample obtained after administration of the anti-IL-23p19 antibody from the expression level of the one or more gene transcript biomarkers detected in the sample obtained prior to administration of the anti-IL-23p19 antibody indicates a response to the anti-IL-23p19 antibody in the anti-TNF^{R} patient.

In a still further aspect of the present invention, there is provided a method of treating stool frequency in a patient having or suspected of having ulcerative colitis, wherein the method comprises:
obtaining a first sample from the patient;
analyzing the first sample to detect one or more gene transcript biomarker(s) of genes selected from Table 8;
administering an anti-IL-23p19 antibody to the patient;
obtaining a second sample from the patient; and
analyzing the second sample to detect one or more gene transcript biomarker(s) of one or more genes selected from above,
wherein a change in expression level of the one or more gene transcript biomarker(s) detected in the second sample from the expression level of the one or more gene transcript biomarker(s) detected in the first sample indicates a response to the anti-IL-23p19 antibody.

In a still further aspect of the present invention, there is provided an anti-IL-23p19 antibody for use in the treatment of stool frequency associated with ulcerative colitis, wherein the treatment comprises:
obtaining a first sample from a patient having, or suspected of having, ulcerative colitis;
analyzing the first sample to detect one or more gene transcript biomarker(s) of genes selected from Table 8;
administering an anti-IL-23p19 antibody to the patient;
obtaining a second sample from the patient; and
analyzing the second sample to detect one or more gene transcript biomarker(s) of one or more genes selected from Table 8,
wherein a change in expression level of the one or more gene transcript biomarker(s) detected in the second sample from the expression level of the one or more gene transcript biomarker(s) detected in the first sample indicates a response to the anti-IL-23p19 antibody.

In a still further aspect of the present invention, there is provided the use of anti-IL-23p19 antibody for the manufacture of a medicament for use in the treatment of stool frequency associated with ulcerative colitis, wherein the treatment comprises:
obtaining a first sample from a patient having, or suspected of having, ulcerative colitis;
analyzing the first sample to detect one or more gene transcript biomarker(s) of genes selected from Table 8;
administering an anti-IL-23p19 antibody to the patient;
obtaining a second sample from the patient; and
analyzing the second sample to detect one or more gene transcript biomarker(s) of one or more genes selected from Table 8,
wherein a change in expression level of the one or more gene transcript biomarker(s) detected in the second sample from the expression level of the one or more gene transcript biomarker(s) detected in the first sample indicates a response to the anti-IL-23p19 antibody.

In a still further aspect of the present invention, there is provided a method of treating stool frequency in a patient having or suspected of having ulcerative colitis, wherein the method comprises:
obtaining a first sample from the patient;
analyzing the first sample to detect one or more gene transcript biomarker(s) of genes selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, and Creatine kinase B,
administering an anti-IL-23p19 antibody to the patient;
obtaining a second sample from the patient; and
analyzing the second sample to detect one or more gene transcript biomarker(s) of one or more genes selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, and Creatine kinase B,
wherein a change in expression level of the one or more gene transcript biomarker(s) detected in the second sample from the expression level of the one or more gene transcript biomarker(s) detected in the first sample indicates a response to the anti-IL-23p19 antibody.

In a still further aspect of the present invention, there is provided an anti-IL-23p19 antibody for use in the treatment in of stool frequency associated with ulcerative colitis, wherein the treatment comprises:
obtaining a first sample from a patient having, or suspected of having, ulcerative colitis;
analyzing the first sample to detect one or more gene transcript biomarker(s) of genes selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, and Creatine kinase B,
administering an anti-IL-23p19 antibody to the patient;
obtaining a second sample from the patient; and
analyzing the second sample to detect one or more gene transcript biomarker(s) of one or more genes selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, and Creatine kinase B,
wherein a change in expression level of the one or more gene transcript biomarker(s) detected in the second sample from the expression level of the one or more gene transcript biomarker(s) detected in the first sample indicates a response to the anti-IL-23p19 antibody.

In a still further aspect of the present invention, there is provided an anti-IL-23p19 antibody for use in the treatment in of stool frequency associated with ulcerative colitis, wherein the treatment comprises:
obtaining a first sample from a patient having, or suspected of having, ulcerative colitis;
analyzing the first sample to detect one or more gene transcript biomarker(s) of genes selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, and Creatine kinase B,
administering an anti-IL-23p19 antibody to the patient;
obtaining a second sample from the patient; and
analyzing the second sample to detect one or more gene transcript biomarker(s) of one or more genes selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, and Creatine kinase B,
wherein a change in expression level of the one or more gene transcript biomarker(s) detected in the second sample from the expression level of the one or more gene transcript biomarker(s) detected in the first sample indicates a response to the anti-IL-23p19 antibody.

In a still further aspect of the present invention, there is provided the use of an anti-IL-23p19 antibody for the manufacture of a medicament for use in the treatment in of stool frequency associated with ulcerative colitis, wherein the treatment comprises:
obtaining a first sample from a patient having, or suspected of having, ulcerative colitis;
analyzing the first sample to detect one or more gene transcript biomarker(s) of genes selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, and Creatine kinase B,
administering an anti-IL-23p19 antibody to the patient;
obtaining a second sample from the patient; and
analyzing the second sample to detect one or more gene transcript biomarker(s) of one or more genes selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, and Creatine kinase B,
wherein a change in expression level of the one or more gene transcript biomarker(s) detected in the second sample from the expression level of the one or more gene transcript biomarker(s) detected in the first sample indicates a response to the anti-IL-23p19 antibody.

In a still further aspect of the present invention, there is provided a method of treating bowel urgency in a patient having or suspected of having ulcerative colitis, the method comprising:
obtaining a first sample from the patient;
analyzing the first sample to detect one or more gene transcript biomarker(s) of one or more genes selected from Table 9;
administering an anti-IL-23p19 antibody to the patient;
obtaining a second sample from the patient;
analyzing the second sample to detect one or more gene transcript biomarker(s) of one or more genes from Table 9;
wherein a change in expression level of the one or more gene transcript biomarker(s) detected in the second sample from the expression level of the one or more gene transcript biomarker(s) detected in the first sample indicates a response to the anti-IL-23p19 antibody.

In a still further aspect of the present invention, there is provided an anti-IL-23p19 antibody for use in the treatment in of bowel urgency associated with ulcerative colitis, wherein the treatment comprises:
obtaining a first sample from a patient having, or suspected of having, ulcerative colitis;
analyzing the first sample to detect one or more gene transcript biomarker(s) of one or more genes selected from Table 9;
administering an anti-IL-23p19 antibody to the patient;
obtaining a second sample from the patient;
analyzing the second sample to detect one or more gene transcript biomarker(s) of one or more genes from Table 9;
wherein a change in expression level of the one or more gene transcript biomarker(s) detected in the second sample from the expression level of the one or more gene transcript biomarker(s) detected in the first sample indicates a response to the anti-IL-23p19 antibody.

In a still further aspect of the present invention, there is provided the use of anti-IL-23p19 antibody for the manufacture of a medicament for use in the treatment of bowel urgency associated with ulcerative colitis, wherein the treatment comprises:
obtaining a first sample from a patient having, or suspected of having, ulcerative colitis;
analyzing the first sample to detect one or more gene transcript biomarker(s) of one or more genes selected from Table 9;
administering an anti-IL-23p19 antibody to the patient;
obtaining a second sample from the patient;
analyzing the second sample to detect one or more gene transcript biomarker(s) of one or more genes from Table 9;
wherein a change in expression level of the one or more gene transcript biomarker(s) detected in the second sample from the expression level of the one or more gene transcript biomarker(s) detected in the first sample indicates a response to the anti-IL-23p19 antibody.

In a still further aspect of the present invention, there is provided a method of treating bowel urgency in a patient having or suspected of having ulcerative colitis, the method comprising:
obtaining a first sample from the patient;
analyzing the first sample to detect one or more gene transcript biomarker(s) of one or more genes selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, and Calpain 13;
administering an anti-IL-23p19 antibody to the patient;
obtaining a second sample from the patient;
analyzing the second sample to detect one or more gene transcript biomarker(s) of one or more genes from one or more gene transcript biomarker(s) of one or more genes selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, and Calpain 13;
wherein a change in expression level of the one or more gene transcript biomarker(s) detected in the second sample from the expression level of the one or more gene transcript biomarker(s) detected in the first sample indicates a response to the anti-IL-23p19 antibody.

In a still further aspect of the present invention, there is provided an anti-IL-23p19 antibody for use in the treatment of bowel urgency associated with ulcerative colitis, wherein the treatment comprises:
obtaining a first sample from a patient having, or suspected of having, ulcerative colitis;
analyzing the first sample to detect one or more gene transcript biomarker(s) of one or more genes selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, and Calpain 13;
administering an anti-IL-23p19 antibody to the patient;
obtaining a second sample from the patient;
analyzing the second sample to detect one or more gene transcript biomarker(s) of one or more genes from one or more gene transcript biomarker(s) of one or more genes selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, and Calpain 13;
wherein a change in expression level of the one or more gene transcript biomarker(s) detected in the second sample from the expression level of the one or more gene transcript biomarker(s) detected in the first sample indicates a response to the anti-IL-23p19 antibody.

In a still further aspect of the present invention, there is provided the use of an anti-IL-23p19 antibody for the manufacture of a medicament for use in the treatment of bowel urgency associated with ulcerative colitis, wherein the treatment comprises:
obtaining a first sample from a patient having, or suspected of having, ulcerative colitis;
analyzing the first sample to detect one or more gene transcript biomarker(s) of one or more genes selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, and Calpain 13;
administering an anti-IL-23p19 antibody to the patient;
obtaining a second sample from the patient;
analyzing the second sample to detect one or more gene transcript biomarker(s) of one or more genes from one or more gene transcript biomarker(s) of one or more genes selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, and Calpain 13;
wherein a change in expression level of the one or more gene transcript biomarker(s) detected in the second sample from the expression level of the one or more gene transcript biomarker(s) detected in the first sample indicates a response to the anti-IL-23p19 antibody.

In a preferred aspect of the methods, treatments and uses of the present invention, the method, treatment or use comprises detecting the expression level of at least two gene transcript biomarkers of the aforementioned genes, where appropriate prior to and after administration of the anti-IL-23p19 antibody.

In a further preferred aspect of the methods, treatments and uses of the present invention, the method, treatment or use comprises detecting the expression level of at least three gene transcript biomarkers of the aforementioned genes, where appropriate prior to and after administration of the anti-IL-23p19 antibody.

In a still further preferred aspect of the methods, treatments and uses of the present invention, the method, treatment or use comprises detecting the expression level of at least four gene transcript biomarkers of the aforementioned genes, where appropriate prior to and after administration of the anti-IL-23p19 antibody.

In a still further preferred aspect of the methods, treatments and uses of the present invention, the method, treatment or use comprises detecting the expression level of at least five gene transcript biomarkers of the aforementioned genes, where appropriate prior to and after administration of the anti-IL-23p19 antibody.

In a still further preferred aspect of the methods, treatments and uses of the present invention, the method, treatment or use comprises detecting the expression level of at least six gene transcript biomarkers of the aforementioned genes, where appropriate prior to and after administration of the anti-IL-23p19 antibody.

In a still further preferred aspect of the methods, treatments and uses of the present invention, the method, treatment or use comprises detecting the expression level of at least seven gene transcript biomarkers of the aforementioned genes, where appropriate prior to and after administration of the anti-IL-23p19 antibody.

In a still further preferred aspect of the methods, treatments and uses of the present invention, the method, treatment or use comprises detecting the expression level of at least eight gene transcript biomarkers of the aforementioned genes, where appropriate prior to and after administration of the anti-IL-23p19 antibody.

In a still further preferred aspect of the methods, treatments and uses of the present invention, the method, treatment or use comprises detecting the expression level of at least nine gene transcript biomarkers of the aforementioned genes, where appropriate prior to and after administration of the anti-IL-23p19 antibody.

In a still further preferred aspect of the methods, treatments and uses of the present invention, the method, treatment or use comprises detecting the expression level of at least ten gene transcript biomarkers of the aforementioned genes, where appropriate prior to and after administration of the anti-IL-23p19 antibody.

In a still further preferred aspect of the methods, treatments and uses of the present invention, a change in the expression of the one or more gene transcript biomarkers detected in the second sample from the expression of the one or more gene transcript biomarkers detected in the first sample indicates that administration of the anti-IL-23p19 antibody should be continued.

In a still further preferred aspect of the methods, treatments and uses of the present invention, one or more gene transcript biomarker(s) is increased following administration of the anti-IL-23p19 antibody, and wherein the one or more gene transcript biomarker(s) are GUCA2B, OTOP2, AQP8, SLC26A2, ADH1C, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4 and ZG16.

In a still further preferred aspect of the methods, treatments and uses of the present invention, one or more gene transcript biomarker(s) is decreased following the anti-IL-23p19 antibody treatment, and wherein the one or more gene transcript biomarker(s) are CXCL8, AQP9, IL1B, S100A9, TREM1, MMP12, MMP1, MMP7, TCN1, DUOX2, DUOXA2, SLC6A14, VNN1, ABCA12, REG1B, C4BPA, REG3A, DMBT1, REG1P, S100A8, IGKV2D-40, PI3, TNIP3, REG1A, IDO1, NOS2, MMP10, CXCL1 and PTGS2.

In a still further preferred aspect of the methods, treatments and uses of the present invention, the expression level of the one of more gene transcript biomarker(s) is determined by a method of gene expression profiling.

In a still further preferred aspect of the methods, treatments and uses of the present invention, the method of gene expression profiling is a PCR-based method.

In a still further preferred aspect of the methods, treatments and uses of the present invention, the method of gene expression profiling is immunohistochemistry.

In a still further preferred aspect of the methods, treatments and uses of the present invention, the method of gene expression profiling is a proteomics technology.

In a still further preferred aspect of the methods, treatments and uses of the present invention, the expression levels of the one or more gene transcript biomarker(s) are normalized relative to the expression levels of one or more reference genes, or their expression products.

In a still further preferred aspect of the methods, treatments and uses of the present invention, the sample is from a colonic tissue biopsy or rectal tissue biopsy.

In a still further preferred aspect of the methods, treatments and uses of the present invention, the colonic tissue biopsy is from a tissue selected from the group consisting of the terminal ileum, the ascending colon, the descending colon, and the sigmoid colon.

In a still further preferred aspect of the methods, treatments and uses of the present invention, the colonic tissue biopsy is from a non-inflamed colonic area.

In a still further preferred aspect of the methods, treatments and uses of the present invention, the colonic tissue biopsy is from an inflamed colonic area.

In a still further preferred aspect of the methods, treatments and uses of the present invention, the first sample is taken before or simultaneous with administration of the anti-IL-23p19 antibody and wherein the second sample is taken at least two weeks, at least four weeks, at least eight weeks, at least twelve weeks, at least sixteen weeks, at least twenty weeks, at least twenty-four weeks, at least twenty-eight weeks, at least thirty weeks, at least thirty-two weeks, at least thirty-six weeks, at least forty weeks, at least forty-four weeks, at least forty-eight weeks, or at least fifty-two weeks, after the first administration of the anti-IL-23p19 antibody.

In a still further preferred aspect of the methods, treatments and uses of the present invention, the anti-IL-23p19 antibody is mirikizumab, guselkumab, risankizumab, tildrakizumab or brazikumab.

In a still further preferred aspect of the methods, treatments and uses of the present invention, the anti-IL-23p19 antibody is mirikizumab.

Preferably, the method, treatment or use comprises:
a) administering three induction doses of mirikizumab to the patient by intravenous infusion at 4-week intervals, wherein each induction dose comprises 300 mg of mirikizumab; and
b) administering maintenance doses of mirikizumab to the patient by subcutaneous injection at 4 week or 12 week intervals, wherein the first maintenance dose is administered 2-8 weeks after the last induction dose is administered and wherein each maintenance dose comprises 200 mg of mirikizumab.

Further preferably, the first maintenance dose of mirikizumab is administered 4-6 weeks after the last induction dose is administered.

Further preferably, subsequent maintenance dose(s) of mirikizumab are administered at 4-week intervals after administration of the first maintenance dose.

Alternatively preferably, subsequent maintenance dose(s) of mirikizumab are administered at 12-week intervals after administration of the first maintenance dose.

In a still further preferred aspect of the methods, treatments and uses of the present invention, the anti-IL-23p19 antibody is guselkumab.

Preferably, the method, treatment or use comprises:
a) administering three induction doses of guselkumab to the patient by intravenous infusion at 4-week intervals, wherein each induction dose comprises 100-500 mg of guselkumab; and
b) administering maintenance doses of guselkumab to the patient by subcutaneous injection at 2-week, 4-week, 6-week, 8-week or 12-week intervals, wherein the first maintenance dose is administered 2-8 weeks after the last induction dose is administered.

Further preferably, each induction dose comprises 200 mg of guselkumab.

Alternatively preferably, each induction dose comprises 400 mg of guselkumab.

In a still further preferred aspect of the methods, treatments and uses of the present invention, the anti-IL-23p19 antibody is risankizumab.

In a still further preferred aspect of the methods, treatments and uses of the present invention, the anti-IL-23p19 antibody is tildrakizumab.

In a still further preferred aspect of the methods, treatments and uses of the present invention, the anti-IL-23p19 antibody is brazikumab.

In a further aspect of the present invention, there is provided a method of identifying a patient having or suspected of having anti-Tumor Necrosis Factor (anti-TNF) therapy resistance (anti-TNF^{R}) as a candidate patient for receiving anti-IL-23p19 antibody treatment for ulcerative colitis, wherein the method comprises:
obtaining a sample from the patient;
analyzing the sample for one or more anti-Tumor Necrosis Factor (anti-TNF) therapy resistance (anti-TNF^{R}) gene transcript biomarker(s) of one or more genes selected from OSMR, FCGR3, CXCL6, interleukin-11, interleukin-24, interleukin-13RA2, FAP, TWIST1, and WNT2; and
identifying the patient as a candidate patient for receiving anti-IL-23p19 antibody treatment based on the analysis of the gene transcript biomarker.

Preferably, the method of identifying a patient having or suspected of having anti-Tumor Necrosis Factor (anti-TNF) therapy resistance (anti-TNF^{R}) as a candidate patient for receiving anti-IL-23p19 antibody treatment for ulcerative colitis further comprises analyzing the or another sample obtained from the patient for one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16.

In a further aspect of the present invention, there is provided a method for diagnosing ulcerative colitis in a patient having or suspected of having ulcerative colitis, wherein the method comprises:
determining an expression level of one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16 in a sample obtained from the patient, comparing the determined expression level of the one or more gene transcript biomarker(s) to a reference expression level of one or more gene transcript biomarker(s) selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; and
providing a diagnosis of ulcerative colitis if the biomarker expression level in the patient is increased as compared to the reference expression level or if the biomarker expression level in the patient is decreased as compared to the reference expression level.

In a still further aspect of the present invention, there is provided a method of determining whether a patient having or suspected of having ulcerative colitis is healing in response to treatment with an anti-IL-23p19 antibody, wherein the method comprises:
(a)(i) analyzing a sample obtained from a patient before the patient receives anti-IL-23p19 antibody treatment for one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16;
(b)(i) analyzing a sample obtained from a patient after the patient receives the anti-IL-23p19 antibody treatment for one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; and
(c)(i) determining that the patient having or suspected of having ulcerative colitis is healing in response to the anti-IL-23p19 antibody treatment if a change in expression level in the one or more gene transcript biomarker(s) after the patient receives the anti-IL-23p19 antibody treatment is detected or
(a)(ii) analyzing a sample obtained from a patient having or suspected of having ulcerative colitis who did not receive anti-IL-23p19 antibody treatment for one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16;
(b)(ii) analyzing a sample obtained from a patient after the patient receives the anti-IL-23p19 antibody treatment for one or more gene transcript biomarker(s) of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; and
(c)(ii) determining that the patient having or suspected of having ulcerative colitis is healing in response to the anti-IL-23p19 antibody treatment if a change in expression level in the one or more gene transcript biomarker(s) after the patient receives the anti-IL-23p19 antibody treatment is detected as compared to the expression level of the one or more gene transcript biomarker(s) in the patient who did not receive anti-IL-23p19 antibody treatment.

In a still further aspect of the present invention, there is provided a method of determining whether a patient having or suspected of having ulcerative colitis is healing in response to treatment with an anti-IL-23p19 antibody according to claim 151, wherein the method comprises:
(a)(i) analyzing a sample obtained from a patient before the patient receives anti-IL-23p19 antibody treatment for one or more gene transcript biomarker(s) of one or more genes selected from GUCA2A, OTOP2, AQP8, SLC26A2, and ADH1C,
(b)(i) analyzing a sample obtained from a patient after the patient receives the anti-IL-23p19 antibody treatment for one or more gene transcript biomarker(s) of one or more genes selected from GUCA2A, OTOP2, AQP8, SLC26A2, and ADH1C; and
(c)(i) determining that the patient having or suspected of having ulcerative colitis is healing in response to the anti-IL-23p19 antibody treatment if the expression level in the one or more gene transcript biomarker(s) is increased after the patient receives the anti-IL-23p19 antibody treatment or
(a)(ii) analyzing a sample obtained from a patient having or suspected of having ulcerative colitis who did not receive anti-IL-23p19 antibody treatment for one or more gene transcript biomarker(s) of one or more genes selected from GUCA2A, OTOP2, AQP8, SLC26A2, and ADH1C;
(b)(ii) analyzing a sample obtained from a patient after the patient receives the anti-IL-23p19 antibody treatment for one or more gene transcript biomarker(s) of one or more genes selected from GUCA2A, OTOP2, AQP8, SLC26A2, and ADH1C; and
(c)(ii) determining that the patient having or suspected of having ulcerative colitis is healing in response to the anti-IL-23p19 antibody treatment if the expression level in the one or more gene transcript biomarker(s) is increased after the patient receives the anti-IL-23p19 antibody treatment as compared to the expression level of the one or more gene transcript biomarker(s) in the patient who did not receive anti-IL-23p19 antibody treatment.

In a still further aspect of the present invention, there is provided a method for diagnosing stool frequency in a patient in a patient having or suspected of having ulcerative colitis, the method comprising:
determining an expression level of one or more gene transcript biomarker(s) of one or more genes selected from Table 8;
comparing the determined expression level of the one or more gene transcript biomarker(s) to a reference expression level of one or more gene transcript biomarker(s) of one or more genes selected from the above table; and
providing a diagnosis of stool frequency if the biomarker expression level in the patient is changed as compared to the reference expression level.

In a still further aspect of the present invention, there is provided a method for diagnosing stool frequency in a patient in a patient having or suspected of having ulcerative colitis, wherein the method comprises:
determining an expression level of one or more gene transcript biomarker(s) of one or more genes selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1 and Creatine kinase B;
comparing the determined expression level of the one or more gene transcript biomarker(s) to a reference expression level of one or more gene transcript biomarker(s) of one or more genes selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1 and Creatine kinase B; and
providing a diagnosis of stool frequency if the biomarker expression level in the patient is changed as compared to the reference expression level.

In a still further aspect of the present invention, there is provided a method of treating bowel urgency in a patient having or suspected of having ulcerative colitis, the method comprising:
obtaining a first sample from the patient;
analyzing the first sample to detect one or more gene transcript biomarker(s) of one or more genes selected from Table 9;
administering an anti-IL-23p19 antibody to the patient;
obtaining a second sample from the patient;
analyzing the second sample to detect one or more gene transcript biomarker(s) of one or more genes from the above table;
wherein a change in expression level of the one or more gene transcript biomarker(s) detected in the second sample from the expression level of the one or more gene transcript biomarker(s) detected in the first sample indicates a response to the anti-IL-23p19 antibody.

In a still further aspect of the present invention, there is provided a method of treating bowel urgency in a patient having or suspected of having ulcerative colitis, the method comprising:
obtaining a first sample from the patient;
analyzing the first sample to detect one or more gene transcript biomarker(s) of one or more genes selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, and Calpain 13;
administering an anti-IL-23p19 antibody to the patient;
obtaining a second sample from the patient;
analyzing the second sample to detect one or more gene transcript biomarker(s) of one or more genes from one or more gene transcript biomarker(s) of one or more genes selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, and Calpain 13;
wherein a change in expression level of the one or more gene transcript biomarker(s) detected in the second sample from the expression level of the one or more gene transcript biomarker(s) detected in the first sample indicates a response to the anti-IL-23p19 antibody.

In a still further aspect of the present invention, there is provided a gene transcript biomarker panel comprising one or more gene transcript biomarkers of one or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16.

In a still further aspect of the present invention, there is provided a gene transcript biomarker panel according to claim 260, wherein the panel comprises one or more gene transcript biomarkers of one or more genes selected from GUCA2A, OTOP2, AQP8, SLC26A2, and ADH1C.

In a still further aspect of the present invention, there is provided a gene transcript biomarker panel comprising one or more gene transcript biomarker(s) of one or more genes selected from Table 8.

In a still further aspect of the present invention, there is provided a gene transcript biomarker panel comprising one or more gene transcript biomarker(s) of one or more genes selected from S100 calcium binding protein 8, S100 calcium binding protein A12, Cadherin related family member 1, 5100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, and Creatine kinase B.

In a still further aspect of the present invention, there is provided a gene transcript biomarker panel comprising one or more gene transcript biomarker(s) of one or more genes selected from Table 9.

In a still further aspect of the present invention, there is provided a gene transcript biomarker panel comprising one or more gene transcript biomarker(s) of one or more genes selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, and Calpain 13.

In a still further aspect of the present invention, there is provided a method of treating ulcerative colitis in a patient having or suspected of having ulcerative colitis. The method includes: obtaining a first sample from the patient; analyzing the first sample to detect at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; administering an anti-IL-23p19 antibody to the patient; obtaining a second sample from the patient; and analyzing the second sample to detect at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16, wherein a change in expression level of the at least one biomarker detected in the second sample from the expression level of the at least one biomarker detected in the first sample indicates a response to the anti-IL-23p19 antibody.

In another aspect, the present disclosure is directed to a method of identifying a patient having or suspected of having anti-Tumor Necrosis Factor (anti-TNF) therapy resistance (anti-TNF^{R}) as a candidate patient for receiving an anti-IL-23p19 antibody treatment for ulcerative colitis. The method includes: obtaining a sample from the patient; analyzing the sample for at least one biomarker of anti-Tumor Necrosis Factor (anti-TNF) therapy resistance (anti-TNF^{R}); and identifying the patient as a candidate patient for receiving the anti-IL-23p19 antibody treatment based on the analysis of the biomarker.

In another aspect, the present disclosure is directed to a method of treating a patient having or suspected of having ulcerative colitis and who has or is suspected of having anti-Tumor Necrosis Factor (anti-TNF) therapy resistance (anti-TNF^{R}) with an anti-IL-23p19 antibody. The method includes: determining if the patient is anti-TNF^{R}; and treating the patient with the anti-IL-23p19 antibody if the patient is anti-TNF^{R}.

In another aspect, the present disclosure is directed to a method of treating a symptom associated with ulcerative colitis in a patient having or suspected of having ulcerative colitis. The method includes: obtaining a first sample from the patient; analyzing the sample for at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; administering an anti-IL-23p19 antibody to the patient; obtaining second sample from the patient; and analyzing the second sample for at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16.

In another aspect, the present disclosure is directed to a method for diagnosing ulcerative colitis in a patient in a patient having or suspected of having ulcerative colitis. The method includes: (a) determining an expression level of at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16 in a sample obtained from the patient, (b) comparing the determined expression level of the at least one biomarker to a reference expression level of at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; and (c) providing a diagnosis of ulcerative colitis if the biomarker expression level in the patient is increased as compared to the reference expression level or if the biomarker expression level in the patient is decreased as compared to the reference expression level.

In another aspect, the present disclosure is directed to a method of diagnosing ulcerative colitis in a patient having or suspected of having ulcerative colitis. The method includes: (a) using an analyzer unit to determine an expression level of at least one of a biomarker including CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16 in a sample obtained from a patient; (b) using a computing device to compare the determined expression level(s) of the at least one biomarker to a reference expression level of at least one of a biomarker including CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; and (c) providing a diagnosis of ulcerative colitis if the biomarker expression level is increased as compared to the reference expression level or if the biomarker expression level is decreased as compared to the reference expression level.

In another aspect, the present disclosure is directed to a method of determining whether a patient having or suspected of having ulcerative colitis is healing in response to an anti-IL-23p19 antibody treatment. The method includes analyzing a sample obtained from a patient before the patient receives an anti-IL-23p19 antibody treatment for at least one biomarker including CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; analyzing a sample obtained from a patient after the patient receives an anti-IL-23p19 antibody treatment for at least one biomarker including CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; and determining that the patient having or suspected of having ulcerative colitis is healing in response to the anti-IL-23p19 antibody treatment if a change in expression level in the at least one biomarker after the patient receives the anti-IL-23p19 antibody treatment is detected.

In another aspect, the present disclosure is directed to a biomarker panel. The biomarker panel includes at least one biomarker including CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16.

In another aspect, the present disclosure is directed to a method of treating stool frequency in a patient having or suspected of having ulcerative colitis, the method comprising: obtaining a first sample from the patient; analyzing the first sample to detect a biomarker selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, Creatine kinase B, and combinations thereof; administering an anti-IL-23p19 antibody to the patient; obtaining a second sample from the patient; and analyzing the second sample to detect a biomarker selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TTMP metallopeptidase inhibitor 1, Transcobalamin 1, Creatine kinase B, and combinations thereof, wherein a change in expression level of the at least one biomarker detected in the second sample from the expression level of the at least one biomarker detected in the first sample indicates a response to the anti-IL-23p19 antibody.

In another aspect, the present disclosure is directed to a method for diagnosing stool frequency in a patient in a patient having or suspected of having ulcerative colitis, the method comprising: (a) determining an expression level of a biomarker selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TTMP metallopeptidase inhibitor 1, Transcobalamin 1, Creatine kinase B, and combinations thereof in a sample obtained from the patient, (b) comparing the determined expression level of the biomarker to a reference expression level of a biomarker selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, Creatine kinase B, and combinations thereof, and (c) providing a diagnosis of stool frequency if the biomarker expression level in the patient is changed as compared to the reference expression level.

In another aspect, the present disclosure is directed to a method of treating bowel urgency in a patient having or suspected of having ulcerative colitis, the method comprising: obtaining a first sample from the patient; analyzing the first sample to detect a biomarker selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, Calpain 13, and combinations thereof, administering an anti-IL-23p19 antibody to the patient; obtaining a second sample from the patient; and analyzing the second sample to detect a biomarker selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, Calpain 13, and combinations thereof.

In another aspect, the present disclosure is directed to a method of diagnosing bowel urgency in a patient having or suspected of having ulcerative colitis, the method comprising: (a) determining an expression level of a biomarker selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, Calpain 13, and combinations thereof in a sample obtained from the patient, (b) comparing the determined expression level of the biomarker to a reference expression level of a biomarker selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, Calpain 13, and combinations thereof; and (c) providing a diagnosis of bowel urgency if the biomarker expression level in the patient is changed as compared to the reference expression level.

In another aspect, the present disclosure is directed to method of diagnosing stool frequency in a patient having or suspected of having ulcerative colitis, the method comprising: (a) using an analyzer unit to determine an expression level of a biomarker selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, Creatine kinase B, and combinations thereof in a sample obtained from the patient; (b) using a computing device to compare the determined expression level(s) of the biomarker to a reference expression level of a biomarker selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, Creatine kinase B, and combinations thereof, and (c) providing a diagnosis of stool frequency if the biomarker expression level is changed as compared to the reference expression level.

In another aspect, the present disclosure is directed to a method of diagnosing bowel urgency in a patient having or suspected of having ulcerative colitis, the method comprising: (a) using an analyzer unit to determine an expression level of a biomarker selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, Calpain 13, and combinations thereof in a sample obtained from the patient; (b) using a computing device to compare the determined expression level(s) of the biomarker to a reference expression level of a biomarker selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, Calpain 13, and combinations thereof; and (c) providing a diagnosis of bowel urgency if the biomarker expression level is changed as compared to the reference expression level.

In another aspect, the present disclosure is directed to a biomarker panel comprising at least one biomarker comprising S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, and Creatine kinase B.

In another aspect, the present disclosure is directed to a biomarker panel comprising at least one biomarker comprising Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, and Calpain 13.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The disclosure will be better understood, and features, aspects and advantages other than those set forth above will become apparent when consideration is given to the following detailed description thereof. Such detailed description makes reference to the following drawings, wherein:

FIGS. 1A-1D depict differentially expressed genes after mirikizumab treatment.

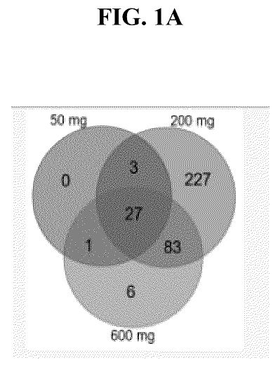

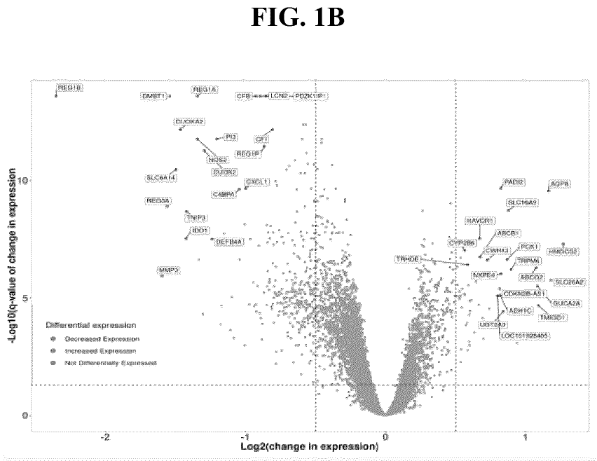

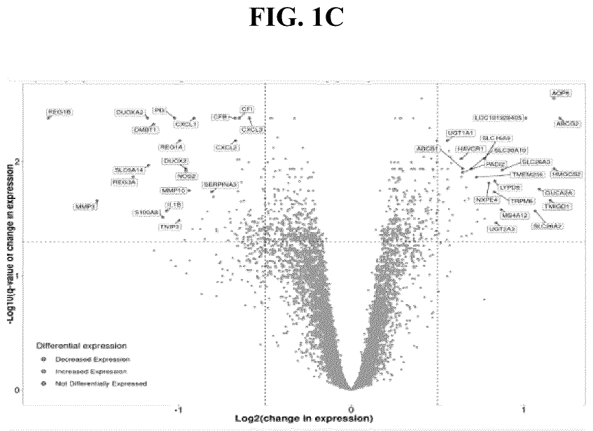

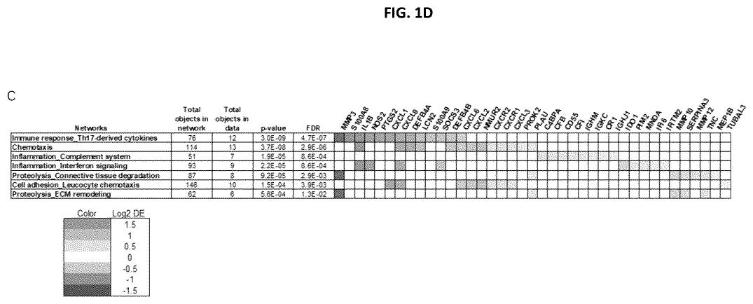

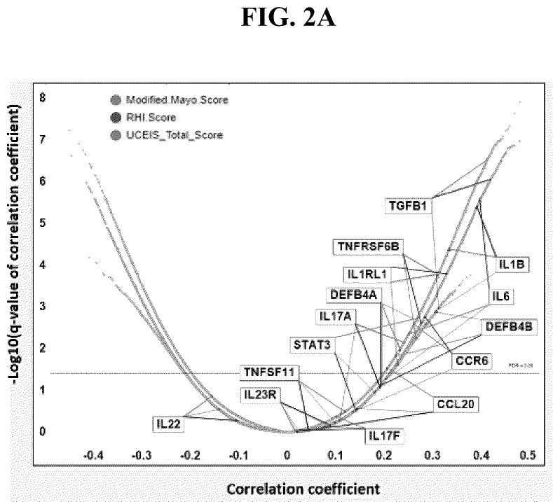

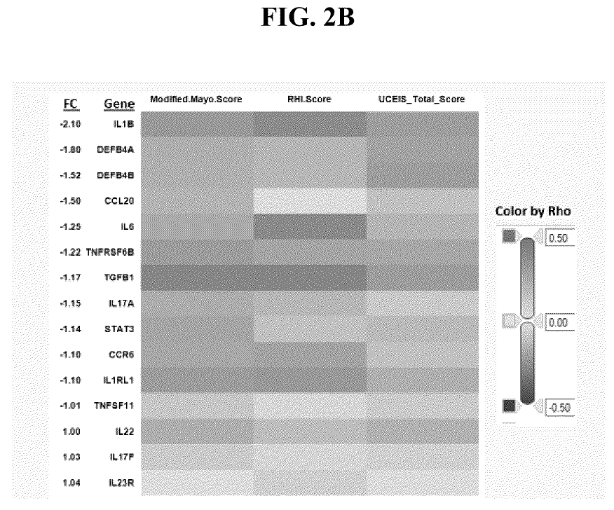

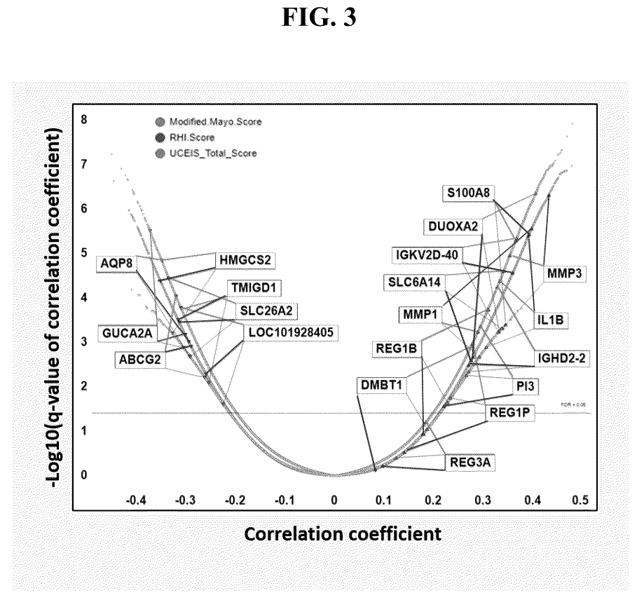

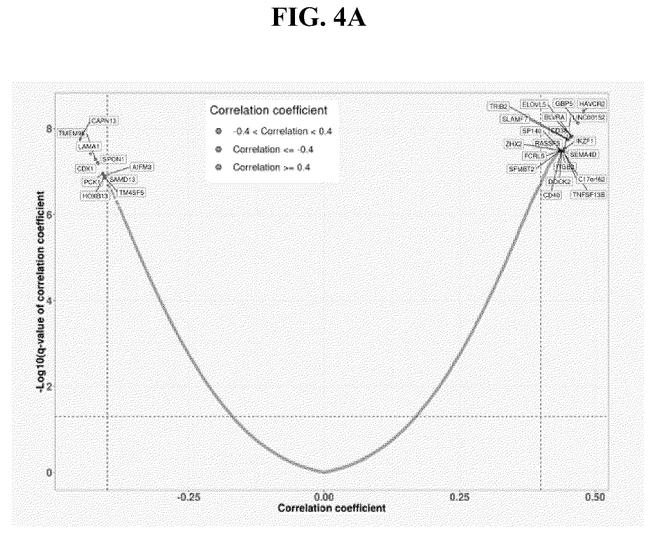

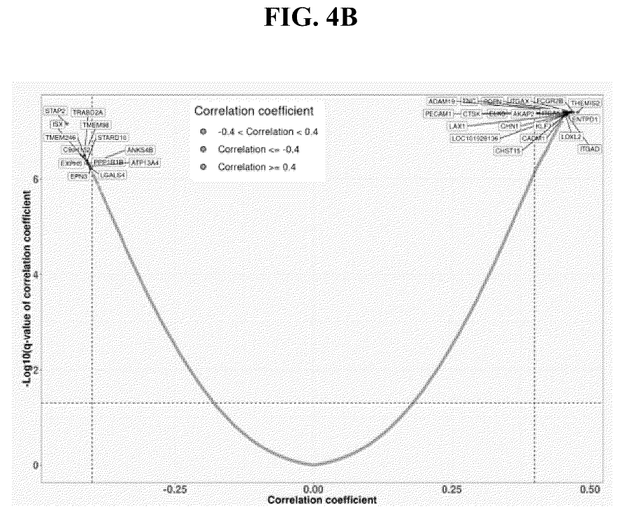

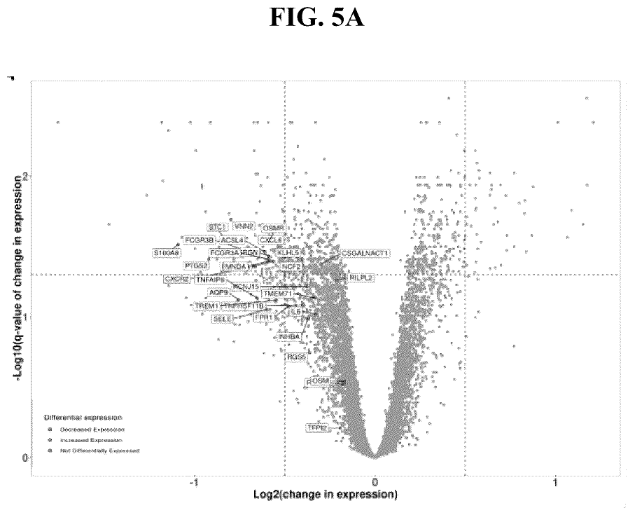

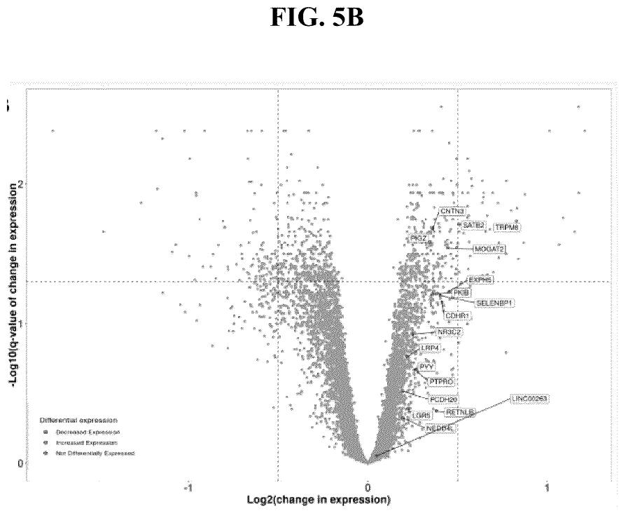

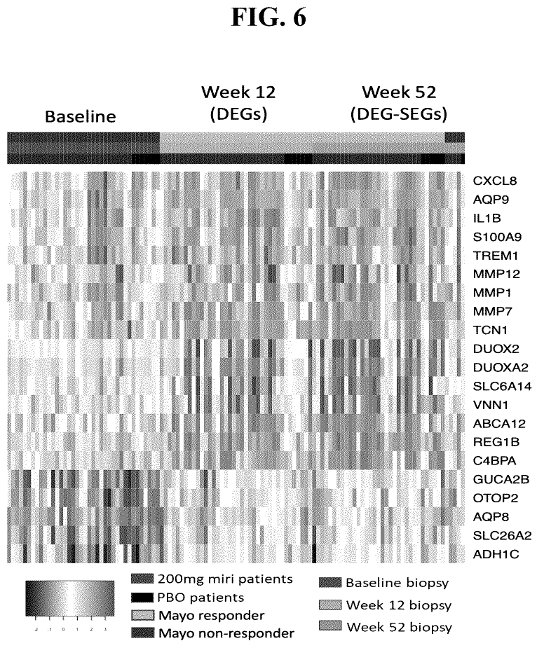

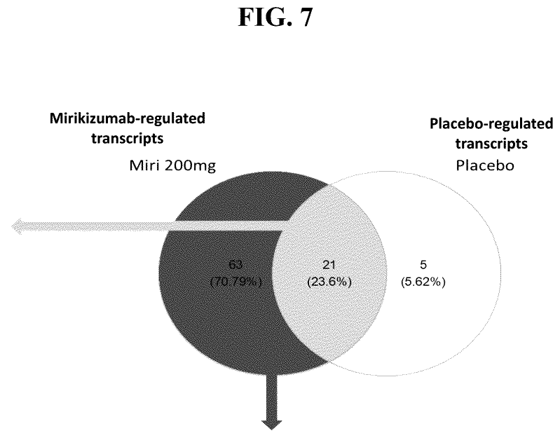

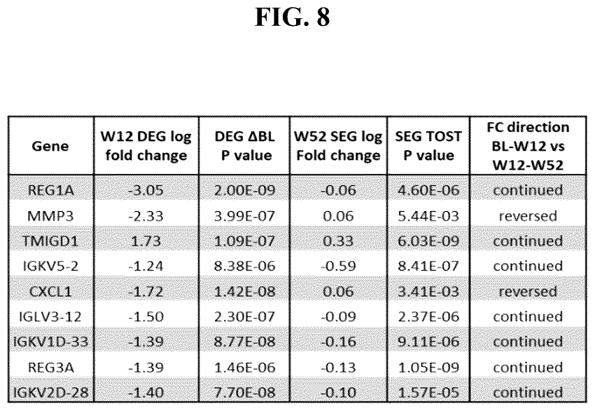

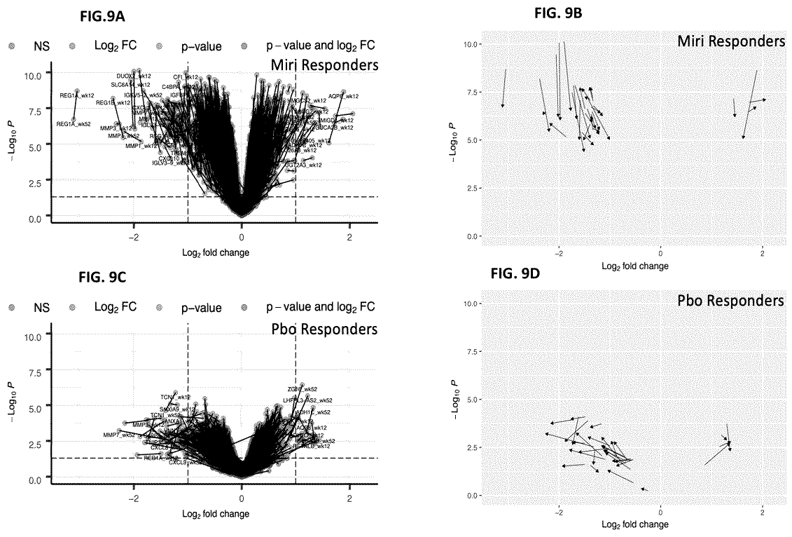

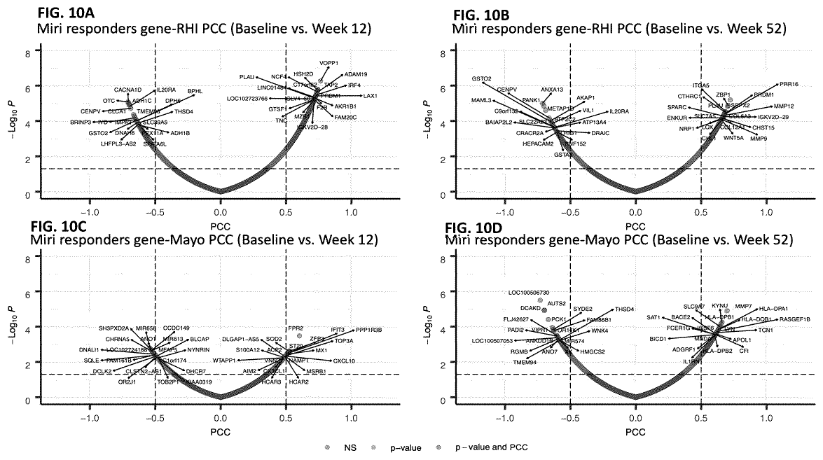

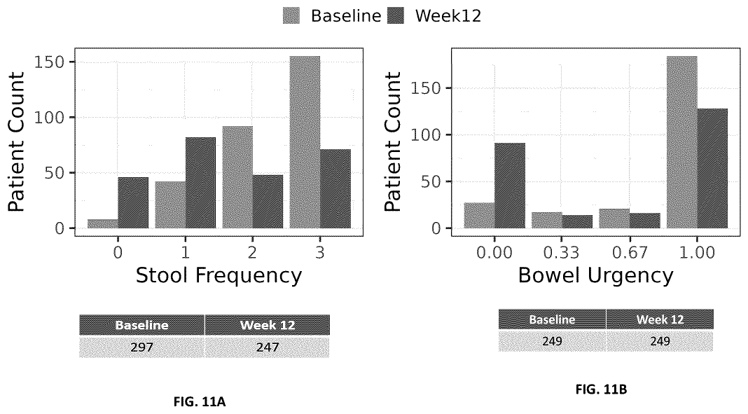

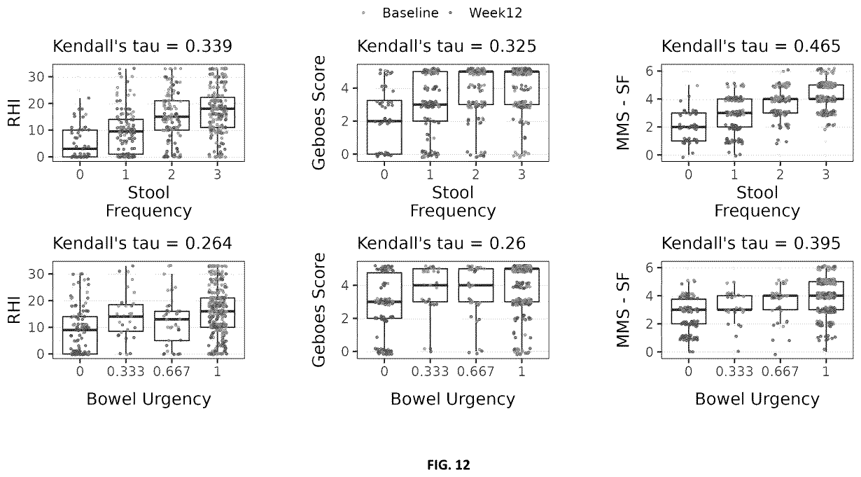

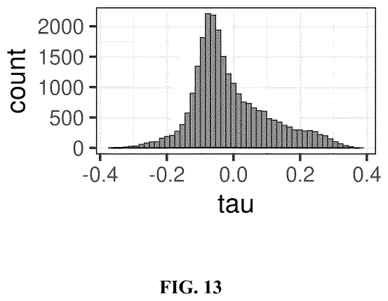

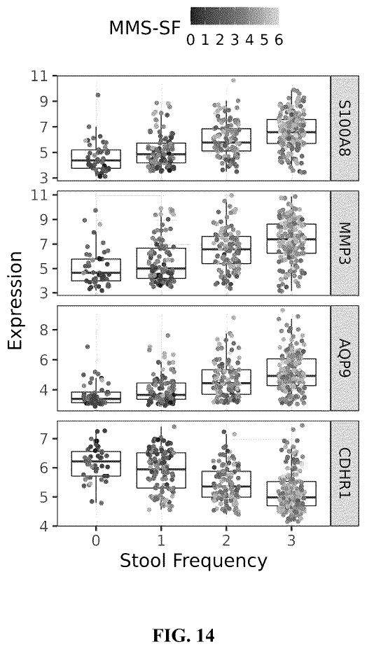

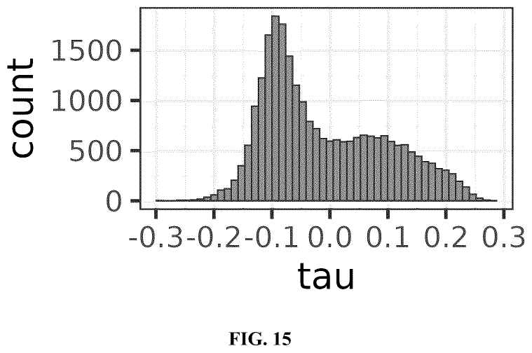

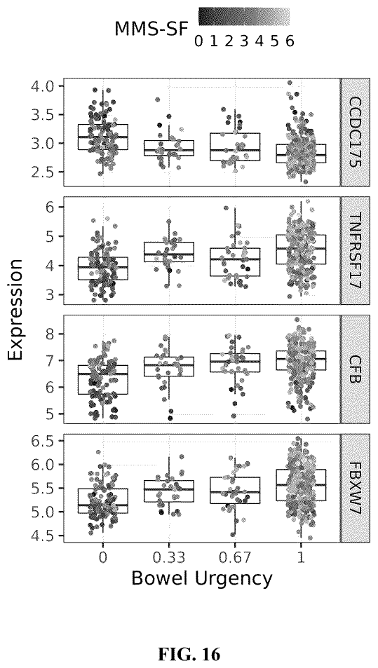

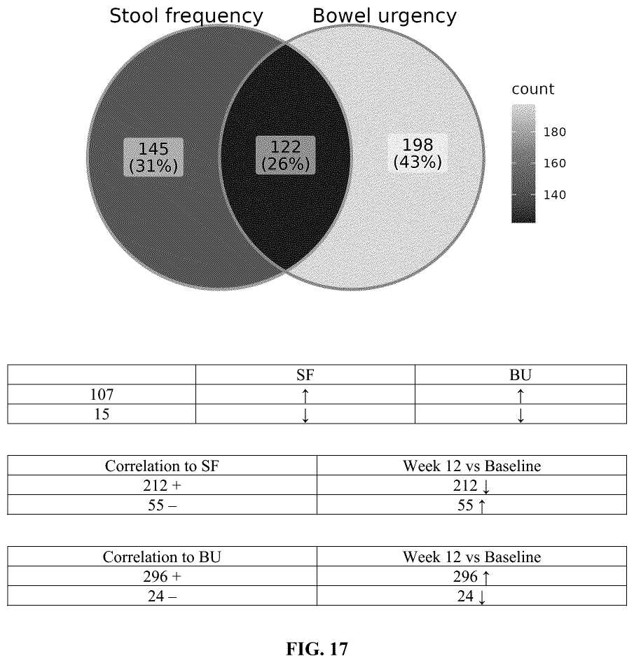

(FIG. 1A) Overlap of genes differentially expressed (log 2 fold change ≥0.5 and FDR ≤0.05) between baseline and Week 12 in mirikizumab treatment groups. No genes showed differential expression based on these fold change and FDR thresholds in the placebo group.

(FIGS. 1B-1C) Differentially expressed genes in the 200 mg mirikizumab group before (FIG. 1B) and after (FIG. 1C) normalizing for placebo are shown as blue circles if decreased and orange circles if increased. The 20 most increased and decreased are labelled.

(FIG. 1D) The most differentially expressed genes in the 200 mg mirikizumab group, after normalizing for placebo, grouped by gene networks as defined by the MetaCore database.

FIGS. 2A and 2B depict correlation between IL-23 pathway transcripts and changes in disease activity scores.

(FIG. 2A) Pearson's correlation coefficients for the differential expression of mirikizumab-regulated genes at Week 12 with change over the same period in modified Mayo score (green), RHI (red), and UCEIS (gray). Genes that are identified by MetaCore database as being upregulated by IL-23 are labelled.

(FIG. 2B) Heatmap of Pearson's correlation coefficients (Rho) for the differential expression of each gene identified above. Fold change values for each gene in the 200 mg mirikizumab dose group after normalization with the change in the placebo group are labeled on the vertical axis.

FIG. 3 depicts correlation between changes in mirikizumab-regulated genes and changes in disease activity scores. Pearson's correlation coefficients for the differential expression of mirikizumab-regulated genes at Week 12 with change over the same period in modified Mayo score (green), RHI (red), and UCEIS (gray). Genes with a differential expression of ≥2-fold in the 200 mg mirikizumab dose group after normalization with change in the placebo group are labelled. All labelled genes that were positively correlated with changes in disease activity scores decreased between baseline and Week 12, and those that were negatively correlated with changes in disease activity scores increased in expression over that period.

FIGS. 4A and 4B depict Mirikizumab-regulated genes most highly correlated with change in disease scores. Pearson's correlation coefficients for the differential expression of each gene between baseline and Week 12 with change over the same period of modified Mayo score

(FIG. 4A) and RHI (FIG. 4B). The 20 most differentially regulated genes are labelled, with pathway analysis summarized in Table 3.

FIGS. 5A and 5B depict Mirikizumab-regulated genes associated with anti-TNFa resistance and response. Change in expression of each gene in the 200 mg mirikizumab dose group between baseline and Week 12 normalized for change in expression in the placebo group over the same period. Differentially expressed genes (log 2 fold change ≥0.5 and FDR≤0.05) are shown with blue circles if decreased and orange circles if increased. Genes associated with resistance (FIG. 5A) and response (FIG. 5B) to anti-TNFa treatment are labelled.

FIG. 6 is a heat map depicting differential gene expression profiles between Week 12 and Week 52 clinical responders to mirikizumab and placebo (“PBO”) responders.

FIG. 7 is a Venn diagram of the differentially expressed genes and the similarly expressed genes present only in mirikizumab responders, present only in PBO responders, and present in both groups.

FIG. 8 depicts the top ten mirikizumab-specific differentially expressed genes and similarly expressed genes (DEG-SEG genes).

FIGS. 9A-9D depict the differential gene expression from Baseline (“BL”) at 12 and 52 Weeks in mirikizumab responders (FIGS. 9A and 9B) and placebo (“PBO”) responders (FIGS. 9C and 9D). The arrows in FIGS. 9B and 9D show the directionality of the change in gene expression.

FIGS. 10A-10D depict the correlation between the cluster of genes with disease activity indices in mirikizumab treated patients. FIG. 10A depicts the cluster of genes in mirikizumab (“Miri”) responders with RHI PCC at week 12. FIG. 10B depicts the cluster of genes in mirikizumab (“Miri”) responders with RHI PCC at week 52. FIG. 10C depicts the cluster of genes in mirikizumab (“Miri”) responders with Mayo PCC indices at week 12. FIG. 10D depicts the cluster of genes in mirikizumab (“Miri”) responders with Mayo PCC indices at week 52. PCC=Pearson's Correlation Coefficient; gray NS=not significant; blue p-value; red p-value and PCC.

FIGS. 11A and 11B are graphs depicting patient reported Stool Frequency (SF) (FIG. 11A) and Bowel Urgency (BU) (FIG. 11B) at baseline and Week 12 of mirikizumab induction.

FIG. 12 are graphs depicting Kendall's tau versus clinical metrics including Robarts Histopathology Index (RHI), Geboes Score, and Modified Mayo Score (MMS) for Stool Frequency and Bowel Urgency.

FIG. 13 is a graph depicting tau distribution for the top 20 genes associated with SF.

FIG. 14 is a graph depicting expression of selected biomarkers (S100A8, MMP3, AQP9, and CDHR1) in relation to MMS-SF scores (Modified Mayo Score—Stool Frequency).

FIG. 15 is a graph depicting tau distribution for the top 20 genes associated with SF.

FIG. 16 is a graph depicting expression of selected biomarkers (CCDCl75, TNFRSF17, CFB, and FBXW7 in relation to Bowel Urgency scores. MMS-SF scores=Modified Mayo Score—Stool Frequency.

FIG. 17 is a Venn diagraph depicting genes associated with Stool Frequency alone (145), Bowel Urgency alone (198), and genes associated with both Stool Frequency and Bowel Urgency (122).

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

UC is a form of colitis, an inflammatory disease of the intestine, usually the colon, which includes characteristic ulcers. Symptoms of active disease usually include diarrhea mixed with blood, usually accompanied with varying degrees of abdominal pain, from mild discomfort to severely painful cramps.

There are a number of methods for assessing the severity of disease, including the Mayo Score, the Modified Mayo Score (MMS) and Ulcerative Colitis Disease Activity Index (UCDAI).

The Mayo score is a composite instrument comprised of the following 4 subscores:
(i) Stool Frequency (SF): The SF subscore is a patient-reported measure. This item reports the number of stools in a 24-hour period, relative to the normal number of stools for that patient in the same period, on a 4-point scale. A stool is defined as a trip to the toilet when the patient has either a bowel movement, or passes blood alone, blood and mucus, or mucus only. The total number of stools passed in a 24-hour period is recorded by the patient. The reference “normal” SF for that patient is typically recorded at the outset of a study or period of observation. Normal SF for that patient is on the reported SF when the patient was in remission or, if the patient has never achieved remission, the reported SF before initial onset of signs and symptoms of UC.

| Stool Frequency Subscore | Score |
|---|---|
| Normal number of stools per day for subject | 0 |
| 1 to 2 stools per day more than normal | 1 |
| 3 to 4 stools per day more than normal | 2 |
| 5 or more stools per day more than normal | 3 |

(ii) Rectal Bleeding: The RB subscore is a patient-reported measure. This item reports the most severe amount of blood passed per rectum for a given day, on a 4-point scale.

| Rectal Bleeding Subscore | Score |
|---|---|
| No blood seen | 0 |
| Streaks of blood with stool | 1 |
| less than half of the time | |
| Obvious blood (more than just streaks) or | 2 |
| streaks of blood with stool most of the time | |
| Blood alone passed | 3 |

(iii) Endoscopic Subscore (ES): The ES is a physician-reported measure that reports the worst appearance of the mucosa on flexible sigmoidoscopy or colonoscopy, on a 4-point scale. Consistent with current clinical practice, friability is excluded from the definition of an ES of 1.

| Endoscopic Subscore | Score |
|---|---|
| Normal or inactive disease | 0 |
| Mild disease (erythema, | 1 |
| decreased vascular pattern) | |
| Moderate disease (marked erythema, | 2 |
| absent vascular pattern, friability, erosions) | |
| Severe disease (spontaneous bleeding, ulceration) | 3 |

(iv) Physician's Global Assessment (PGA): The PGA is a physician-reported measure that summarizes the assessment of the patient's UC disease activity on a 4-point scale.

| Physician's Global Assessment | Score |
|---|---|
| Normal | 0 |
| Mild disease | 1 |
| Moderate disease | 2 |
| Severe disease | 3 |

Each subscore is scored on a 4-point scale, ranging from 0 to 3, to give a maximum Mayo score of 12.

The MMS is a modification made to the original Mayo Index reference (Schroeder et al., New Eng J Med, 317(26):1625-1629, 1987) and includes 3 of the 4 subscores of the Mayo Score. It does not include the Physician's Global Assessment. The MMS evaluates three subscores, each on a scale of 0 to 3 with a maximum total score of 9. Patients who have a Mayo Score of 6-12 or a MMS of 4-9, each with an ES of ≥2, are defined as having moderate to severely active ulcerative colitis.

As used herein, “a subject in need thereof” refers to a subject having, suspected of having, susceptible to and at risk of a specified disease, disorder, or condition. More particularly, in the present disclosure the methods of treating ulcerative colitis and the methods of screening biomarkers is to be used with a subset of subjects who have, are suspected of having, are susceptible to and are at elevated risk for experiencing ulcerative colitis. Such subjects may include, but are not limited to, subjects having, suspected of having, susceptible to and at risk of ulcerative colitis. Subjects having, suspected of having, susceptible to and at risk of ulcerative colitis due to family history, age, environment, and/or lifestyle. In other embodiments, subjects having, suspected of having, susceptible to and at risk of having ulcerative colitis include subjects who have or are suspected of having resistance to anti-TNF treatment for ulcerative colitis. Subjects may have or are suspected of having resistance to anti-TNF treatment for ulcerative colitis due to family history, age, environment, and/or lifestyle.

Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects in need of treatment described herein.

As used herein, “susceptible” and “at risk” refer to having little resistance to a certain disease, disorder or condition, including being genetically predisposed, having a family history of, and/or having symptoms of the disease, disorder or condition.

As used herein, the term “biomarker” refers to any molecule or group of molecules found in a biological sample that can be used to characterize the biological sample or a subject from which the biological sample is obtained. For example, a biomarker may be a molecule or group of molecules whose presence, absence, or relative abundance is: characteristic of a particular cell or tissue type or state; and/or characteristic of a particular pathological condition or state; and/or indicative of the severity of a pathological condition, the likelihood of progression or regression of the pathological condition, and/or the likelihood that the pathological condition will respond to a particular treatment. As another example, the biomarker may be a cell type or a microorganism (such as a bacterium, mycobacterium, fungus, virus, and the like), or a substituent molecule or group of molecules thereof. Biomarkers provided herein can be diagnostic biomarkers that can be used to detect and/or confirm the presence of ulcerative colitis. Biomarkers provided herein can also be monitoring biomarkers that can be serially analyzed to assess the status of ulcerative colitis. Biomarkers provided herein can also be pharmacodynamic biomarkers that can be used to determine a patient's response to an anti-IL-23p19 antibody treatment. Biomarkers provided herein can also be predictive biomarkers that can be used to predict or identify an individual or group of individuals more likely to experience a favorable or unfavorable effect from an anti-IL-23p19 antibody treatment. Biomarkers provided herein can also be safety biomarkers that are measured before and/or after anti-IL-23p19 antibody administration to indicate the likelihood, presence, or extent of a toxicity to anti-IL-23p19 antibody. Biomarkers provided herein can also be prognostic biomarkers to identify ulcerative colitis progression and/or recurrence. Biomarkers provided herein can also be susceptibility/risk biomarkers that can indicates the potential for an individual to develop ulcerative colitis but who has not been diagnosed as having ulcerative colitis. Biomarkers provided herein can also be surrogate biomarkers that explain the clinical outcome following anti-IL-23p19 antibody treatment.

As used herein, the term “gene transcript biomarker” refers to the gene expression products that correspond with a particular gene, for example, a RNA transcript expressed by a particular gene. Gene transcript biomarkers may be used as described above in respect of biomarkers more generally. Gene transcript biomarkers provided herein can be diagnostic biomarkers that can be used to detect and/or confirm the presence of ulcerative colitis. Gene transcript biomarkers provided herein can also be monitoring biomarkers that can be serially analyzed to assess the status of ulcerative colitis. Gene transcript biomarkers provided herein can also be pharmacodynamic biomarkers that can be used to determine a patient's response to an anti-IL-23p19 antibody treatment. Gene transcript biomarkers provided herein can also be predictive biomarkers that can be used to predict or identify an individual or group of individuals more likely to experience a favorable or unfavorable effect from an anti-IL-23p19 antibody treatment. Gene transcript biomarkers provided herein can also be safety biomarkers that are measured before and/or after anti-IL-23p19 antibody administration to indicate the likelihood, presence, or extent of a toxicity to anti-IL-23p19 antibody. Gene transcript biomarkers provided herein can also be prognostic biomarkers to identify ulcerative colitis progression and/or recurrence. Gene transcript biomarkers provided herein can also be susceptibility/risk biomarkers that can indicates the potential for an individual to develop ulcerative colitis but who has not been diagnosed as having ulcerative colitis. Gene transcript biomarkers provided herein can also be surrogate biomarkers that explain the clinical outcome following anti-IL-23p19 antibody treatment. The terms “biomarker” and “gene transcript biomarker” are used interchangeably herein.

As used herein, “expression level of a biomarker (or gene transcript biomarker)” refers to the process by which a gene product is synthesized from a gene encoding the biomarker as known by those skilled in the art. The gene product can be, for example, RNA (ribonucleic acid) and protein. Expression level can be quantitatively measured by methods known by those skilled in the art such as, for example, northern blotting, amplification, polymerase chain reaction, microarray analysis, tag-based technologies (e.g., serial analysis of gene expression and next generation sequencing such as whole transcriptome shotgun sequencing or RNA-Seq), Western blotting, enzyme linked immunosorbent assay (ELISA), and combinations thereof.

As used herein, “a reference expression level” of a biomarker or gene transcript refers to the expression level of a biomarker established for a subject without ulcerative colitis, expression level of a biomarker in a normal/healthy subject without ulcerative colitis as determined by a medical professional and/or research professional using established methods as described herein, and/or a known expression level of a biomarker obtained from literature. The reference expression level of the biomarker can also refer to the expression level of the biomarker established for any combination of subjects such as a subject without ulcerative colitis, expression level of the biomarker in a normal/healthy subject without ulcerative colitis, and expression level of the biomarker for a subject without ulcerative colitis at the time the sample is obtained from the subject, but who later exhibits without ulcerative colitis. The reference expression level of the biomarker can also refer to the expression level of the biomarker obtained from the subject to which the method is applied. As such, the change within a subject from visit to visit can indicate an increased or decreased risk for ulcerative colitis. For example, a plurality of expression levels of a biomarker can be obtained from a plurality of samples obtained from the same subject and used to identify differences between the pluralities of expression levels in each sample. Thus, in some embodiments, two or more samples obtained from the same subject can provide an expression level(s) of a blood biomarker and a reference expression level(s) of the blood biomarker. The reference expression level can also refer to the expression level of a biomarker in a “placebo responder”. As used herein a “placebo responder” is a subject having ulcerative colitis as determined by a medical professional and/or research professional using established methods as described herein who demonstrates clinical improvement, but who is not administered an anti-IL-23p19 antibody. Without being bound by theory, it is believed that placebo responders demonstrate improvement due to lifestyle changes made by the placebo responder in response to an ulcerative colitis diagnosis and/or counseling and/or medical follow-up.

Examples of anti-IL-23p19 antibodies that may be used in the methods, treatments and uses of the present invention include guselkumab, tildrakizumab, risankizumab, mirikizumab and brazikumab.

Guselkumab, CAS Registry No. 1350289-85-8, is a fully human IgG₁ lambda monoclonal antibody that binds to the p19 subunit of human IL-23. The antibody and methods of making same are described in U.S. Pat. No. 7,935,344.

Tildrakizumab, CAS Registry No. 1326244-10-3, is a humanized, IgG₁ kappa monoclonal antibody targeting the p19 subunit of human IL-23. The antibody and methods of making same are described in U.S. Pat. No. 8,293,883.

Risankizumab, CAS Registry No. 1612838-76-2, is a humanized, IgG₁ kappa monoclonal antibody targeting the p19 subunit of human IL-23. The antibody and methods of making same are described in U.S. Pat. No. 8,778,346.

Mirikizumab (also referred to herein as “miri”), CAS Registry No. 1884201-71-1, is a humanized, IgG₄-kappa monoclonal antibody targeting the p19 subunit of human IL-23. The antibody and methods of making same are described in U.S. Pat. No. 9,023,358. Mirikizumab is particularly suitable for use in many aspects of the present invention.

Suitable anti-IL-23p19 antibody induction dosage includes from about 50 mg to about 600 mg. A particularly suitable dosage is a 300 mg induction dose of an anti-IL-23p19 antibody. The induction dose of an anti-IL-23p19 antibody is suitably administered intravenously. Suitably, a patient is administered 50 mg to 600 mg, preferably 300 mg of an induction dose every 4 weeks for 12 weeks. The induction dose(s) may be followed by at least one maintenance dose ranging from about 150 mg to about 400 mg, preferably 200 mg, of an anti-IL-23p19 antibody. A particularly suitable dosage is a 200 mg maintenance dose of an anti-IL-23p19 antibody. Suitably, a patient is administered 150 mg to 400 mg of a maintenance dose every 4 weeks or every 12 weeks. Administration of at least one induction dose of an anti-IL-23p19 antibody to a patient in need thereof in an induction period is intended to induce a desired therapeutic effect, the desired therapeutic effect being clinical remission, clinical response, endoscopic remission, endoscopic healing and/or symptomatic remission. If the patient achieves a desired therapeutic effect at the end of the induction period, he/she is subsequently administered at least one maintenance dose to maintain at least one of the therapeutic effect(s) obtained during the induction period, the therapeutic effect(s) being clinical remission, clinical response, endoscopic remission, endoscopic healing and/or symptomatic remission. There is no minimum or maximum duration of the induction period but it is typically 4, 8 or 12 weeks in duration, with the end of induction period being an end-of-induction assessment typically occurring 4 or 8 weeks after the last induction dose has been administered. Administration of the induction dose can be extended termed “extended induction dose” to distinguish it from the initial induction dose—if the patient does not achieve clinical response at the end of the initial induction period. If the patient achieves clinical response at the end of the extended induction period, at least one maintenance dose of the anti-IL-23p19 antibody is administered to maintain clinical response or other desired therapeutic effect(s) such as clinical remission, endoscopic remission, endoscopic healing and/or symptomatic remission. The first maintenance dose is administered 4-12 weeks after the last extended induction dose is administered to the patient. The 4-12 week period accommodates variation in the period between the administration of last extended induction dose and the end of extended-induction assessment. The maintenance dose(s) are administered at 4, 8 or 12 week interval(s) after administration of the first maintenance dose. Maintenance dose(s) can be administered by subcutaneous injection. If the patient develops a loss of response during the maintenance period, one, two or three rescue dose(s) of the anti-IL-23p19 antibody are administered to the patient, wherein one or more further maintenance dose(s) of the anti-IL-23p19 antibody are administered to the patient if the patient achieves clinical response 4-12 weeks after the last rescue dose is administered, wherein loss of response is defined as: (a) >2-point increase from baseline in the combined stool frequency (SF) and rectal bleeding (RB) scores (b) combined SF and RB score of >4, on 2 consecutive visits >7 days apart with confirmation of negative Clostridium difficile testing and (c) endoscopic subscore (ES) of 2 or 3, and wherein clinical response is defined as achieving a decrease in the 9 point Modified Mayo Score (MMS) subscore of >2 points and >30-35% from baseline, with either a decrease of rectal bleeding (RB) subscore of >1 or a RB subscore of 0 or 1.

The methods disclosed herein can further include obtaining three or more samples from the patient. It is particularly suitable to obtain multiple samples from a patient, a reference subject, and a placebo responder for analysis of samples to determine whether expression levels of biomarkers change, remain changed over time, are maintained over time, and the like.

Suitable samples include whole blood, plasma, serum, tissue biopsy, fecal samples, and combinations thereof. Particularly suitable tissue biopsy samples include a colonic tissue biopsy sample or a rectal tissue biopsy sample. The colonic tissue biopsy is from a tissue selected from the group consisting of the terminal ileum, the ascending colon, the descending colon, and the sigmoid colon. The colonic tissue biopsy may be from a non-inflamed colonic area or from an inflamed colonic area. The biopsy may be obtained from the edge of ulcers, obtained from the edge of erosions, obtained spaced throughout affected mucosa, and combinations thereof.

In respect of embodiments wherein a patient is administered an anti-IL23p19 antibody, the first sample is taken before or simultaneous with administration of the anti-IL-23p19 antibody and the second sample is taken at least two weeks, at least four weeks, at least eight weeks, at least twelve weeks, at least sixteen weeks, at least twenty weeks, at least twenty-four weeks, at least twenty-eight weeks, at least thirty weeks, at least thirty-two weeks, at least thirty-six weeks, at least forty weeks, at least forty-four weeks, at least forty-eight weeks, or at least fifty-two weeks, after the first administration of the anti-IL-23p19 antibody. Alternatively, samples may be obtained at about 4 weeks following anti-IL-23p19 antibody administration, at about 12 weeks following anti-IL-23p19 antibody administration, at about 52 weeks following anti-IL-23p19 antibody administration, and combinations thereof. Samples can further be obtained after 52 weeks following anti-IL-23p19 antibody administration. Samples can further be obtained at other intervals including daily, weekly, monthly, and yearly.

Expression (and expression level) can be determined by microarray analysis. Other suitable methods for determining expression include amplification (polymerase chain reaction), northern blot, southern blot, in situ hybridization, immunoassays including western blot, enzyme-linked immunosorbent assay (ELISA), enzyme-linked fluorescence assay (ELFA), immunoprecipitation, immunohistochemistry, and combinations thereof.

The methods disclosed herein can further include analyzing a tissue sample using histopathology. Tissue samples can be processed and stained for bright field microscopy using H & E stain, Romanowsky staining, and even unstained tissue samples. Tissue samples can also be stained using an antibody that specifically binds to a biomarker to be detected. The antibody can include a label such as a fluorescent label and the tissue can be examined by exposing the tissue sample to ultraviolet light. The biomarker antibody can be directly labeled with a fluorescent label or detected using a fluorescently labeled second antibody that specifically binds the biomarker antibody. Tissue samples can be labeled to detect a single biomarker or multiple biomarkers. Tissue samples can also be analyzed using spatial transcriptomics to determine subcellular localization of the biomarker mRNAs.

In one aspect, the present disclosure is directed to a method of treating ulcerative colitis in a patient having or suspected of having ulcerative colitis. The method includes: obtaining a first sample from the patient; analyzing the first sample to detect at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; administering an anti-IL-23p19 antibody to the patient; obtaining a second sample from the patient; and analyzing the second sample to detect at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16, wherein a change in expression level of the at least one biomarker detected in the second sample from the expression level of the at least one biomarker detected in the first sample indicates a response to the anti-IL-23p19 antibody. Particularly suitable biomarkers include at least one of CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, and combinations thereof. In one embodiment, the biomarker is increased following the anti-IL-23p19 antibody treatment and includes one of GUCA2B, OTOP2, AQP8, SLC26A2, ADH1C, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, ZG16, and combinations thereof. In one embodiment, the biomarker is decreased following the anti-IL-23p19 antibody treatment and includes one of CXCL8, AQP9, IL1B, S100A9, TREM1, MMP12, MMP1, MMP7, TCN1, DUOX2, DUOXA2, SLC6A14, VNN1, ABCA12, REG1B, C4BPA, REG3A, DMBT1, REG1P, S100A8, IGKV2D-40, PI3, TNIP3, REG1A, IDO1, NOS2, MMP10, CXCL1, PTGS2, and combinations thereof.

In one embodiment, a change in the expression detected in the second sample from the expression detected in the first sample indicates that the anti-IL-23p19 antibody administration should be continued. A change in the expression can be an increase in the expression in the second (or subsequent) sample as compared to the expression in the first sample. A change in the expression can also be a decrease in the second (or subsequent) sample as compared to the expression in the first sample. The change in expression level in the sample(s) obtained from the patient administered the anti-IL-23p19 antibody can further be compared to one of an expression level in a sample(s) obtained from a healthy subject (a subject who is not suspected of having or has ulcerative colitis) and an expression level in a sample(s) obtained from a patient having or suspected of having ulcerative colitis who is not administered an anti-IL-23p19 antibody.

In another embodiment, change in the expression level detected in the second sample from the expression level detected in the first sample indicates that the anti-IL-23p19 antibody administration should be discontinued. A change in the expression level can be an increase in the expression level in the second (or subsequent) sample as compared to the expression level in the first sample. A change in the expression level can also be a decrease in the second (or subsequent) sample as compared to the expression level in the first sample. The change in expression level in the sample(s) obtained from the patient administered the anti-IL-23p19 antibody can further be compared to one of an expression level in a sample(s) obtained from a healthy subject (a subject who is not suspected of having or has ulcerative colitis) and an expression level in a sample(s) obtained from a patient having or suspected of having ulcerative colitis who is not administered an anti-IL-23p19 antibody.

The method can further include analyzing clinical metrics including modified Mayo Score (MMS), Total Mayo Score, Mayo Endoscopic Subscore, Ulcerative Colitis Endoscopic Index of Severity (UCEIS) Total Score, Geboes Score, Robarts Histopathology Index (RHI), and combinations thereof.

In another aspect, the present disclosure is directed to a method of identifying a patient having or suspected of having anti-Tumor Necrosis Factor (anti-TNF) therapy resistance (anti-TNF^{R}) as a candidate patient for receiving anti-IL-23p19 antibody treatment for ulcerative colitis. The method includes: obtaining a sample from the patient; analyzing the sample for at least one biomarker of anti-Tumor Necrosis Factor (anti-TNF) therapy resistance (anti-TNF^{R}); and identifying the patient as a candidate patient for receiving anti-IL-23p19 antibody treatment based on the analysis of the biomarker.

Suitable biomarkers of anti-Tumor Necrosis Factor (anti-TNF) therapy resistance include OSMR, FCGR3, CXCL6, interleukin-11, interleukin-24, interleukin-13RA2, FAP, TWIST1, and WNT2.

The method can further include analyzing a sample obtained from the patient for at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16.

The method can further include administering an anti-IL-23p19 antibody to the patient as described herein.

The method can further include analyzing clinical metrics including modified Mayo Score (MMS), Total Mayo Score, Mayo Endoscopic Subscore, Ulcerative Colitis Endoscopic Index of Severity (UCEIS) Total Score, Geboes Score, Robarts Histopathology Index (RHI), and combinations thereof.

In another aspect, the present disclosure is directed to a method of treating a patient having or suspected of having ulcerative colitis and who has or is suspected of having anti-Tumor Necrosis Factor (anti-TNF) therapy resistance (anti-TNF^{R}) with an anti-IL-23p19 antibody. The method includes: determining if the patient is anti-TNF^{R}; and treating the patient with an anti-IL-23p19 antibody if the patient is anti-TNF^{R}.

The sample obtained from the patient is analyzed for anti-TNF^{R} transcripts including OSMR, FCGR3, CXCL6, interleukin-11, interleukin-24, interleukin-13RA2, FAP, TWIST1, and WNT2.

The method can further include analyzing samples obtained before anti-IL-23p19 antibody administration and following anti-IL-23p19 antibody administration for at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16.

The method can further include analyzing clinical metrics including modified Mayo Score (MMS), Total Mayo Score, Mayo Endoscopic Subscore, Ulcerative Colitis Endoscopic Index of Severity (UCEIS) Total Score, Geboes Score, Robarts Histopathology Index (RHI), and combinations thereof.

In another aspect, the present disclosure is directed to a method of treating a symptom associated with ulcerative colitis in a patient having or suspected of having ulcerative colitis. The method includes: obtaining a first sample from the patient; analyzing the sample for at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; administering an anti-IL-23p19 antibody to the patient; obtaining second sample from the patient; and analyzing the second sample for at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16.

Symptoms of ulcerative colitis include at least one of abdominal pain/discomfort, blood in stool, pus in stool, fever, weight loss, rectal bleeding, frequent diarrhea, recurrent diarrhea, fatigue, reduced appetite, and tenesmus (urgency).

The method can further include analyzing clinical metrics including modified Mayo Score (MMS), Total Mayo Score, Mayo Endoscopic Subscore, Ulcerative Colitis Endoscopic Index of Severity (UCEIS) Total Score, Geboes Score, Robarts Histopathology Index (RHI), and combinations thereof.

In another aspect, the present disclosure is directed to a method for diagnosing ulcerative colitis in a patient having or suspected of having ulcerative colitis. The method includes: (a) determining an expression level of at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16 in a sample obtained from the patient, (b) comparing the determined expression level of the at least one biomarker to a reference expression level of at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; and (c) providing a diagnosis of ulcerative colitis if the biomarker expression level in the patient is increased as compared to the reference expression level or if the biomarker expression level in the patient is decreased as compared to the reference expression level.

The method can further include analyzing clinical metrics including modified Mayo Score (MMS), Total Mayo Score, Mayo Endoscopic Subscore, Ulcerative Colitis Endoscopic Index of Severity (UCEIS) Total Score, Geboes Score, Robarts Histopathology Index (RHI), and combinations thereof.

In another aspect, the present disclosure is directed to a method of diagnosing ulcerative colitis in a patient having or suspected of having ulcerative colitis. The method includes: (a) using an analyzer unit to determine an expression level of at least one of a biomarker including CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16 in a sample obtained from a patient; (b) using a computing device to compare the determined expression level(s) of the at least one biomarker to a reference expression level of at least one of a biomarker including CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; and (c) providing a diagnosis of ulcerative colitis if the biomarker expression level is increased as compared to the reference expression level or if the biomarker expression level is decreased as compared to the reference expression level.

The method can further include using the computing device to establish an aid for diagnosing ulcerative colitis in the subject based on the result of the comparison to the reference biomarker.

The method can further include using the computing device to compare the determined amount(s) of the at least one of a biomarker from the sample to the reference amount(s) of the at least one of the biomarker, wherein the comparison is carried out automatically.

The method can further include using the analyzer unit to measure binding of a ligand to the at least one biomarker of CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16.

The method can further include using the computing device to calculate an amount of the measured binding of the ligand.

The method can further include using the analyzer unit to determine the amount of the at least one biomarker including CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16 based on the calculated amount of the measured binding of the ligand.

In another aspect, the present disclosure is directed to a method of determining whether a patient having or suspected of having ulcerative colitis is healing in response to anti-IL-23p19 antibody treatment. The method includes analyzing a sample obtained from a patient before the patient receives anti-IL-23p19 antibody treatment for at least one biomarker including CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; analyzing a sample obtained from a patient after the patient receives the anti-IL-23p19 antibody treatment for at least one biomarker including CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; and determining that the patient having or suspected of having ulcerative colitis is healing in response to the anti-IL-23p19 antibody treatment if a change in expression level in the at least one biomarker after the patient receives the anti-IL-23p19 antibody treatment is detected.

The method can further include analyzing a sample obtained from a patient having or suspected of having ulcerative colitis who did not receive anti-IL-23p19 antibody treatment for at least one biomarker including CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16.

The method can further include analyzing at least one biomarker including GUCA2A, OTOP2, AQP8, SLC26A2, and ADH1C, wherein an expression level of at least one of GUCA2A, OTOP2, AQP8, SLC26A2, and ADH1C after anti-IL-23p19 antibody treatment is increased as compared to an expression level of at least one of GUCA2A, OTOP2, AQP8, SLC26A2, and ADH1C before anti-IL-23p19 antibody treatment in the patient who is administered anti-IL-23p19 antibody.

The method can further include analyzing an expression level of at least one biomarker including GUCA2A, OTOP2, AQP8, SLC26A2, and ADH1C in a patient who is not administered an anti-IL-23p19 antibody; comparing the expression level of the at least one biomarker in the patient who is not administered anti-IL-23p19 antibody to an expression level of at least one of GUCA2A, OTOP2, AQP8, SLC26A2, and ADH1C in a patient administered anti-IL-23p19 antibody treatment; and determining that the patient administered anti-IL-23p19 antibody treatment is healing if the biomarker expression level in the patient administered anti-IL-23p19 antibody treatment is increased as compared to the expression level of the biomarker in the patient who did not receive anti-IL-23p19 antibody treatment.

In another aspect, the present disclosure is directed to a biomarker panel. The biomarker panel includes at least one biomarker including CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16.

In one aspect, the biomarker panel includes at least one of GUCA2A, OTOP2, AQP8, SLC26A2, and ADH1C.

In another aspect, the present disclosure is directed to a method of treating stool frequency in a patient having or suspected of having ulcerative colitis. The method includes obtaining a first sample from the patient; analyzing the first sample to detect a biomarker selected from Table 8; administering an anti-IL-23p19 antibody to the patient; obtaining a second sample from the patient; and analyzing the second sample to detect a biomarker selected from Table 8, wherein a change in expression level of the at least one biomarker detected in the second sample from the expression level of the at least one biomarker detected in the first sample indicates a response to the anti-IL-23p19 antibody.

Particularly suitable biomarkers selected from Table 8 include S100 calcium binding protein 8, 5100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, and Creatine kinase B.

The method includes administering an anti-IL-23p19 antibody to the patient as described herein.

The method can further include analyzing a tissue sample.

The method can further include analyzing clinical metrics including modified Mayo Score (MMS), Total Mayo Score, Mayo Endoscopic Subscore, Ulcerative Colitis Endoscopic Index of Severity (UCEIS) Total Score, Geboes Score, Robarts Histopathology Index (RHI), and combinations thereof.

In another aspect, the present disclosure is directed to a method for diagnosing stool frequency in a patient in a patient having or suspected of having ulcerative colitis, the method comprising: (a) determining an expression level of a biomarker selected from Table 8 in a sample obtained from the patient, (b) comparing the determined expression level of the biomarker to a reference expression level of a biomarker selected from Table 8; and (c) providing a diagnosis of stool frequency if the biomarker expression level in the patient is changed as compared to the reference expression level.

Particularly suitable biomarkers to select from Table 8 include S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, Creatine kinase B, and combinations thereof.

The method can further include analyzing a tissue sample.

The method can further include analyzing clinical metrics including modified Mayo Score (MMS), Total Mayo Score, Mayo Endoscopic Subscore, Ulcerative Colitis Endoscopic Index of Severity (UCEIS) Total Score, Geboes Score, Robarts Histopathology Index (RHI), and combinations thereof.

In another aspect, the present disclosure is directed to a method of treating bowel urgency in a patient having or suspected of having ulcerative colitis. The method includes: obtaining a first sample from the patient; analyzing the first sample to detect a biomarker selected from Table 9; administering an anti-IL-23p19 antibody to the patient; obtaining a second sample from the patient; and analyzing the second sample to detect a biomarker selected from Table 9, wherein a change in expression level of the biomarker detected in the second sample from the expression level of the biomarker detected in the first sample indicates a response to the anti-IL-23p19 antibody.

Particularly suitable biomarkers to select from Table 9 include Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, Calpain 13, and combinations thereof.

The method includes administering an anti-IL-23p19 antibody to the patient as described herein.

The method can further include analyzing a tissue sample.

The method can further include analyzing clinical metrics including modified Mayo Score (MMS), Total Mayo Score, Mayo Endoscopic Subscore, Ulcerative Colitis Endoscopic Index of Severity (UCEIS) Total Score, Geboes Score, Robarts Histopathology Index (RHI), and combinations thereof.

In another aspect, the present disclosure is directed to a method of diagnosing bowel urgency in a patient having or suspected of having ulcerative colitis. The method includes: (a) determining an expression level of a biomarker selected from Table 9 in a sample obtained from the patient, (b) comparing the determined expression level of the biomarker to a reference expression level of a biomarker selected from Table 9; and (c) providing a diagnosis of bowel urgency if the biomarker expression level in the patient is changed as compared to the reference expression level.

Particularly suitable biomarkers to select from Table 9 include Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, Calpain 13, and combinations thereof.

The method can further include analyzing a tissue sample.

The method can further include analyzing clinical metrics including modified Mayo Score (MMS), Total Mayo Score, Mayo Endoscopic Subscore, Ulcerative Colitis Endoscopic Index of Severity (UCEIS) Total Score, Geboes Score, Robarts Histopathology Index (RHI), and combinations thereof.

In another aspect, the present disclosure is directed to a method of diagnosing stool frequency in a patient having or suspected of having ulcerative colitis. The method includes: (a) using an analyzer unit to determine an expression level of a biomarker selected from Table 8 in a sample obtained from the patient; (b) using a computing device to compare the determined expression level(s) of the biomarker to a reference expression level of a biomarker selected from Table 8; and (c) providing a diagnosis of stool frequency if the biomarker expression level is changed as compared to the reference expression level.

Particularly suitable biomarkers selected from Table 8 include S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, Creatine kinase B, and combinations thereof.

The method can further include analyzing a tissue sample.

The method can further include analyzing clinical metrics including modified Mayo Score (MMS), Total Mayo Score, Mayo Endoscopic Subscore, Ulcerative Colitis Endoscopic Index of Severity (UCEIS) Total Score, Geboes Score, Robarts Histopathology Index (RHI), and combinations thereof.

In another aspect, the present disclosure is directed to a method of diagnosing bowel urgency in a patient having or suspected of having ulcerative colitis. The method includes: (a) using an analyzer unit to determine an expression level of a biomarker selected from Table 9 in a sample obtained from the patient; (b) using a computing device to compare the determined expression level(s) of the biomarker to a reference expression level of a biomarker selected from Table 9; and (c) providing a diagnosis of bowel urgency if the biomarker expression level is changed as compared to the reference expression level.

Particularly suitable biomarkers to select from Table 9 include Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, Calpain 13, and combinations thereof.

The method can further include analyzing a tissue sample.

The method can further include analyzing clinical metrics including modified Mayo Score (MMS), Total Mayo Score, Mayo Endoscopic Subscore, Ulcerative Colitis Endoscopic Index of Severity (UCEIS) Total Score, Geboes Score, Robarts Histopathology Index (RHI), and combinations thereof.

In another aspect, the present disclosure is directed to a biomarker panel including at least one biomarker selected from Table 8. Particularly suitable biomarkers to select for the biomarker panel include S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, and Creatine kinase B.

In another aspect, the present disclosure is directed to a biomarker panel including at least one biomarker selected from Table 9. Particularly suitable biomarkers to select for the biomarker panel include Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, and Calpain 13.

### EXAMPLES

### Materials and Methods

### Study Design and Participants

A multicenter, randomized, double-blind, parallel-arm, placebo-controlled trial was conducted at 75 sites in 14 countries (Australia, Belgium, Canada, Czech Republic, Denmark, Georgia, Hungary, Japan, Lithuania, Moldova, Netherlands, Poland, UK, and USA. Patients were enrolled from January 2016 to September 2017.

The study was compliant with the International Conference on Harmonisation (ICH) guideline on good clinical practice. All informed consent forms and protocols were approved by appropriate ethical review boards prior to initiation of the study. All patients gave written informed consent prior to receiving the study drug.

### Procedures and Outcomes

Endoscopic findings were scored by one of two blinded central readers. Histologic disease activity was assessed by a central reader using two biopsy samples obtained during endoscopy at baseline and Study Week 12. All biopsy specimens were collected at least 30 cm from the anal verge.

Where discrete lesions were present, biopsies were obtained preferentially at the edge of ulcers, or, if ulcers were not present, from the edge of erosions. Where visible macroscopic disease was present but without discrete lesions, biopsies were obtained spaced throughout the affected mucosa. In the absence of macroscopic disease, biopsies were obtained from throughout the segment.

### Histopathology

Two endoscopic biopsy samples for histopathological assessment were obtained from the most affected area at least 30 cm from the anal verge at each endoscopy. One of two blinded pathologists assessed histologic disease activity for each sample using the Geboes score and the Robarts Histopathology Index (RHI). The Geboes Score is comprised of seven categories (or grades), each of which describes a histologic item, including “structural (architectural change)” (grade 0), “chronic inflammatory infiltrate” (grade 1), “lamina propria eosinophils” (grade 2A), “lamina propria neutrophils” (grade 2B), “neutrophils in epithelium” (grade 3), “crypt destruction” (grade 4) and “surface epithelial injury” (grade 5). Each grade includes subscores that indicate the degree of abnormality seen for that histologic characteristic, with subscores of 0 indicating normal appearance and higher subscores indicating increasingly abnormal appearance. The RHI uses the weighted results from 4 Geboes score categories (“chronic inflammatory infiltrate”, “lamina propria neutrophils”, “neutrophils in epithelium” and “surface epithelial injury”) to derive a continuous score, ranging from 0 (no disease activity) to 33 (severe disease activity). The RHI was developed as a responsive instrument to detect treatment effects in early drug development.

Endpoints for this Example were endoscopic improvement (endoscopic subscore of 0 or 1) and histologic remission (defined as Geboes histologic subscores of 0 for the neutrophils in lamina propria, neutrophils in epithelium, and erosion or ulceration parameters). Mucosal healing was determined by the presence of both histologic remission and endoscopic improvement. As there is no agreed upon definition of what constitutes increased lamina propria eosinophils and given the lack of reproducibility and insufficient predictive data, reducing eosinophils to normal was not included in the primary definition of histologic remission. Robarts Histopathology Index (RHI) scores were determined concomitantly with Geboes scores.

### RNA Extraction and Gene Array Methodology

Gene expression was measured in 553 colonic tissue biopsies from I6T-MC-AMAC using the Affymetrix WT protocol on the GeneChip HT A2.0 arrays. Biopsies from the same subject for each time-point were pooled together to get a total of 277 biopsy RNA samples. These were subjected to Quality Control (“QC”) check points (e.g. RNA sample quality/quantity, and amplification) and proceeded to HTA 2.0 processing.

### RNA Sample Preparation and Quality Control

Colonic tissue samples arrived in two tubes per subject per time point. An extraction pilot was performed by pooling the colon biopsies from 20 subjects to ensure enough mass was available for the transcriptome analysis. The RNA extraction was performed following manufacturer-recommended protocols. Briefly, extracted RNA QC included both BioAnalyzer (BA) QC and Quantification. BA-based QC metrics were: 28S/18S Ratio (0.75-3.0), with an RNA Integrity Number (RIN) Score (≥6). RNA concentration was measured using RIBOGREEN® fluorescent dye assay. Samples with a concentration of less than 5 ng/μl were excluded from the subsequent profiling steps. Samples were run on the Agilent 2100 bioanalyzer to assess RNA quality. All samples had enough mass for assay and moved forward to HTA2 processing.

RNA samples that passed CGL QC metrics were aliquoted into 96-well plates (100 ng input). Two samples were removed after the withdrawal of one patient, and 4 biopsies from Week 12 were not collected from subjects that left the trial after their Week 0 biopsies were collected, leaving 224 Week 0 timepoint and 220 Week 12 timepoint data sets that passed array QC.

### Gene Chip Array

Probe-level data from the HTA2 platform were pre-processed with background correction and quantile normalization per standard RMA methods and summarized to the level of probesets as defined by the Affymetrix NETAFFX™ NA35/GRCh37 human reference genome release. The data were then summarized to the level of “exon-groups”, data-defined clusters of highly-correlated exon-based probesets, as follows: The correlation matrix between the probeset expressions was computed and a distance metric between pairs of probesets defined as one minus the correlation of the pair. Exon groups were formed by performing hierarchical clustering using the R hclust function and cutting the dendrogram at the 0.8 distance. Once exon groups were defined, the summarized expression for the samples were obtained from the sample effects in a two-way ANOVA model. Probe and sample effects were estimated using robust regression as implemented by the rlm function in the R MASS package applied to the probe level data within that exon group.

### Differential Expression Statistical Analyses

Exon groups generated from the procedure above were filtered according to two criteria: 1) exon groups for which the SD of the log 2(expression) was smaller than 0.286 (corresponding to roughly 20% CV), and 2) exon groups whose mean of the log 2(expression) were below the 75 percentile of median of log 2 expression for negative control (normgene->introns) probesets. In cases where a gene contained multiple exon-groups, the feature with the largest number of probesets was selected to represent the expression of the gene as a whole, with ties broken by the feature with the highest mean expression level. In all models, multiplicity corrections were applied to the resulting p-values to account for the number of comparisons made across treatment groups as well as across all tested exon-groups using the Benjamini-Hochberg method.

A mixed effect repeated measurements model (MRMM) was fit to each filtered exon-group separately to calculate fold changes between the Week 0 and Week 12 timepoints using age, sex, batch, BMI at baseline, previous biologics therapy, and Modified Mayo Score (MMS) at baseline as covariates. Crossed timepoint contrast models compared the differential expression of each exon-group in a dosed treatment group with its differential expression in the placebo group. Exon-groups with fold changes greater than 0.5 log 2 units (˜1.41× change) and false discovery rate (FDR)-adjusted q-values less than 0.05 were classified as differentially expressed.

### Correlation Statistical Analyses

Pearson correlation coefficients between the crossed-timepoint differential expression of exon-groups and the change in clinical metrics between Week 0 and Week 12 were calculated using age, sex, and array chip batch as covariates. The clinical metrics included MMS, Total Mayo Score, Mayo Endoscopic Subscore, Ulcerative Colitis Endoscopic Index of Severity (UCEIS) Total Score, Geboes Score, and Robarts Histopathology Index (RHI). The laboratory results included were fecal calprotectin and peripheral blood titers of IL-17A, IL-22, and CRP.

This study was registered with ClinicalTrials.gov, number NCT02589665.

### Results

### Patients, Study Outline and Biological Samples

Between December 2015 and September 2017, 358 patients were screened for eligibility and 249 randomized. Of these, 224 patients had a baseline biopsy, and 220 had a Week 12 biopsy. Among those 220 patients, more than 63% had previously received treatment with a biologic and 47.3% were using corticosteroids at baseline. Baseline disease characteristics were well balanced across treatment arms, with similar disease activity (Modified Mayo score), mucosal inflammation (Mayo endoscopy subscore), and histology (Geboes index and RHI) among treatment groups (Table 1).

TABLE 1
Genes with the greatest change in expression from baseline at
Week 12 in the 200 mg mirikizumab group, adjusted for placebo.

| Most decreased ^{a} | Most increased ^{a} |
|---|---|
| REG1B ⁻⁻ | ABCG2 ++ |
| MMP3 ⁻⁻ | HMGCS2 ++ |
| REG3A ⁻⁻ | AQP8 ++ |
| DUOXA2 ⁻⁻ | TMIGD1 ++ |
| SLC6A14 ⁻⁻ | GUCA2A ++ |
| DMBT1 ⁻⁻ | SLC26A2 ++ |
| MMP1 ⁻⁻ | LOC101928405 ++ |
| REG1P ⁻⁻ | SLC26A3 + |
| S100A8 ⁻⁻ | MS4A12 + |
| IL1B ⁻⁻ | UGT2A3 + |
| IGKV2D-40 ⁻⁻ | TRPM6 + |
| PI3 ⁻⁻ | NXPE4 + |
| TNIP3 ⁻ | SLC16A9 + |
| REG1A ⁻ | ADH1C + |
| IDO1 ⁻ | PCK1 + |
| DUOX2 ⁻ | CDKN2B-AS1 + |
| NOS2 ⁻ | TMEM236 + |
| MMP10 ⁻ | CD177P1 + |
| CXCL1 ⁻ | SLC17A4 + |
| PTGS2 ⁻ | ZG16 + |

^{a} Genes ordered by greatest to least magnitude of fold change
⁻ less than PBO by at least 0.5 log2 units fold;
⁻⁻ less than PBO by at least 1 log2 unit fold;
+ greater than PBO by at least 0.5 log2 units fold;
++ greater than PBO by at least 1 log2 unit fold.

Mirikizumab-mediated Changes in Transcripts in Ulcerative Colitis The 200 mg mirikizumab treatment group, in addition to demonstrating the greatest efficacy at Week 12 compared to placebo of all three treatment arms, also had the largest number of colonic biopsy genes that were differentially expressed between baseline and Week 12 (FIG. 1A). In order to better discern the effect of mirikizumab treatment from the effect of standard-of-care, the change in gene expression was evaluated between baseline and Week 12 in the 200 mg mirikizumab group both without (FIG. 1), and with (FIG. 1C) normalization to placebo. Normalization to placebo provided a higher degree of confidence in the reduced number of transcripts that are differentially regulated.

The transcripts with the greatest change at Week 12, after normalization for change in the placebo group, are shown in Table 2. The most significant increases in expression are seen for genes encoding proteins that are common in epithelial cells found in healthy colon mucosa, including AQP8, ABCG2, HMGCS2, SLC26A3, and GUCA2A, and could reflect a recovery of the epithelial lining and barrier integrity. These robustly upregulated transcripts represent both structural components seen in a healthy colon mucosa and critical functional proteins, which may provide some evidence of return to function in these patients. The most upregulated transcript was AQP8 (aquaporin-8), which codes for a critical water transporter in the apical colon surface that mediates retrieval of water from fecal content; this may contribute a beneficial effect to patients experiencing watery and frequent diarrhea. Similarly, efflux transporters such as ABCG2 are reduced in UC and, when normalized, offer improved mucosal function in UC patients. Conversely, the greatest decreases in expression are observed in genes that have been associated with IBD activity by their functions in tissue remodeling (MMP3, MMP10), oxidative stress (DUOX2, DUOXA2, NOS2), and chemotaxis (CXCL1, CXCL2, CXCL3).

Correlation between IL-23 Pathway Transcripts and Disease Activity Scores Changes in expression levels of genes that are upregulated by IL-23 receptor activation, as defined in the METACORE™ (Clarivate, Philadelphia, USA) database, were correlated with changes disease activity metrics MMS, UCEIS, and RHI at Week 12 (FIG. 2A). The most highly correlated genes include TGFB1, IL1B, and TL6. Of these, IL1B is also one of the genes most reduced in expression between baseline and Week 12 in the placebo-adjusted 200 mg mirikizumab group. All the genes upregulated by IL-23/IL-23R engagement and are significantly correlated with changes in disease activity metrics have positive correlations, with degree of gene expression change correlating with improved disease activity metrics, with the exception of IL22 which did not pass the FDR ≤0.05 significance threshold. FIG. 2B presents a heatmap of Pearson's correlation coefficients (Rho) for the placebo-adjusted differential expression of each gene between baseline and Week 12 with the change over the same period of modified Mayo score, RHI, and UCEIS sorted vertically by the fold changes of these genes. Mirikizumab-regulated genes correlate more strongly with the disease indices for histopathology (MMS, RHI) than with endoscopy (UCEIS), which may provide a more nuanced view of these transcripts. We see IL1B having both high correlations to disease activity and high differential expression relative to the other genes.

### Correlation Between Transcripts Changed by Mirikizumab and Disease Activity Scores

Changes in individual transcripts correlated with changes in disease activity as defined by MMS, Geboes or RHI. Several transcripts which reflect the most robust changes (Table 2) in mirikizumab treatment-induced regulation identify with changes in disease activity, and all the transcripts which positively correlate with disease are uniformly and consistently down regulated by mirikizumab. In contrast, all transcripts that negatively associate with disease are upregulated with mirikizumab treatment. This is consistent with the observation that changes in mirikizumab-regulated transcripts demonstrate a profile of attenuation of disease and suggests preliminary evidence of molecular healing as early as 12 weeks after initiation of treatment.

Pathway Analysis of Top Mirikizumab-Regulated Genes Most Highly Correlated with Change in Disease Scores

The most highly modulated of the transcripts identified in FIG. 3, with the stringent cut-off of r values greater than ±0.4, were further analyzed. The two disease indices chosen were RHI and MMS, which offered the highest −log₁₀ q values for these correlations. As shown in FIG. 4 and Table 2, these transcripts provided a glimpse into the common pathways that are altered with change in disease activity and include cell adhesion and extracellular matrix (ECM) remodeling, followed by features seen in pro-fibrotic pathways and tissue repair.

Correlation of Mirikizumab-Induced Changes in Gene Expression with Systemic Biomarkers

Correlation between transcripts for IL-18, IL-6, S100A8 and S100A9 to systemic biomarkers IL-17, IL-22, C-reactive protein (CRP) and fecal calprotectin (fCLP) (Table 2) was determined. Changes in colonic expression of S100A8/9, IL-1B, and IL-6 strongly correlated with levels of fCLP as well as serum IL-17A, and less strongly, yet still significantly, with serum CRP levels. Circulating levels of IL-22 were significantly correlated with changes in S100A9 and IL-1B, but not S100A8 or IL-6. These correlations indicated that accessible biomarkers such as fCAL, IL-1B and IL-6 are correlated with the changes in some of the transcripts encoding these proteins and reflect p19 engagement in the colonic mucosa.

TABLE 2
Correlation matrix of protein biomarkers with change
from baseline in expression of key transcripts.

| | Protein | | | | | | | |
|---|---|---|---|---|---|---|---|---|
| | CRP | | fCLP | | IL-17A | | IL-22 | |
| | P | | P | | P | | P | |
| Gene | value | Rho | value | Rho | value | Rho | value | Rho |
| S100A8 | 0.012 | 0.345 | <0.001 | 0.515 | <0.001 | 0.505 | 0.120 | 0.235 |
| S100A9 | 0.008 | 0.362 | <0.001 | 0.494 | <0.001 | 0.534 | 0.044 | 0.301 |
| IL1B | 0.004 | 0.389 | 0.009 | 0.377 | <0.001 | 0.545 | 0.033 | 0.319 |
| IL6 | 0.016 | 0.332 | 0.005 | 0.399 | <0.001 | 0.499 | 0.059 | 0.283 |

Pearsons' correlation of change from baseline in gene expression with log2(protein) levels from the residuals of the Linear Mixed effects Models that each adjust for batch, age and sex.

### Mirikizumab Modulated Core TNF Resistance Transcripts

Smillie et al. reported that inflammation-associated fibroblasts, monocytes, and dendritic cells showed a high relative expression of genes that were part of a drug resistance signature identified using a meta-analysis of bulk expression data from 60 responders and 57 non-responders to anti-TNFα therapy. In contrast to the drug resistant phenotype, the anti-TNFα sensitive genes in the colon tissue were most enriched in epithelial cells. FIG. 5A shows the transcripts included in the meta-analysis that reflect key drivers of TNF^{R}. The number of transcripts regulated by mirikizumab in the TNF-sensitive cluster (FIG. 5B) were fewer than those seen in the TNF^{R} cluster (FIG. 5A). This result is consistent with the distinct mechanism of action for mirikizumab that targets the p19-IL23 protein compared to antibodies that target TNFα. Mirikizumab regulated genes that are pre-dominant in the TNF^{R} transcript cluster. Collectively, these results indicated that mirikizumab treatment may serve to reduce the transcripts associated with TNF^{R} and may offer a different phenotype in the colonic tissue than those seen in TNF^{R} patients.

Robust changes in transcript expression levels were observed at the 200 mg mirikizumab group. These changes provide a window into the changes elicited by mirikizumab treatment. The genes upregulated by mirikizumab indicate a trend towards healthy mucosa, with the top regulated transcripts representing improvements in barrier integrity and those that provide functional transporters in the colon; for example, the increase in Aquaporin 8, a water transporter, may limit the water content in fecal matter. Increases in anion transporters SLC26A2 and SLC16A9 reaffirm that mirikizumab treatment may lead to a mucosa capable of retaining functional transporters as early 12 weeks after initiation of treatment. Some of the genes most robustly downregulated by mirikizumab, such as REG1A and REG1B, have been shown to be increased in UC in previous studies conducted in UC mucosal biopsies.

Of relevance is the change in the S100A8 and A9 transcripts which contribute to the calprotectin protein derived from the colonic mucosa. It has been previously reported that mirikizumab treatment leads to dose-dependent changes in fCLP; these changes may be a result of reductions in calprotectin transcripts derived from the colon mucosa. Consistent with this finding, a statistically significant correlation was observed with the decreased levels of fCLP in those patients in whom the S100A8 and S100A9 transcripts were downregulated in the colon with mirikizumab treatment. The most highly mirikizumab-regulated genes reflect a reduction in inflammatory signals (IL1B, CXCL1, CXCL2, and CXCL3), attenuation of the transcripts that mediate matrix disruption of the colonic mucosa (matrix metalloproteases 10 and 3), and an increase in anion and water transporters. These molecular changes indicate initiation of mucosal healing.

The IL23 pathway has been implicated in genetic predisposition for UC, confirmed by a meta-analysis of the association of ten polymorphisms in the IL23R gene (excepting rs10489629) with UC risk. Analysis of IL23-regulated transcripts overlaid onto the change in transcripts correlated with disease activity indicated a consistent downregulation of IL23 pathway genes. Analysis of UC patients demonstrated that inflammation-associated fibroblasts, monocytes and tissue-resident antigen presenting cells exhibited high relative expression of genes that are enriched in biopsy samples from patients who represent primary and secondary failures from anti-TNF therapies. In contrast, the anti-TNFα sensitive genes in the colon tissue originated from epithelial cells. In a meta-analysis of the transcripts that are enriched in such TNF^{R}colon mucosa, several transcripts shown in Table 2 as being regulated by mirikizumab appeared. These transcripts are potentially represented in those cell types identified by the previous studies. Of importance is the change in OSMR expression, which was downregulated by mirikizumab. It has been previously hypothesized that OSM represents a potential alternate disease pathway of divergence as the colonic mucosa acquires TNF^{R}. OSM, a gene showing significantly increased expression in the mucosa of TNF^{R} UC patients compared to anti-TNF responsive patients, was enriched in inflammation-associated monocytes and antigen presenting cells whereas its receptor OSMR was enriched in inflammation-associated fibroblasts. The finding that mirikizumab downregulated OSMR in the colon mucosa within 12 weeks of mirikizumab treatment indicates a suppression of this pathway's activation seen in TNF^{R} patients, which may result in a better outcome for such patients. Moreover, a number of genes associated with OSM activity are regulated by mirikizumab.

The changes observed within 12 weeks of mirikizumab treatment were distinct from those observed after vedolizumab (VDZ) treatment. However, the vedolizumab analysis of the transcriptome changes were not normalized for changes to their placebo group, which it is believed offers a more stringent method. This may have resulted in some transcript changes that associated to the edge of the q=0.05 threshold. As such, it is with greater confidence that the gene expression changes observed in the placebo-adjusted 200 mg mirikizumab group are associated with mirikizumab treatment rather than being associated with spontaneous mucosal healing. There was very little in common between the top 10 transcripts altered by VDZ vs the control group and the top 10 transcripts identified in the Examples, potentially because the comparison for VDZ was to healthy controls instead of diseased baseline patients enrolled in the VDZ trial. Notably, changes in expression at Week 12 relative to Week 0 appeared to be modest. Changes in expression at Week 52 after treatment with VDZ displayed some overlap with Week 12 changes with mirikizumab. A majority of mirikizumab induced changes were clustered within the signaling pathways derived from the Th17 pathway, while that with VDZ were derived from chemotaxis, consistent with the mechanism of action of VDZ which targets the a4B7 integrin and the resulting chemotactic signals.

### Example 2

### Methods:

Mirikizumab-treated patients who achieved clinical response (decrease in 9-point Mayo subscore [rectal bleeding, stool frequency, endoscopy] of ≥2 points and ≥35% from baseline [BL] with either a decrease of RB subscore of ≥1 or an RB subscore of 0 or 1) or better at week 12 were re-randomized to mirikizumab 200 mg administered subcutaneously (SQ) every 4 weeks or every 12 weeks through week 52. Patients given placebo (PBO) in induction who achieved clinical response continued on PBO in the maintenance period. Colonic biopsies were obtained at weeks 0, 12, and 52 from the most affected area at least 30 cm from the anal verge (mirikizumab N=31, PBO N=7). Transcript changes at week 12 from baseline in the PBO and mirikizumab arms were clustered into differentially expressed genes (DEGs) using the Bayesian Limma R-package. Among these DEGs, similarly expressed genes (SEGs) were identified as those that maintained their week 12 expression level through week 52.

### Results:

Analysis of transcript changes at week 52 in those week 12 responders who maintained disease remission, identified a profile of DEG-SEGs in responders (see, FIG. 6). One cluster of upregulated genes at Baseline included CXCL8, AQP9, IL1B, S100A9, TREM1, MMP12, MMP1, MMP7, and TCN1. A second cluster of upregulated genes at Baseline included DUOX2, DUOXA2, SLC6A14, VNN1, ABCA12, REG1B, and C4BPA. A cluster of downregulated genes included GUCA2B, OTOP2, AQP8, SLC26A2, and ADH1C. Of these genes, 63 (70.8%) were present only in mirikizumab responders, 5 (5.6%) were present only in PBO responders, and 21 (23.6%) were present in both groups (see, FIG. 7). The magnitude of transcript changes was greater at week 12, and more consistent through week 52, in the group of mirikizumab responders compared to PBO responders. The table in FIG. 8 depicts the top ten mirikizumab-specific DEG-SEG genes. A separate cluster of DEG-SEGs correlated with disease activity indices (Robarts Histopathology Index [RHI], and modified Mayo; both r>0.5) were shown to be sustained in the mirikizumab treated patients but not in the placebo patients. At week 12 following mirikizumab treatment, the two clusters of upregulated genes at Baseline changed to downregulated and the cluster of downregulated genes at Baseline changed to upregulated. As shown in FIG. 9A, clusters of genes remained changed from weeks 12 to week 52. From week 12 to week 52, expression trended in a downward direction (see, FIG. 9 arrow direction). In placebo patient responders (“PBO”; those who showed spontaneous improvement in the absence of mirikizumab treatment), the two clusters of upregulated genes at Baseline changed to downregulated (FIG. 9C), but the cluster of downregulated genes at Baseline remained unchanged. As depicted in FIG. 9D, the biomarker changes in PBO remained constant (illustrated by horizontally oriented arrows). As depicted in FIGS. 10A-10D, the cluster of mirikizumab-responsive genes correlated with disease activity indices (RHI and Mayo). These results demonstrate that mirikizumab treatment changed the expression of the two clusters of upregulated genes at Baseline to downregulated status and changed the expression of the cluster of downregulated genes at Baseline to upregulated status. The change in the first and second gene clusters observed in the placebo responders likely reflects a response of these gene clusters to lifestyle changes made by the placebo group. As noted, the two clusters include markers involved with inflammation. In the mirikizumab group, the cluster of downregulated genes at Baseline changed to upregulated. Significantly, in the placebo group, the cluster of downregulated genes at Baseline remained downregulated. At week 52 following mirikizumab treatment, the two clusters of upregulated genes at Baseline remained downregulated and the cluster of downregulated genes at Baseline remained upregulated. These results demonstrate that gene expression changes induced by mirikizumab during induction were maintained over time up to 52 weeks (the data collection endpoint). These results also demonstrate that mirikizumab-treated responders have differentially expressed transcripts that are quantitatively distinct in both fold change and significance from those in the PBO responders group.

In this sample of week 12 PBO and mirikizumab responders, mirikizumab responders showed broader, larger, and more sustained magnitude of changes at week 52 as compared to PBO responders. The qualitative description of transcripts indicated a distinct molecular healing pathway associated with mirikizumab treatment, as compared to the spontaneous healing that occurred in PBO responders. A cluster of transcripts that correlated with disease activity indices was identified, demonstrating consistency across molecular, endoscopic and clinical indices of mirikizumab-mediated healing in ulcerative colitis. Most up-regulated proteins were related to water and ion transports that indicates epithelial origin.

### Example 3

In this Example, expression of colonic mucosa genes and stool frequency (SF), a symptom reflective of disease activity, during the 12-week induction period of a Phase 2 study of patients with moderately to severely active UC (NCT02589665) was investigated. Mirikizumab-induced upregulation of colonic transporter transcripts improved stool frequency in a Phase 2 study of patients with moderate to severe ulcerative colitis.

### Methods:

Patients were randomized 1:1:1:1 to receive intravenous placebo (PBO), mirikizumab 50 mg or 200 mg with possibility of exposure-based dose increases, or fixed mirikizumab 600 mg every 4 weeks for 12 weeks. SF was reported daily by patients and transformed on a 4-level ordinal scale [0-3] representing increased SF above their normal or healthy baseline (BL) (SF frequency ordinal scale: 0=normal number of stools for subject; 1=1 to 2 stools more than normal; 2=3 to 4 stools more than normal; 3=5 or more stools than normal). Patient colonic biopsies (PBO N=58, mirikizumab 50 mg N=52, 200 mg N=51, 600 mg N=54) were collected at BL and Week 12, and gene expression measured using an Affymetrix HTA2.0 microarray workflow. BL and Week 12 gene expression or SF values were pooled and associations identified based on non-parametric Kendall's tau. Pathway analysis (Hallmark and Reactome) of associated genes was performed using over-representation analysis. p values of enrichment were determined by hypergeometric distribution test and adjusted for multi-testing with Benjamini-Hochberg procedure. Differential gene expression after mirikizumab treatment was determined by paired t-test comparing expression levels at BL and at Week 12using data from the 200 mg treatment group. A “positive” correlation to gene expression was considered if higher expression lead to higher SF.

A total of 267 genes were correlated with SF (|tau|>0.3 and qval <0.001; see Table 8). Of these, 212 were positively associated (high expression associated with high SF) and 55 were negatively associated (high expression associated with low SF). The 212 transcripts that were positively associated with SF were uniformly and consistently downregulated with mirikizumab treatment, while the 55 transcripts that negatively associated with SF, were consistently upregulated with mirikizumab treatment (Table 3; see also, FIG. 17). Biological pathways significantly associated with the mirikizumab-responsive transcripts that associated with SF included inflammatory response, extracellular matrix dysregulation, neutrophil degranulation and cytokine signaling pathways, particularly TNF and IL6 pathways (Table 4).

TABLE 3
Effect of mirikizumab treatment on genes
correlated with Stool Frequency.

| Genes correlated to Stool | Mirikizumab effect on expression at |
|---|---|
| Frequency | 12 Weeks |
| 212+ | 212 ↓ |
| 55 − | 55 ↑ |

Number of genes correlated to SF: [tau] >0.3 and qval <0.001
212 were positively correlated (+) and 55 negatively correlated (−).
The effect of mirikizumab treatment on these 267 genes indicated with (↓) downregulated or (↑) upregulated.

TABLE 4
Pathway analysis of the 267 genes
associated with Stool Frequency.

| | | Adjusted |
|---|---|---|
| Collection | Pathway | P value |
| Hallmark | Inflammatory Response | 1.60E−12 |
| | TNFα Signaling via NFKB | 1.51E−09 |
| | Epithelial-Mesenchymal Transition | 4.89E−05 |
| | Complement | 0.000163 |
| | Coagulation | 0.00026 |
| | Interferon Gamma Response | 0.001143 |
| | KRAS Signaling Up | 0.002451 |
| | Allograft Rejection | 0.007556 |
| | IL6-JAK STAT3 Signaling | 0.008698 |
| Reactome | Neutrophil Degranulation | 4.68E−14 |
| | Signaling by Interleukins | 8.14E−06 |
| | Toll-like Receptor Cascades | 7.28E−05 |
| | Extracellular Matrix Organization | 0.000207 |
| | Cell Surface Interactions at the | 0.002191 |
| | Vascular Wall | |
| | Platelet Activation Signaling and | 0.002409 |
| | Aggregation | |

Pathway analysis of the 267 genes correlated with stool frequency using over representation analysis (ORA) on Hallmark and Reactome gene sets from MSigDB with clusterProfilder R. Package.
Pathways with adjusted p values <0.01 were selected and redundant pathways removed. Pathways were determined to be redundant when over 80% of hit genes in a pathway overlap with genes from another pathway with lower adjusted p values.

The top 20 genes associated with SF are listed in Table 5. FIG. 13 shows tau distribution for the top 20 genes associated with SF. FIG. 14 shows the expression of S100A8, MMP3, AQP9, and CDHR1 in relation to MMS-SF scores.

TABLE 5
Top 20 Genes Associated with SF.

| Gene Symbol | Name | tau |
|---|---|---|
| S100A8 | S100 calcium binding protein 8 | ↑ 0.379 |
| S100A12 | S100 calcium binding protein A12 | ↑ 0.371 |
| CDHR1 | Cadherin related family member 1 | ↓ −0.37 |
| S100A9 | S100 calcium binding protein A9 | ↑ 0.363 |
| TRIB2 | Tribbles pseudokinase 2 | ↑ 0.36 |
| PTAFR | Platelet activating factor receptor | ↑ 0.359 |
| AIFM3 | Apoptosis inducing factor | ↓ −0.359 |
| | mitochondria associated 3 | |
| FCGR3B | Fc fragment of IgG receptor IIb | ↑ 0.357 |
| CSF3R | Colony stimulating factor 3 receptor | ↑ 0.356 |
| LYN | LYN proto-oncogene, Src family | ↑ 0.356 |
| | tyrosine kinase | |
| IFITM2 | Interferon induced transmembrane | ↑ 0.356 |
| | protein 2 | |
| CAPN13 | Calpain 13 | ↓ −0.356 |
| ELL2 | Elongation factor for RNA | ↑ 0.356 |
| | polymerase II 2 | |
| PROK2 | Prokineficin 2 | ↑ 0.355 |
| AQP9 | Aquaporin 9 | ↑ 0.355 |
| IL1A | Interleukin 1 alpha | ↑0.355 |
| FCGR2A | Fc fragment of IgG receptor IIa | ↑ 0.354 |
| TIMP1 | TIMP metallopeptidase inhibitor 1 | ↑ 0.354 |
| TCN1 | Transcobalamin 1 | ↑ 0.353 |
| CKB | Creatine kinase B | ↓ 0.353 |

This Example identified colon-based transcripts that associate with a clinical disease activity measure, stool frequency, and demonstrates that treatment with mirikizumab upregulated genes associated with normalization of SF and down regulated genes associated with inflammation in colonic tissue samples of patients with UC.

### Example 4

In this Example, the association between colon tissue transcripts and patient-reported bowel urgency (BU) at Baseline (BL) and Week (W)12 was investigated and the effect of mirikizumab induction treatment on genes associated with BU was assessed.

Patients were randomized 1:1:1:1 to receive intravenous placebo (N=63), mirikizumab 50 mg (N=63) or 200 mg (N=62) with possibility of exposure-based dose increases, or fixed mirikizumab 600 mg (N=61) every 4 weeks for 12 weeks. Patient colonic biopsies were collected at BL and Week 12 (placebo N=58, miri 50 mg N=52, 200 mg N=51, 600 mg N=54). Gene expression was measured using an Affymetrix HTA2.0 microarray workflow. Differential gene expression was determined by paired T-test comparing expression values at Week 12 and BL. BU was reported daily by patients as yes/no. Proportion of days of BU at BL and at Week 12 was calculated for the 3 days prior to visit on a 4-point ordinal scale (0=absence of urgency on all 3 prior days; 3=presence of urgency all 3 days). BL and Week 12 gene expression and BU values were pooled and associations identified based on non-parametric Kendall's tau. Pathway analysis of correlated genes were performed using over-representation analysis on Hallmark and Reactome gene sets from MSigDB. p values of enrichment were determined by hypergeometric distribution test and adjusted for multi-testing with Benjamini-Hochberg (BH) procedure. A “positive” correlation to gene expression was considered if higher expression lead to more BU.

A total of 249 patients reported BU scores at BL and Week 12. The presence of BU was associated with 320 genes (|tau|>0.225 and val <0.001). Pathway analysis identified pathways significantly associated with 0U (Table 6). Among the 320 correlated transcripts, 296 were positively associated (higher gene expression) and 24 were negatively associated (lower gene expression) with the frequency of BU (see, FIG. 17). Treatment with mirikizumab (200 mg) resulted in a statistically significant decrease in the 296 transcripts positively associated with BU, and an increase in the 24 transcripts negatively correlated with BF.

TABLE 6
Pathways analysis of the 267 genes associated with bowel urgency.

| Collection | Pathway | P value* |
|---|---|---|
| Hallmark | Interferon Gamma Response | 1.33E−06 |
| | Complement | 1.62E−05 |
| | TNFA Signaling via KFKB | 0.0002 |
| | Allograft rejection | 0.0013 |
| | MTORC1 Signaling | 0.0034 |
| | Apoptosis | 0.009 |
| Reactome | FCGR Activation | 0.0016 |
| | FCERI Mediated CA 2 | 0.0027 |
| | mobilization | |
| | MHC Class II Antigen | 0.0027 |
| | Presentation | |
| | Toll like Receptor Cascades | 0.0027 |
| | Tryptophan Catabolism | 0.0033 |
| | FCGamma Receptor FCGR | 0.0051 |
| | Dependent Phagocytosis | |
| | Neutrophil Degranulation | 0.0054 |
| | Leishmania Infection | 0.0056 |
| | Interferon Signaling | 0.0063 |

Pathway analysis of the 320 genes
*BH adjusted

The top 20 genes associated with BU are listed in Table 7.

FIG. 15 shows tau distribution for the top 20 genes associated with SF.

FIG. 16 shows the expression of S100A8, MMP3, AQP9, and CDHR1 in relation to MMS-SF scores.

TABLE 7
Top 20 Genes Associated with BU.

| Gene Symbol | Name | tau |
|---|---|---|
| CCDC175 | Coiled-coil domain containing 175 | ↓ 0.297 |
| TNFRSF17 | TNF receptor superfamily member 17 | ↑ 0.277 |
| CFB | Complement factor B | ↑ 0.273 |
| FBXW7 | F-box and WD repeat domain | ↑ 0.271 |
| | containing 7 | |
| LIPA | Lipase A, lysosomal acid type | ↑ 0.269 |
| CEP128 | Centrosomal protein 128 | ↑ 0.268 |
| BIRC3 | Baculoviral IAP repeat containing 3 | ↑ 0.267 |
| IFNAR2 | Interferon alpha and beta receptor | ↑ 0.267 |
| | subunit 2 | |
| PSAT1 | Phosphoserine aminotransferase 1 | ↑ 0.267 |
| SNX25 | Sortin nexin 25 | ↑ 0.266 |
| HSPA13 | Heat shock protein family A (Hsp70) | ↑ 0.263 |
| | member 13 | |
| CLDN2 | Claudin 2 | ↑ 0.263 |
| LY96 | Lymphocyte antigen 96 | ↑ 0.262 |
| SEC11C | SEC11 homolog C, signal peptidase | ↑ 0.261 |
| | complex subunit | |
| DRAM1 | DNA damage regulated autophagy | ↑ 0.26 |
| | modulator 1 | |
| CPEB4 | Cytoplasmic polyadenylation element | ↑ 0.26 |
| | binding protein 4 | |
| PCK1 | Phosphoenolpyruvate carboxykinase 1 | ↓ 0.259 |
| ELL2 | Elongation factor for RNA polymerase | ↑ 0.259 |
| | II 2 | |
| CTSH | Cathepsin H | ↑ 0.259 |
| CAPN13 | Calpain 13 | ↓ 0.259 |

This Example identified colon-based transcripts pertinent to mucosal inflammation and healing that correlated with bowel urgency and that are consistently modulated by mirikizumab.

TABLE 8
Genes associated with SF and modulated by anti-IL-23p19 antibody treatment.

| Symbol | Name | tau |
|---|---|---|
| CDHR1 | cadherin related family member 1 | −0.370 |
| AIFM3 | apoptosis inducing factor mitochondria associated 3 | −0.359 |
| CAPN13 | calpain 13 | −0.356 |
| CKB | creatine kinase B | −0.353 |
| EXPH5 | exophilin 5 | −0.352 |
| ZNF704 | zinc finger protein 704 | −0.348 |
| CCDC175 | coiled-coil domain containing 175 | −0.347 |
| WNK4 | WNK lysine deficient protein kinase 4 | −0.346 |
| CPA6 | carboxypeptidase A6 | −0.340 |
| NXPE4 | neurexophilin and PC-esterase domain family member 4 | −0.338 |
| CWH43 | cell wall biogenesis 43 C-terminal homolog | −0.333 |
| TRPM6 | transient receptor potential cation channel subfamily M | −0.332 |
| | member 6 | |
| PCK1 | phosphoenolpyruvate carboxykinase 1 | −0.331 |
| ACSF2 | acyl-CoA synthetase family member 2 | −0.330 |
| PADI2 | peptidyl arginine deiminase 2 | −0.329 |
| VIPR1 | vasoactive intestinal peptide receptor 1 | −0.329 |
| LOC285423 | | −0.328 |
| GUCA2A | guanylate cyclase activator 2A | −0.326 |
| LOC101929718 | uncharacterized LOC101929718 | −0.325 |
| TRABD2A | TraB domain containing 2A | −0.324 |
| TCEA3 | transcription elongation factor A3 | −0.324 |
| SATB2 | SATB homeobox 2 | −0.322 |
| FAAH | fatty acid amide hydrolase | −0.322 |
| SAMD13 | sterile alpha motif domain containing 13 | −0.321 |
| DPF3 | double PHD fingers 3 | −0.320 |
| SLC26A2 | solute carrier family 26 member 2 | −0.318 |
| SLC4A4 | solute carrier family 4 member 4 | −0.316 |
| C7orf13 | | −0.316 |
| PMFBP1 | polyamine modulated factor 1 binding protein 1 | −0.314 |
| ANO7 | anoctamin 7 | −0.314 |
| PLCD1 | phospholipase C delta 1 | −0.314 |
| MROH7 | maestro heat like repeat family member 7 | −0.313 |
| LOC145837 | | −0.313 |
| FOXD2-AS1 | FOXD2 adjacent opposite strand RNA 1 | −0.312 |
| HOXA11-AS | HOXA11 antisense RNA | −0.312 |
| PDE6A | phosphodiesterase 6A | −0.312 |
| HEPACAM2 | HEPACAM family member 2 | −0.311 |
| IHH | Indian hedgehog signaling molecule | −0.310 |
| C21orf88 | | −0.310 |
| OTOP2 | otopetrin 2 | −0.310 |
| SLC9A2 | solute carrier family 9 member A2 | −0.309 |
| SATB2-AS1 | SATB2 antisense RNA 1 | −0.308 |
| CHP2 | calcineurin like EF-hand protein 2 | −0.308 |
| VSTM2A | V-set and transmembrane domain containing 2A | −0.307 |
| MFSD4 | | −0.307 |
| FRMD1 | FERM domain containing 1 | −0.305 |
| RETNLB | resistin like beta | −0.305 |
| WNK2 | WNK lysine deficient protein kinase 2 | −0.304 |
| PXMP2 | peroxisomal membrane protein 2 | −0.304 |
| CA1 | carbonic anhydrase 1 | −0.303 |
| ZG16 | zymogen granule protein 16 | −0.302 |
| PPARGC1A | PPARG coactivator 1 alpha | −0.302 |
| ARHGAP44 | Rho GTPase activating protein 44 | −0.301 |
| CDKN2B-AS1 | CDKN2B antisense RNA 1 | −0.300 |
| SYTL2 | synaptotagmin like 2 | −0.300 |
| PCDH17 | protocadherin 17 | 0.300 |
| KLK10 | kallikrein related peptidase 10 | 0.300 |
| TGFBI | transforming growth factor beta induced | 0.300 |
| IFI30 | IFI30 lysosomal thiol reductase | 0.300 |
| TDO2 | tryptophan 2,3-dioxygenase | 0.300 |
| PLA1A | phospholipase Al member A | 0.300 |
| PLXNA1 | plexin A1 | 0.300 |
| CEP170 | centrosomal protein 170 | 0.301 |
| BCL2A1 | BCL2 related protein A1 | 0.301 |
| GNAI2 | G protein subunit alpha i2 | 0.301 |
| MMP14 | matrix metallopeptidase 14 | 0.301 |
| SOD2 | superoxide dismutase 2 | 0.301 |
| ITGAV | integrin subunit alpha V | 0.301 |
| BCAT1 | branched chain amino acid transaminase 1 | 0.301 |
| SLA | Src like adaptor | 0.302 |
| CHI3L1 | chitinase 3 like 1 | 0.302 |
| ACSL1 | acyl-CoA synthetase long chain family member 1 | 0.302 |
| DUOXA2 | dual oxidase maturation factor 2 | 0.303 |
| PDLIM7 | PDZ and LIM domain 7 | 0.303 |
| TLR1 | toll like receptor 1 | 0.303 |
| GLT1D1 | glycosyltransferase 1 domain containing 1 | 0.303 |
| LAX1 | lymphocyte transmembrane adaptor 1 | 0.303 |
| PHTF1 | putative homeodomain transcription factor 1 | 0.304 |
| RAPGEF4 | Rap guanine nucleotide exchange factor 4 | 0.304 |
| CDH3 | cadherin 3 | 0.304 |
| IRAK3 | interleukin 1 receptor associated kinase 3 | 0.304 |
| CTSB | cathepsin B | 0.304 |
| GNS | glucosamine (N-acetyl)-6-sulfatase | 0.304 |
| FFAR2 | free fatty acid receptor 2 | 0.304 |
| AHR | aryl hydrocarbon receptor | 0.305 |
| FSTL1 | follistatin like 1 | 0.305 |
| LOC101928136 | | 0.305 |
| BIRC3 | baculoviral IAP repeat containing 3 | 0.305 |
| INHBA | inhibin subunit beta A | 0.305 |
| SLC6A14 | solute carrier family 6 member 14 | 0.305 |
| DUOX2 | dual oxidase 2 | 0.305 |
| AAED1 | | 0.305 |
| MSN | moesin | 0.305 |
| ENTPD1 | ectonucleoside triphosphate diphosphohydrolase 1 | 0.306 |
| FBXW7 | F-box and WD repeat domain containing 7 | 0.306 |
| CD44 | CD44 molecule (Indian blood group) | 0.306 |
| KRT6A | keratin 6A | 0.306 |
| FAM129A | | 0.306 |
| SH3PXD2B | SH3 and PX domains 2B | 0.306 |
| SLC4A11 | solute carrier family 4 member 11 | 0.306 |
| PTP4A3 | protein tyrosine phosphatase 4A3 | 0.306 |
| ITGA5 | integrin subunit alpha 5 | 0.307 |
| SERPINB9 | serpin family B member 9 | 0.307 |
| SELE | selectin E | 0.307 |
| EMR2 | | 0.308 |
| RNF149 | ring finger protein 149 | 0.308 |
| TFPI | tissue factor pathway inhibitor | 0.309 |
| TRAM1 | translocation associated membrane protein 1 | 0.309 |
| OLFML2B | olfactomedin like 2B | 0.309 |
| CEP128 | centrosomal protein 128 | 0.309 |
| ELTD1 | | 0.309 |
| KRT6B | keratin 6B | 0.310 |
| ELOVL5 | ELOVL fatty acid elongase 5 | 0.310 |
| SEC14L1 | SEC14 like lipid binding 1 | 0.310 |
| CTSL | cathepsin L | 0.310 |
| TLR8 | toll like receptor 8 | 0.310 |
| WNT5A | Wnt family member 5A | 0.310 |
| NNMT | nicotinamide N-methyltransferase | 0.310 |
| CLEC7A | C-type lectin domain containing 7A | 0.310 |
| STRIP2 | striatin interacting protein 2 | 0.310 |
| OSBPL3 | oxysterol binding protein like 3 | 0.310 |
| CHST15 | carbohydrate sulfotransferase 15 | 0.311 |
| NCOA7 | nuclear receptor coactivator 7 | 0.311 |
| RAB31 | RAB31, member RAS oncogene family | 0.312 |
| SERPINI1 | serpin family I member 1 | 0.312 |
| KCNN3 | potassium calcium-activated channel subfamily N | 0.312 |
| | member 3 | |
| ITGAX | integrin subunit alpha X | 0.312 |
| ACVR1 | activin A receptor type 1 | 0.312 |
| SMOX | spermine oxidase | 0.312 |
| NAMPT | nicotinamide phosphoribosyltransferase | 0.313 |
| FCAR | Fc fragment of IgA receptor | 0.313 |
| CSF2RB | colony stimulating factor 2 receptor subunit beta | 0.313 |
| DNAJB9 | DnaJ heat shock protein family (Hsp40) member B9 | 0.313 |
| SLC11A1 | solute carrier family 11 member 1 | 0.313 |
| TAGAP | T cell activation RhoGTPase activating protein | 0.313 |
| SPP1 | secreted phosphoprotein 1 | 0.313 |
| PDZK1IP1 | PDZK1 interacting protein 1 | 0.313 |
| IFITM1 | interferon induced transmembrane protein 1 | 0.314 |
| SLC2A3 | solute carrier family 2 member 3 | 0.314 |
| TTYH3 | tweety family member 3 | 0.314 |
| IL1RN | interleukin 1 receptor antagonist | 0.315 |
| DRAM1 | DNA damage regulated autophagy modulator 1 | 0.315 |
| RIPK2 | receptor interacting serine/threonine kinase 2 | 0.315 |
| CTSK | cathepsin K | 0.316 |
| FCGR2B | Fc fragment of IgG receptor IIb | 0.316 |
| DUSP4 | dual specificity phosphatase 4 | 0.316 |
| IL1B | interleukin 1 beta | 0.316 |
| LILRA1 | leukocyte immunoglobulin like receptor A1 | 0.316 |
| CTSH | cathepsin H | 0.316 |
| TNC | tenascin C | 0.317 |
| CALU | calumenin | 0.317 |
| ACSL4 | acyl-CoA synthetase long chain family member 4 | 0.317 |
| CHSY1 | chondroitin sulfate synthase 1 | 0.317 |
| MGP | matrix Gla protein | 0.317 |
| LY96 | lymphocyte antigen 96 | 0.318 |
| MMP10 | matrix metallopeptidase 10 | 0.318 |
| TNFAIP6 | TNF alpha induced protein 6 | 0.318 |
| DEGS1 | delta 4-desaturase, sphingolipid 1 | 0.318 |
| ARFGAP3 | ADP ribosylation factor GTPase activating protein 3 | 0.318 |
| DCC | DCC netrin 1 receptor | 0.318 |
| IFNAR2 | interferon alpha and beta receptor subunit 2 | 0.318 |
| ARNTL2 | aryl hydrocarbon receptor nuclear translocator like 2 | 0.318 |
| LILRB2 | leukocyte immunoglobulin like receptor B2 | 0.320 |
| RHOQ | ras homolog family member Q | 0.320 |
| DDIT4 | DNA damage inducible transcript 4 | 0.320 |
| MB21D1 | | 0.320 |
| THEMIS2 | thymocyte selection associated family member 2 | 0.321 |
| STEAP4 | STEAP4 metalloreductase | 0.321 |
| STOM | stomatin | 0.322 |
| XBP1 | X-box binding protein 1 | 0.322 |
| MLKL | mixed lineage kinase domain like pseudokinase | 0.322 |
| PHLDA1 | pleckstrin homology like domain family A member 1 | 0.322 |
| FCER1G | Fc fragment of IgE receptor Ig | 0.322 |
| PRDX4 | peroxiredoxin 4 | 0.322 |
| CLEC4E | C-type lectin domain family 4 member E | 0.322 |
| PECAM1 | platelet and endothelial cell adhesion molecule 1 | 0.323 |
| CLDN1 | claudin 1 | 0.323 |
| PLAU | plasminogen activator, urokinase | 0.323 |
| PRDM1 | PR/SET domain 1 | 0.324 |
| ICAM1 | intercellular adhesion molecule 1 | 0.324 |
| CXCL6 | C-X-C motif chemokine ligand 6 | 0.324 |
| SOCS3 | suppressor of cytokine signaling 3 | 0.324 |
| SLC2A14 | solute carrier family 2 member 14 | 0.324 |
| LAIR2 | leukocyte associated immunoglobulin like receptor 2 | 0.324 |
| GPR97 | | 0.324 |
| CCR1 | C-C motif chemokine receptor 1 | 0.324 |
| TLR2 | toll like receptor 2 | 0.324 |
| C5AR1 | complement C5a receptor 1 | 0.325 |
| PLEK | pleckstrin | 0.325 |
| ANXA5 | annexin A5 | 0.325 |
| CXCR2 | C-X-C motif chemokine receptor 2 | 0.325 |
| BCL6 | BCL6 transcription repressor | 0.325 |
| MMP12 | matrix metallopeptidase 12 | 0.325 |
| EVI2B | ecotropic viral integration site 2B | 0.325 |
| CD55 | CD55 molecule (Cromer blood group) | 0.326 |
| CXCL1 | C-X-C motif chemokine ligand 1 | 0.326 |
| IL1RAP | interleukin 1 receptor accessory protein | 0.327 |
| LILRB3 | leukocyte immunoglobulin like receptor B3 | 0.327 |
| FPR2 | formyl peptide receptor 2 | 0.327 |
| UBASH3B | ubiquitin associated and SH3 domain containing B | 0.327 |
| LPCAT1 | lysophosphatidylcholine acyltransferase 1 | 0.328 |
| FAP | fibroblast activation protein alpha | 0.328 |
| FCGR2C | Fc fragment of IgG receptor IIc (gene/pseudogene) | 0.328 |
| HIF1A | hypoxia inducible factor 1 subunit alpha | 0.329 |
| PXDC1 | PX domain containing 1 | 0.329 |
| HSPA13 | heat shock protein family A (Hsp70) member 13 | 0.330 |
| SLC39A6 | solute carrier family 39 member 6 | 0.330 |
| IFITM3 | interferon induced transmembrane protein 3 | 0.331 |
| F5 | coagulation factor V | 0.331 |
| OSMR | oncostatin M receptor | 0.331 |
| SAMSN1 | SAM domain, SH3 domain and nuclear localization | 0.331 |
| | signals 1 | |
| SELP | selectin P | 0.332 |
| SLC7A11 | solute carrier family 7 member 11 | 0.332 |
| PSAT1 | phosphoserine aminotransferase 1 | 0.335 |
| MMP7 | matrix metallopeptidase 7 | 0.335 |
| SLC9B2 | solute carrier family 9 member B2 | 0.335 |
| NCF2 | neutrophil cytosolic factor 2 | 0.335 |
| CFI | complement factor I | 0.336 |
| CPEB4 | cytoplasmic polyadenylation element binding protein 4 | 0.336 |
| LATS2 | large tumor suppressor kinase 2 | 0.336 |
| SLC7A5 | solute carrier family 7 member 5 | 0.336 |
| CLEC4A | C-type lectin domain family 4 member A | 0.337 |
| FUT8 | fucosyltransferase 8 | 0.337 |
| DYSF | dysferlin | 0.337 |
| SRGN | serglycin | 0.338 |
| ATP11A | ATPase phospholipid transporting 11A | 0.338 |
| ALPL | alkaline phosphatase, biomineralization associated | 0.338 |
| MNDA | myeloid cell nuclear differentiation antigen | 0.338 |
| ANXA1 | annexin A1 | 0.339 |
| KCNJ15 | potassium inwardly rectifying channel subfamily J | 0.339 |
| | member 15 | |
| CREB5 | cAMP responsive element binding protein 5 | 0.339 |
| TBXAS1 | thromboxane A synthase 1 | 0.340 |
| PDE4B | phosphodiesterase 4B | 0.340 |
| PFKFB3 | 6-phosphofructo-2-kinase/fructose-2,6-biphosphatase 3 | 0.340 |
| MMP3 | matrix metallopeptidase 3 | 0.341 |
| TNFRSF10C | TNF receptor superfamily member 10c | 0.342 |
| WISP1 | | 0.342 |
| MXRA5 | matrix remodeling associated 5 | 0.343 |
| LINC00152 | | 0.343 |
| PDPN | podoplanin | 0.343 |
| FCGR3A | Fc fragment of IgG receptor IIIa | 0.343 |
| VNN2 | vanin 2 | 0.344 |
| FPR1 | formyl peptide receptor 1 | 0.344 |
| SERPINE1 | serpin family E member 1 | 0.346 |
| CXCR1 | C-X-C motif chemokine receptor 1 | 0.346 |
| KYNU | kynureninase | 0.347 |
| CLDN2 | claudin 2 | 0.348 |
| MIR4435-1HG | | 0.348 |
| TREM1 | triggering receptor expressed on myeloid cells 1 | 0.348 |
| SERPINA3 | serpin family A member 3 | 0.350 |
| CFB | complement factor B | 0.350 |
| TCN1 | transcobalamin 1 | 0.353 |
| TIMP1 | TIMP metallopeptidase inhibitor 1 | 0.354 |
| FCGR2A | Fc fragment of IgG receptor IIa | 0.354 |
| IL1A | interleukin 1 alpha | 0.355 |
| AQP9 | aquaporin 9 | 0.355 |
| PROK2 | prokineticin 2 | 0.355 |
| ELL2 | elongation factor for RNA polymerase II 2 | 0.356 |
| IFITM2 | interferon induced transmembrane protein 2 | 0.356 |
| LYN | LYN proto-oncogene, Src family tyrosine kinase | 0.356 |
| CSF3R | colony stimulating factor 3 receptor | 0.356 |
| FCGR3B | Fc fragment of IgG receptor IIIb | 0.357 |
| PTAFR | platelet activating factor receptor | 0.359 |
| TRIB2 | tribbles pseudokinase 2 | 0.360 |
| S100A9 | S100 calcium binding protein A9 | 0.363 |
| S100A12 | S100 calcium binding protein A12 | 0.371 |
| S100A8 | S100 calcium binding protein A8 | 0.379 |

TABLE 9
Genes associated with BU and modulated by anti-IL-23p19 antibody treatment

| Symbol | Name | tau |
|---|---|---|
| CCDC175 | coiled-coil domain containing 175 | −0.297 |
| PCK1 | phosphoenolpyruvate carboxykinase 1 | −0.259 |
| CAPN13 | calpain 13 | −0.259 |
| HOXA11-AS | HOXA11 antisense RNA | −0.259 |
| MROH7 | maestro heat like repeat family member 7 | −0.258 |
| SYT7 | synaptotagmin 7 | −0.256 |
| C21orf88 | | −0.255 |
| ANO7 | anoctamin 7 | −0.251 |
| PADI2 | peptidyl arginine deiminase 2 | −0.248 |
| SLC6A7 | solute carrier family 6 member 7 | −0.244 |
| WNK4 | WNK lysine deficient protein kinase 4 | −0.242 |
| LOC101929718 | uncharacterized LOC101929718 | −0.240 |
| PNLIPRP2 | pancreatic lipase related protein 2 (gene/pseudogene) | −0.240 |
| RPL10L | ribosomal protein L10 like | −0.239 |
| EXPH5 | exophilin 5 | −0.238 |
| APBA1 | amyloid beta precursor protein binding family A | −0.236 |
| | member 1 | |
| CCNJL | cyclin J like | −0.232 |
| VIPR1 | vasoactive intestinal peptide receptor 1 | −0.232 |
| PDZD3 | PDZ domain containing 3 | −0.232 |
| CDHR1 | cadherin related family member 1 | −0.231 |
| AUTS2 | activator of transcription and developmental regulator | −0.227 |
| | AUTS2 | |
| CHP2 | calcineurin like EF-hand protein 2 | −0.227 |
| IGSF9 | immunoglobulin superfamily member 9 | −0.226 |
| CA1 | carbonic anhydrase 1 | −0.226 |
| ANP32E | acidic nuclear phosphoprotein 32 family member E | 0.225 |
| JAK2 | Janus kinase 2 | 0.225 |
| IGKV1-8 | immunoglobulin kappa variable 1-8 | 0.225 |
| SSR4 | signal sequence receptor subunit 4 | 0.225 |
| BST2 | bone marrow stromal cell antigen 2 | 0.225 |
| IGKV1-9 | immunoglobulin kappa variable 1-9 | 0.226 |
| FBXO18 | | 0.226 |
| HLA-DPA1 | major histocompatibility complex, class II, DP alpha 1 | 0.226 |
| NCF2 | neutrophil cytosolic factor 2 | 0.226 |
| ATG4C | autophagy related 4C cysteine peptidase | 0.226 |
| DUSP5 | dual specificity phosphatase 5 | 0.226 |
| PXDC1 | PX domain containing 1 | 0.226 |
| TGFBI | transforming growth factor beta induced | 0.226 |
| SERPINE1 | serpin family E member 1 | 0.226 |
| LIMS1 | LIM zinc finger domain containing 1 | 0.226 |
| PRDX1 | peroxiredoxin 1 | 0.226 |
| SEL1L | SEL1L adaptor subunit of ERAD E3 ubiquitin ligase | 0.226 |
| S100A9 | S100 calcium binding protein A9 | 0.227 |
| IL1RAP | interleukin 1 receptor accessory protein | 0.227 |
| ENPP2 | ectonucleotide pyrophosphatase/phosphodiesterase 2 | 0.227 |
| PDE4B | phosphodiesterase 4B | 0.227 |
| PGD | phosphogluconate dehydrogenase | 0.227 |
| IGKV1D-27 | immunoglobulin kappa variable 1D-27 (pseudogene) | 0.227 |
| IGKV1D-39 | immunoglobulin kappa variable 1D-39 | 0.227 |
| TXNDC15 | thioredoxin domain containing 15 | 0.227 |
| TNC | tenascin C | 0.227 |
| ZBP1 | Z-DNA binding protein 1 | 0.227 |
| FAM46C | | 0.227 |
| PCNX | | 0.227 |
| ENTPD1 | ectonucleoside triphosphate diphosphohydrolase 1 | 0.227 |
| RAB8B | RAB8B, member RAS oncogene family | 0.227 |
| LOC440895 | two pore channel 3 pseudogene | 0.227 |
| PDZK1IP1 | PDZK1 interacting protein 1 | 0.227 |
| IGKV1D-37 | immunoglobulin kappa variable 1D-37 (non- | 0.227 |
| | functional) | |
| NLRP14 | NLR family pyrin domain containing 14 | 0.227 |
| CDV3 | CDV3 homolog | 0.227 |
| PDIA4 | protein disulfide isomerase family A member 4 | 0.227 |
| FUT11 | fucosyltransferase 11 | 0.227 |
| C4BPA | complement component 4 binding protein alpha | 0.228 |
| LPCAT1 | lysophosphatidylcholine acyltransferase 1 | 0.228 |
| KIAA0040 | KIAA0040 | 0.228 |
| CHST15 | carbohydrate sulfotransferase 15 | 0.228 |
| CLPTM1L | CLPTM1 like | 0.228 |
| IRAK3 | interleukin 1 receptor associated kinase 3 | 0.228 |
| ATP11A | ATPase phospholipid transporting 11A | 0.228 |
| SLC15A4 | solute carrier family 15 member 4 | 0.228 |
| STOM | stomatin | 0.228 |
| IGKV1-37 | immunoglobulin kappa variable 1-37 (non-functional) | 0.228 |
| PLCG2 | phospholipase C gamma 2 | 0.228 |
| PHLDA1 | pleckstrin homology like domain family A member 1 | 0.228 |
| RNF149 | ring finger protein 149 | 0.228 |
| IDO1 | indoleamine 2,3-dioxygenase 1 | 0.228 |
| SYTL3 | synaptotagmin like 3 | 0.228 |
| GOLT1B | golgi transport 1B | 0.229 |
| IFITM3 | interferon induced transmembrane protein 3 | 0.229 |
| CLEC7A | C-type lectin domain containing 7A | 0.229 |
| PPT1 | palmitoyl-protein thioesterase 1 | 0.229 |
| DYX1C1- | | 0.229 |
| CCPG1 | | |
| ISG20 | interferon stimulated exonuclease gene 20 | 0.229 |
| STRIP2 | striatin interacting protein 2 | 0.229 |
| IGKV1-17 | immunoglobulin kappa variable 1-17 | 0.229 |
| FECH | ferrochelatase | 0.229 |
| GM2A | GM2 ganglioside activator | 0.229 |
| TNFAIP8 | TNF alpha induced protein 8 | 0.229 |
| SLAMF7 | SLAM family member 7 | 0.229 |
| UBD | ubiquitin D | 0.229 |
| RHOQ | ras homolog family member Q | 0.229 |
| CCR1 | C-C motif chemokine receptor 1 | 0.229 |
| IGKV1-5 | immunoglobulin kappa variable 1-5 | 0.230 |
| IGKV1-6 | immunoglobulin kappa variable 1-6 | 0.230 |
| ARNTL2 | aryl hydrocarbon receptor nuclear translocator like 2 | 0.230 |
| BRCA2 | BRCA2 DNA repair associated | 0.230 |
| GBP5 | guanylate binding protein 5 | 0.230 |
| UBE2J1 | ubiquitin conjugating enzyme E2 J1 | 0.230 |
| PLA1A | phospholipase A1 member A | 0.230 |
| AAED1 | | 0.230 |
| KLHL6 | kelch like family member 6 | 0.230 |
| HOMER1 | homer scaffold protein 1 | 0.230 |
| CALU | calumenin | 0.230 |
| SAT1 | spermidine/spermine N1-acetyltransferase 1 | 0.230 |
| SEC24A | SEC24 homolog A, COPII coat complex component | 0.230 |
| BLOC1S5- | BLOC1S5-TXNDC5 readthrough (NMD candidate) | 0.231 |
| TXNDC5 | | |
| PLXNC1 | plexin C1 | 0.231 |
| CYTIP | cytohesin 1 interacting protein | 0.231 |
| FAM114A2 | family with sequence similarity 114 member A2 | 0.231 |
| TXNDC5 | thioredoxin domain containing 5 | 0.231 |
| CEP170 | centrosomal protein 170 | 0.231 |
| TTC9 | tetratricopeptide repeat domain 9 | 0.231 |
| CXorf21 | | 0.231 |
| UBE2L6 | ubiquitin conjugating enzyme E2 L6 | 0.231 |
| XPOT | exportin for tRNA | 0.231 |
| TFEC | transcription factor EC | 0.231 |
| LMAN1 | lectin, mannose binding 1 | 0.231 |
| TNFAIP3 | TNF alpha induced protein 3 | 0.231 |
| MMP12 | matrix metallopeptidase 12 | 0.232 |
| FCGR2A | Fc fragment of IgG receptor IIa | 0.232 |
| IGKV1D-16 | immunoglobulin kappa variable 1D-16 | 0.232 |
| NPL | N-acetylneuraminate pyruvate lyase | 0.232 |
| RIPK2 | receptor interacting serine/threonine kinase 2 | 0.232 |
| LYN | LYN proto-oncogene, Src family tyrosine kinase | 0.232 |
| CYLD | CYLD lysine 63 deubiquitinase | 0.232 |
| IGKV1-27 | immunoglobulin kappa variable 1-27 | 0.232 |
| DAPP1 | dual adaptor of phosphotyrosine and 3- | 0.232 |
| | phosphoinositides 1 | |
| IGKV1D-42 | immunoglobulin kappa variable 1D-42 (non- | 0.232 |
| | functional) | |
| CHSY1 | chondroitin sulfate synthase 1 | 0.232 |
| CKLF | chemokine like factor | 0.233 |
| NCOA7 | nuclear receptor coactivator 7 | 0.233 |
| UBASH3B | ubiquitin associated and SH3 domain containing B | 0.233 |
| CPNE5 | copine 5 | 0.233 |
| IGKV1D-33 | immunoglobulin kappa variable 1D-33 | 0.233 |
| FCGR3B | Fc fragment of IgG receptor IIIb | 0.233 |
| CBFB | core-binding factor subunit beta | 0.233 |
| BLOC1S6 | biogenesis of lysosomal organelles complex 1 subunit 6 | 0.233 |
| SLAMF8 | SLAM family member 8 | 0.233 |
| FCGR3A | Fc fragment of IgG receptor IIIa | 0.233 |
| TK2 | thymidine kinase 2 | 0.233 |
| AGTPBP1 | ATP/GTP binding protein 1 | 0.233 |
| CDC27 | cell division cycle 27 | 0.233 |
| MMP7 | matrix metallopeptidase 7 | 0.233 |
| FPR3 | formyl peptide receptor 3 | 0.233 |
| AQP9 | aquaporin 9 | 0.233 |
| SAA2 | serum amyloid A2 | 0.233 |
| TIMP1 | TIMP metallopeptidase inhibitor 1 | 0.234 |
| MANEA | mannosidase endo-alpha | 0.234 |
| IFNG-AS1 | IFNG antisense RNA 1 | 0.234 |
| IFITM2 | interferon induced transmembrane protein 2 | 0.234 |
| TIFA | TRAF interacting protein with forkhead associated | 0.234 |
| | domain | |
| SEC22C | SEC22 homolog C, vesicle trafficking protein | 0.234 |
| CCDC69 | coiled-coil domain containing 69 | 0.234 |
| IGKC | immunoglobulin kappa constant | 0.234 |
| ARPC1B | actin related protein 2/3 complex subunit 1B | 0.234 |
| BHLHE40 | basic helix-loop-helix family member e40 | 0.234 |
| SPCS2 | signal peptidase complex subunit 2 | 0.234 |
| GALNT2 | polypeptide N-acetylgalactosaminyltransferase 2 | 0.235 |
| CLIP4 | CAP-Gly domain containing linker protein family | 0.235 |
| | member 4 | |
| FAM49A | | 0.235 |
| CTSB | cathepsin B | 0.235 |
| TRAM1 | translocation associated membrane protein 1 | 0.235 |
| HIVEP2 | HIVEP zinc finger 2 | 0.235 |
| TCN1 | transcobalamin 1 | 0.235 |
| SAA1 | serum amyloid A1 | 0.235 |
| ANXA5 | annexin A5 | 0.236 |
| HLA-DOA | major histocompatibility complex, class II, DO alpha | 0.236 |
| CYSLTR1 | cysteinyl leukotriene receptor 1 | 0.236 |
| ARRDC3 | arrestin domain containing 3 | 0.236 |
| SPCS3 | signal peptidase complex subunit 3 | 0.236 |
| MGP | matrix Gla protein | 0.236 |
| TARSL2 | | 0.236 |
| GPR183 | G protein-coupled receptor 183 | 0.236 |
| RECQL | RecQ like helicase | 0.236 |
| CCR5 | C-C motif chemokine receptor 5 | 0.236 |
| LRRK2 | leucine rich repeat kinase 2 | 0.236 |
| CASP4 | caspase 4 | 0.237 |
| TMEM156 | transmembrane protein 156 | 0.237 |
| MLKL | mixed lineage kinase domain like pseudokinase | 0.237 |
| FGFR1OP2 | FGFR1 oncogene partner 2 | 0.237 |
| MDM2 | MDM2 proto-oncogene | 0.237 |
| CDK14 | cyclin dependent kinase 14 | 0.237 |
| CCDC109B | | 0.237 |
| GLA | galactosidase alpha | 0.237 |
| GYG1 | glycogenin 1 | 0.237 |
| IFITM1 | interferon induced transmembrane protein 1 | 0.237 |
| GPR155 | G protein-coupled receptor 155 | 0.237 |
| PFKFB3 | 6-phosphofructo-2-kinase/fructose-2,6-biphosphatase 3 | 0.238 |
| KMO | kynurenine 3-monooxygenase | 0.238 |
| NUGGC | nuclear GTPase, germinal center associated | 0.238 |
| SCRN1 | secernin 1 | 0.238 |
| MIAT | myocardial infarction associated transcript | 0.238 |
| CYBB | cytochrome b-245 beta chain | 0.238 |
| SSR3 | signal sequence receptor subunit 3 | 0.238 |
| MTDH | metadherin | 0.239 |
| ACOXL | acyl-CoA oxidase like | 0.239 |
| P4HA1 | prolyl 4-hydroxylase subunit alpha 1 | 0.239 |
| ACVR1 | activin A receptor type 1 | 0.239 |
| ARFGAP3 | ADP ribosylation factor GTPase activating protein 3 | 0.239 |
| CEP97 | centrosomal protein 97 | 0.239 |
| ZDBF2 | zinc finger DBF-type containing 2 | 0.239 |
| WARS | | 0.239 |
| CYB5R2 | cytochrome b5 reductase 2 | 0.239 |
| OSBPL3 | oxysterol binding protein like 3 | 0.239 |
| IGSF6 | immunoglobulin superfamily member 6 | 0.239 |
| CTSL | cathepsin L | 0.239 |
| FCGR2B | Fc fragment of IgG receptor IIb | 0.239 |
| IL1A | interleukin 1 alpha | 0.239 |
| TLR8 | toll like receptor 8 | 0.240 |
| CTSK | cathepsin K | 0.240 |
| HELB | DNA helicase B | 0.240 |
| TAGAP | T cell activation RhoGTPase activating protein | 0.240 |
| EDEM1 | ER degradation enhancing alpha-mannosidase like | 0.240 |
| | protein 1 | |
| SLA | Src like adaptor | 0.240 |
| ATP10D | ATPase phospholipid transporting 10D (putative) | 0.240 |
| SHCBP1 | SHC binding and spindle associated 1 | 0.240 |
| CD86 | CD86 molecule | 0.240 |
| ADA | adenosine deaminase | 0.240 |
| EVI2A | ecotropic viral integration site 2A | 0.240 |
| SAMD8 | sterile alpha motif domain containing 8 | 0.240 |
| HPS5 | HPS5 biogenesis of lysosomal organelles complex 2 | 0.241 |
| | subunit 2 | |
| SAMHD1 | SAM and HD domain containing deoxynucleoside | 0.241 |
| | triphosphate triphosphohydrolase 1 | |
| DENND5B | DENN domain containing 5B | 0.241 |
| SAMSN1 | SAM domain, SH3 domain and nuclear localization | 0.241 |
| | signals 1 | |
| NARF | nuclear prelamin A recognition factor | 0.241 |
| ERO1L | | 0.241 |
| GPR137B | G protein-coupled receptor 137B | 0.241 |
| E2F5 | E2F transcription factor 5 | 0.241 |
| PANX1 | pannexin 1 | 0.241 |
| LINC00152 | | 0.241 |
| FCRL5 | Fc receptor like 5 | 0.241 |
| SERPINA3 | serpin family A member 3 | 0.242 |
| FAM129A | | 0.242 |
| NXPE3 | neurexophilin and PC-esterase domain family member 3 | 0.242 |
| ELOVL5 | ELOVL fatty acid elongase 5 | 0.242 |
| USP48 | ubiquitin specific peptidase 48 | 0.242 |
| PAPSS1 | 3′-phosphoadenosine 5′-phosphosulfate synthase 1 | 0.242 |
| SLC7A11 | solute carrier family 7 member 11 | 0.242 |
| ANXA1 | annexin A1 | 0.242 |
| DUOXA2 | dual oxidase maturation factor 2 | 0.242 |
| ZFAT | zinc finger and AT-hook domain containing | 0.242 |
| ZNF546 | zinc finger protein 546 | 0.243 |
| SRGN | serglycin | 0.243 |
| PHTF1 | putative homeodomain transcription factor 1 | 0.243 |
| DEGS1 | delta 4-desaturase, sphingolipid 1 | 0.244 |
| APOBEC3G | apolipoprotein B mRNA editing enzyme catalytic | 0.244 |
| | subunit 3G | |
| FCER1G | Fc fragment of IgE receptor Ig | 0.244 |
| SPATS2 | spermatogenesis associated serine rich 2 | 0.244 |
| PLEK | pleckstrin | 0.244 |
| SEC14L1 | SEC14 like lipid binding 1 | 0.244 |
| CD38 | CD38 molecule | 0.245 |
| XBP1 | X-box binding protein 1 | 0.245 |
| TBCEL | tubulin folding cofactor E like | 0.245 |
| SFMBT2 | Scm like with four mbt domains 2 | 0.245 |
| KYNU | kynureninase | 0.245 |
| KCNN3 | potassium calcium-activated channel subfamily N | 0.245 |
| | member 3 | |
| MIR4435-1HG | | 0.246 |
| DNAJC1 | DnaJ heat shock protein family (Hsp40) member C1 | 0.246 |
| SLC7A5 | solute carrier family 7 member 5 | 0.246 |
| BAG2 | BAG cochaperone 2 | 0.247 |
| PRKD3 | protein kinase D3 | 0.247 |
| CLEC4A | C-type lectin domain family 4 member A | 0.247 |
| ANKRD36BP2 | ankyrin repeat domain 36B pseudogene 2 | 0.247 |
| C3AR1 | complement C3a receptor 1 | 0.247 |
| MXRA5 | matrix remodeling associated 5 | 0.248 |
| PRDM1 | PR/SET domain 1 | 0.248 |
| IFI30 | IFI30 lysosomal thiol reductase | 0.248 |
| BCAT1 | branched chain amino acid transaminase 1 | 0.248 |
| CDC25B | cell division cycle 25B | 0.248 |
| PLA2G7 | phospholipase A2 group VII | 0.248 |
| ZNF215 | zinc finger protein 215 | 0.248 |
| GLCCI1 | glucocorticoid induced 1 | 0.248 |
| SGTB | small glutamine rich tetratricopeptide repeat containing | 0.248 |
| | beta | |
| HAVCR2 | hepatitis A virus cellular receptor 2 | 0.248 |
| SCPEP1 | serine carboxypeptidase 1 | 0.249 |
| BNIP3L | BCL2 interacting protein 3 like | 0.249 |
| RHBDD1 | rhomboid domain containing 1 | 0.250 |
| HLA-DRA | major histocompatibility complex, class II, DR alpha | 0.250 |
| GNB5 | G protein subunit beta 5 | 0.250 |
| SERPINI1 | serpin family I member 1 | 0.250 |
| LAP3 | leucine aminopeptidase 3 | 0.251 |
| MAP3K8 | mitogen-activated protein kinase kinase kinase 8 | 0.251 |
| DDIT4 | DNA damage inducible transcript 4 | 0.252 |
| LPIN1 | lipin 1 | 0.252 |
| HLA-DMB | major histocompatibility complex, class II, DM beta | 0.252 |
| ZNF438 | zinc finger protein 438 | 0.253 |
| MB21D1 | | 0.253 |
| LAX1 | lymphocyte transmembrane adaptor 1 | 0.253 |
| HLA-DMA | major histocompatibility complex, class II, DM alpha | 0.254 |
| RFX5 | regulatory factor X5 | 0.254 |
| PDK1 | pyruvate dehydrogenase kinase 1 | 0.254 |
| LATS2 | large tumor suppressor kinase 2 | 0.255 |
| PRDX4 | peroxiredoxin 4 | 0.255 |
| TBXAS1 | thromboxane A synthase 1 | 0.255 |
| EVI2B | ecotropic viral integration site 2B | 0.255 |
| DNAJB9 | DnaJ heat shock protein family (Hsp40) member B9 | 0.255 |
| LRP8 | LDL receptor related protein 8 | 0.256 |
| BNIP3 | BCL2 interacting protein 3 | 0.256 |
| TRIB2 | tribbles pseudokinase 2 | 0.256 |
| EAF2 | ELL associated factor 2 | 0.256 |
| FUT8 | fucosyltransferase 8 | 0.257 |
| SLC39A6 | solute carrier family 39 member 6 | 0.257 |
| MIR3140 | microRNA 3140 | 0.258 |
| CTSH | cathepsin H | 0.259 |
| ELL2 | elongation factor for RNA polymerase II 2 | 0.259 |
| CPEB4 | cytoplasmic polyadenylation element binding protein 4 | 0.260 |
| DRAM1 | DNA damage regulated autophagy modulator 1 | 0.260 |
| SEC11C | SEC11 homolog C, signal peptidase complex subunit | 0.261 |
| LY96 | lymphocyte antigen 96 | 0.262 |
| CLDN2 | claudin 2 | 0.263 |
| HSPA13 | heat shock protein family A (Hsp70) member 13 | 0.263 |
| SNX25 | sorting nexin 25 | 0.266 |
| PSAT1 | phosphoserine aminotransferase 1 | 0.267 |
| IFNAR2 | interferon alpha and beta receptor subunit 2 | 0.267 |
| BIRC3 | baculoviral IAP repeat containing 3 | 0.267 |
| CEP128 | centrosomal protein 128 | 0.268 |
| LIPA | lipase A, lysosomal acid type | 0.269 |
| FBXW7 | F-box and WD repeat domain containing 7 | 0.271 |
| CFB | complement factor B | 0.273 |
| TNFRSF17 | TNF receptor superfamily member 17 | 0.277 |

The results provided in the Examples demonstrated a distinct pattern of transcriptional changes after mirikizumab treatment that correlated with disease activity. The changes mediated by mirikizumab included transcripts that are enriched in TNF^{R} mucosa, providing an opportunity to intervene using mirikizumab at this p19-mediated pathway in such patients. The changes mediated by mirikizumab were robust at week 12 and were maintained through week 52 in patients with ulcerative colitis. Administration of an anti-IL-23p19 antibody normalized genes associated with both stool frequency and bowel urgency.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

### NUMBERED EMBODIMENTS

1. A method of treating ulcerative colitis in a patient having or suspected of having ulcerative colitis, the method comprising: obtaining a first sample from the patient; analyzing the first sample to detect at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; administering an anti-IL-23p19 antibody to the patient; obtaining a second sample from the patient; and analyzing the second sample to detect at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16, wherein a change in expression level of the at least one biomarker detected in the second sample from the expression level of the at least one biomarker detected in the first sample indicates a response to the anti-IL-23p19 antibody.

2. In another aspect, the present disclosure is directed to a method of identifying a patient having or suspected of having anti-Tumor Necrosis Factor (anti-TNF) therapy resistance (anti-TNF^{R}) as a candidate patient for receiving anti-IL-23p19 antibody treatment for ulcerative colitis, the method comprising: obtaining a sample from the patient; analyzing the sample for at least one biomarker of anti-Tumor Necrosis Factor (anti-TNF) therapy resistance (anti-TNF^{R}); and identifying the patient as a candidate patient for receiving the anti-IL-23p19 antibody treatment based on the analysis of the biomarker

3. A method of treating a patient having or suspected of having ulcerative colitis and who has or is suspected of having resistance to anti-TNF treatment with an anti-IL-23p19 antibody, the method comprising: determining if the patient is TNF^{R}; and treating the patient with an anti-IL-23p19 antibody if the patient is TNF^{R}.

4. A method of treating a symptom associated with ulcerative colitis in a patient having or suspected of having ulcerative colitis, the method comprising: obtaining a first sample from the patient; analyzing the sample for at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; administering an anti-IL-23p19 antibody to the patient; obtaining second sample from the patient; and analyzing the second sample for at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16.

5. A method for diagnosing ulcerative colitis in a patient in a patient having or suspected of having ulcerative colitis, the method comprising: (a) determining an expression level of at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16 in a sample obtained from the patient, (b) comparing the determined expression level of the at least one biomarker to a reference expression level of at least one biomarker selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; and (c) providing a diagnosis of ulcerative colitis if the biomarker expression level in the patient is increased as compared to the reference expression level or if the biomarker expression level in the patient is decreased as compared to the reference expression level.

6. A method of diagnosing ulcerative colitis in a patient having or suspected of having ulcerative colitis, the method comprising: (a) using an analyzer unit to determine an expression level of at least one of a biomarker including CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16 in a sample obtained from a patient; (b) using a computing device to compare the determined expression level(s) of the at least one biomarker to a reference expression level of at least one of a biomarker including CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; and (c) providing a diagnosis of ulcerative colitis if the biomarker expression level is increased as compared to the reference expression level or if the biomarker expression level is decreased as compared to the reference expression level.

7. A method of determining whether a patient having or suspected of having ulcerative colitis is healing in response to anti-IL-23p19 antibody treatment, the method comprising: analyzing a sample obtained from a patient before the patient receives anti-IL-23p19 antibody treatment for at least one biomarker including CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; analyzing a sample obtained from a patient after the patient receives the anti-IL-23p19 antibody treatment for at least one biomarker comprising CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; and determining that the patient having or suspected of having ulcerative colitis is healing in response to the anti-IL-23p19 antibody treatment if a change in expression level in the at least one biomarker after the patient receives the anti-IL-23p19 antibody treatment is detected.

8. A biomarker panel comprising at least one biomarker comprising CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16.

9. A method of treating stool frequency in a patient having or suspected of having ulcerative colitis, the method comprising: obtaining a first sample from the patient; analyzing the first sample to detect a biomarker selected from Table 8 and combinations thereof, administering an anti-IL-23p19 antibody to the patient; obtaining a second sample from the patient; and analyzing the second sample to detect a biomarker selected from Table 8 and combinations thereof, wherein a change in expression level of the at least one biomarker detected in the second sample from the expression level of the at least one biomarker detected in the first sample indicates a response to the anti-IL-23p19 antibody.

10. A method for diagnosing stool frequency in a patient in a patient having or suspected of having ulcerative colitis, the method comprising: (a) determining an expression level of a biomarker selected from Table 8 and combinations thereof in a sample obtained from the patient, (b) comparing the determined expression level of the biomarker to a reference expression level of a biomarker selected from Table 8 and combinations thereof; and (c) providing a diagnosis of stool frequency if the biomarker expression level in the patient is changed as compared to the reference expression level.

11. A method of treating bowel urgency in a patient having or suspected of having ulcerative colitis, the method comprising: obtaining a first sample from the patient; analyzing the first sample to detect a biomarker selected from Table 9 and combinations thereof, administering an anti-IL-23p19 antibody to the patient; obtaining a second sample from the patient; and analyzing the second sample to detect a biomarker selected from Table 9 and combinations thereof, wherein a change in expression level of the at least one biomarker detected in the second sample from the expression level of the at least one biomarker detected in the first sample indicates a response to the anti-IL-23p19 antibody.

12. A method of diagnosing bowel urgency in a patient having or suspected of having ulcerative colitis, the method comprising: (a) determining an expression level of a biomarker selected from Table 9 and combinations thereof in a sample obtained from the patient, (b) comparing the determined expression level of the biomarker to a reference expression level of a biomarker selected from Table 9 and combinations thereof, and (c) providing a diagnosis of bowel urgency if the biomarker expression level in the patient is changed as compared to the reference expression level.

13. A method of diagnosing stool frequency in a patient having or suspected of having ulcerative colitis, the method comprising: (a) using an analyzer unit to determine an expression level of a biomarker selected from Table 8 and combinations thereof in a sample obtained from the patient; (b) using a computing device to compare the determined expression level(s) of the biomarker to a reference expression level of a biomarker selected from Table 8 and combinations thereof; and (c) providing a diagnosis of stool frequency if the biomarker expression level is changed as compared to the reference expression level.

14. A method of diagnosing bowel urgency in a patient having or suspected of having ulcerative colitis, the method comprising: (a) using an analyzer unit to determine an expression level of a biomarker selected from Table 9 and combinations thereof in a sample obtained from the patient; (b) using a computing device to compare the determined expression level(s) of the biomarker to a reference expression level of a biomarker selected from Table 9 and combinations thereof; and (c) providing a diagnosis of bowel urgency if the biomarker expression level is changed as compared to the reference expression level.

15. A biomarker panel comprising at least one biomarker selected from Table 8.

16. A biomarker panel comprising at least one biomarker selected from Table 9.

1-265. (canceled)

266. A method of treating ulcerative colitis in a patient in need thereof, wherein the method comprises:
obtaining a first sample from the patient; analyzing the first sample to detect five or more gene transcript biomarker(s) of five or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16;
administering an anti-IL-23p19 antibody to the patient;
obtaining a second sample from the patient; and analyzing the second sample to detect five or more gene transcript biomarker(s) of five or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16,
wherein a change in expression level of the five or more gene transcript biomarker(s) detected in the second sample from the expression level of the five or more gene transcript biomarkers detected in the first sample indicates a response to the anti-IL-23p19 antibody.

267. A method treating ulcerative colitis in a patient in need thereof according to claim 266, wherein the method comprises:
obtaining a first sample from the patient; analyzing the first sample to detect five or more gene transcript biomarker(s) of five or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2 and ADH1C;
administering an anti-IL-23p19 antibody to the patient;
obtaining a second sample from the patient; and analyzing the second sample to detect five or more gene transcript biomarker(s) of five or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2 and ADH1C;
wherein a change in expression level of the five or more gene transcript biomarker(s) detected in the second sample from the expression level of the five or more gene transcript biomarkers detected in the first sample indicates a response to the anti-IL-23p19 antibody.

268. A method of treating ulcerative colitis in a patient in need thereof according to claim 266, wherein the method comprises detecting the expression level of at least six, at least 7, at least 8, at least 9, or at least 10 of the gene transcript biomarker(s) of the genes recited in claim 266 prior to and after administration of the anti-IL-23p19 antibody.

269. A method of treating ulcerative colitis in a patient in need thereof according to claim 266, wherein a change in the expression of the five or more gene transcript biomarkers detected in the second sample from the expression of the five or more gene transcript biomarkers detected in the first sample indicates that administration of the anti-IL-23p19 antibody should be continued.

270. A method of treating ulcerative colitis in a patient in need thereof according to claim 266, wherein five or more gene transcript biomarker(s) is increased following administration of the anti-IL-23p19 antibody, and wherein the five or more gene transcript biomarker(s) are GUCA2B, OTOP2, AQP8, SLC26A2, ADH1C, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4 and ZG16.

271. A method of treating ulcerative colitis in a patient in need thereof according to claim 266, wherein five or more gene transcript biomarker(s) is decreased following the anti-IL-23p19 antibody treatment, and wherein the five or more gene transcript biomarker(s) are CXCL8, AQP9, IL1B, S100A9, TREM1, MMP12, MMP1, MMP7, TCN1, DUOX2, DUOXA2, SLC6A14, VNN1, ABCA12, REG1B, C4BPA, REG3A, DMBT1, REG1P, S100A8, IGKV2D-40, P13, TNIP3, REG1A, IDO1, NOS2, MMP10, CXCL1 and PTGS2.

272. A method of treating ulcerative colitis in a patient in need thereof according to claim 266, wherein the expression level of the five of more gene transcript biomarker(s) is determined by a method of gene expression profiling.

273. A method of treating ulcerative colitis in a patient in need thereof according to claim 272, wherein the method of gene expression profiling is a PCR-based method, immunohistochemistry and/or proteomics technology.

274. A method of treating ulcerative colitis in a patient in need thereof according to claim 272, wherein said expression levels of the five or more gene transcript biomarker(s) are normalized relative to the expression levels of five or more reference genes, or their expression products.

275. A method of treating ulcerative colitis in a patient in need thereof according claim 266, wherein the sample is from a colonic tissue biopsy or rectal tissue biopsy.

276. A method of treating ulcerative colitis in a patient in need thereof according to claim 275, wherein the colonic tissue biopsy is from a tissue selected from the group consisting of the terminal ileum, the ascending colon, the descending colon, and the sigmoid colon.

277. A method of treating ulcerative colitis in a patient in need thereof according to claim 275, wherein the colonic tissue biopsy is from a non-inflamed colonic area and/or wherein the colonic tissue biopsy is from an inflamed colonic area.

278. A method of treating ulcerative colitis in a patient in need thereof according to claim 266, wherein the first sample is taken before or simultaneous with administration of the anti-IL-23p19 antibody and wherein the second sample is taken at least two weeks, at least four weeks, at least eight weeks, at least twelve weeks, at least sixteen weeks, at least twenty weeks, at least twenty-four weeks, at least twenty-eight weeks, at least thirty weeks, at least thirty-two weeks, at least thirty-six weeks, at least forty weeks, at least forty-four weeks, at least forty-eight weeks, or at least fifty-two weeks, after the first administration of the anti-IL-23p19 antibody.

279. A method of treating ulcerative colitis in a patient in need thereof according to claim 266, wherein the anti-IL-23p19 antibody is mirikizumab, guselkumab, risankizumab, tildrakizumab or brazikumab.

280. A method of treating ulcerative colitis in a patient in need thereof according to claim 279, wherein the method comprises:
a) administering three induction doses of mirikizumab to the patient by intravenous infusion at 4-week intervals, wherein each induction dose comprises 300 mg of mirikizumab; and
b) administering multiple maintenance doses of mirikizumab to the patient by subcutaneous injection at 4 week intervals, wherein the first maintenance dose is administered 2-8 weeks after the last induction dose is administered and wherein each maintenance dose comprises 200 mg of mirikizumab.

281. A method of treating a patient having ulcerative colitis and who has or is suspected of having anti-Tumor Necrosis Factor (anti-TNF) therapy resistance (anti-TNF^{R}) with an anti-IL-23p19 antibody, wherein the method comprises:
determining if the patient is anti-TNF^{R} by obtaining a sample from the patient and analyzing the sample for at least one, at least two, at least three, at least four, or at least five or more anti-Tumor Necrosis Factor (anti-TNF) therapy resistance (anti-TNF^{R}) gene transcript biomarker(s) of at least one, at least two, at least three, at least four, or at least five or more genes selected from OSMR, FCGR3, CXCL6, interleukin-11, interleukin-24, interleukin-13RA2, FAP, TWIST1, and WNT2,
and treating the patient with an anti-IL-23p19 antibody if the patient is anti-TNF^{R}.

282. A method of treating a patient having ulcerative colitis and who has or is suspected of having anti-Tumor Necrosis Factor (anti-TNF) therapy resistance (anti-TNF^{R}) with an anti-IL-23p19 antibody according to claim 281, wherein the method further comprises analyzing samples obtained before anti-IL-23p19 antibody administration and following anti-IL-23p19 antibody administration for at least three, at least four, at least five, at least six, at least seven, at least eight, at least nine, or at least ten or more gene transcript biomarker(s) of three or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16,
wherein a change in expression level of the three or more gene transcript biomarker(s) detected in the sample obtained after administration of the anti-IL-23p19 antibody from the expression level of the three or more gene transcript biomarkers detected in the sample obtained prior to administration of the anti-IL-23p19 antibody indicates a response to the anti-IL-23p19 antibody in the anti-TNF^{R} patient.

283. A method of treating a patient having ulcerative colitis and who has or is suspected of having anti-Tumor Necrosis Factor (anti-TNF) therapy resistance (anti-TNF^{R}) with an anti-IL-23p19 antibody according to claim 282, wherein a change in the expression of the at least three or more gene transcript biomarkers detected in the sample taken after administration of the anti-IL-23p19 antibody from the expression of the at least three or more gene transcript biomarkers detected in the sample taken prior to administration of the anti-IL-23p19 antibody indicates that administration of the anti-IL-23p19 antibody to the anti-TNF^{R} patient should be continued.

284. A method of treating a patient having ulcerative colitis and who has or is suspected of having anti-Tumor Necrosis Factor (anti-TNF) therapy resistance (anti-TNF^{R}) with an anti-IL-23p19 antibody according to claim 282, wherein three or more gene transcript biomarker(s) is increased following administration of the anti-IL-23p19 antibody, and wherein the three or more gene transcript biomarker(s) are GUCA2B, OTOP2, AQP8, SLC26A2, ADH1C, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4 and ZG16.

285. A method of treating a patient having ulcerative colitis and who has or is suspected of having anti-Tumor Necrosis Factor (anti-TNF) therapy resistance (anti-TNF^{R}) with an anti-IL-23p19 antibody according to claim 282, wherein three or more gene transcript biomarker(s) is decreased following the anti-IL-23p19 antibody treatment, and wherein the three or more gene transcript biomarker(s) are CXCL8, AQP9, IL1B, S100A9, TREM1, MMP12, MMP1, MMP7, TCN1, DUOX2, DUOXA2, SLC6A14, VNN1, ABCA12, REG1B, C4BPA, REG3A, DMBT1, REG1P, S100A8, IGKV2D-40, P13, TNIP3, REG1A, IDO1, NOS2, MMP10, CXCL1 and PTGS2.

286. A method of treating a patient having ulcerative colitis and who has or is suspected of having anti-Tumor Necrosis Factor (anti-TNF) therapy resistance (anti-TNF^{R}) with an anti-IL-23p19 antibody according to claim 281, wherein the anti-IL-23p19 antibody is mirikizumab, guselkumab, risankizumab, tildrakizumab or brazikumab.

287. A method of determining whether a patient having ulcerative colitis is healing in response to treatment with an anti-IL-23p19 antibody, wherein the method comprises:
(a)(i) analyzing a sample obtained from a patient before the patient receives anti-IL-23p19 antibody treatment for five or more gene transcript biomarker(s) of five or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, P13, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16;
(b)(i) analyzing a sample obtained from a patient after the patient receives the anti-IL-23p19 antibody treatment for five or more gene transcript biomarker(s) of five or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, P13, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; and
(c)(i) determining that the patient having ulcerative colitis is healing in response to the anti-IL-23p19 antibody treatment if a change in expression level in the five or more gene transcript biomarker(s) after the patient receives the anti-IL-23p19 antibody treatment is detected or
(a)(ii) analyzing a sample obtained from a patient having ulcerative colitis who did not receive anti-IL-23p19 antibody treatment for five or more gene transcript biomarker(s) of five or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16;
(b)(ii) analyzing a sample obtained from a patient after the patient receives the anti-IL-23p19 antibody treatment for five or more gene transcript biomarker(s) of five or more genes selected from CXCL8, AQP9, IL1B, S100A9, TREM1, MMP12, MMP1, MMP7, TCN1, DUOX2, DUOXA2, SLC6A14, VNN1, ABCA12, REG1B, C4BPA, GUCA2B, OTOP2, AQP8, SLC26A2, ADH1C, MMP3, REG3A, DMBT1, REG1P, S100A8, IGKV2D-40, PI3, TNIP3, REG1A, IDO1, NOS2, MMP10, CXCL1, PTGS2, ABCG2, HMGCS2, TMIGD1, GUCA2A, LOC101928405, MS4A12, UGT2A3, TRPM6, NXPE4, SLC16A9, ADH1C, PCK1, CDKN2B-AS1, TMEM236, CD177P1, SLC17A4, and ZG16; and
(c)(ii) determining that the patient having ulcerative colitis is healing in response to the anti-IL-23p19 antibody treatment if a change in expression level in the five or more gene transcript biomarker(s) after the patient receives the anti-IL-23p19 antibody treatment is detected as compared to the expression level of the five or more gene transcript biomarker(s) in the patient who did not receive anti-IL-23p19 antibody treatment.

288. A method of determining whether a patient having ulcerative colitis is healing in response to treatment with an anti-IL-23p19 antibody according to claim 287, wherein the method comprises:
(a)(i) analyzing a sample obtained from a patient before the patient receives anti-IL-23p19 antibody treatment for one or more gene transcript biomarker(s) of one or more genes selected from GUCA2A, OTOP2, AQP8, SLC26A2, and ADH1C,
(b)(i) analyzing a sample obtained from a patient after the patient receives the anti-IL-23p19 antibody treatment for one or more gene transcript biomarker(s) of one or more genes selected from GUCA2A, OTOP2, AQP8, SLC26A2, and ADH1C; and
(c)(i) determining that the patient having ulcerative colitis is healing in response to the anti-IL-23p19 antibody treatment if the expression level in the one or more gene transcript biomarker(s) is increased after the patient receives the anti-IL-23p19 antibody treatment or
(a)(ii) analyzing a sample obtained from a patient having ulcerative colitis who did not receive anti-IL-23p19 antibody treatment for one or more gene transcript biomarker(s) of one or more genes selected from GUCA2A, OTOP2, AQP8, SLC26A2, and ADH1C;
(b)(ii) analyzing a sample obtained from a patient after the patient receives the anti-IL-23p19 antibody treatment for one or more gene transcript biomarker(s) of one or more genes selected from GUCA2A, OTOP2, AQP8, SLC26A2, and ADH1C; and
(c)(ii) determining that the patient having ulcerative colitis is healing in response to the anti-IL-23p19 antibody treatment if the expression level in the one or more gene transcript biomarker(s) is increased after the patient receives the anti-IL-23p19 antibody treatment as compared to the expression level of the one or more gene transcript biomarker(s) in the patient who did not receive anti-IL-23p19 antibody treatment.

289. A method of determining whether a patient having ulcerative colitis is healing in response to treatment with an anti-IL-23p19 antibody according to claim 288, wherein the method comprises detecting the expression level at least two, at least three, at least four, or at least five of the gene transcript biomarkers of the genes recited in claim 288.

290. A method of determining whether a patient having ulcerative colitis is healing in response to treatment with an anti-IL-23p19 antibody according to claim 287, wherein the method comprises detecting the expression level at least six, at least seven, at least eight, at least nine, or at least ten of the gene transcript biomarkers of the genes recited in claim 287.

291. A method of treating stool frequency in a patient having ulcerative colitis in a patient in need thereof, wherein the method comprises:
obtaining a first sample from the patient;
analyzing the first sample to detect five or more gene transcript biomarker(s) of genes selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, and Creatine kinase B,
administering an anti-IL-23p19 antibody to the patient;
obtaining a second sample from the patient; and
analyzing the second sample to detect five or more gene transcript biomarker(s) of five or more genes selected from S100 calcium binding protein 8, S100 calcium binding protein A12, Cadherin related family member 1, S100 calcium binding protein A9, Tribbles pseudokinase 2, Platelet activating factor receptor, Apoptosis inducing factor mitochondria associated 3, Fc fragment of IgG receptor IIb, Colony stimulating factor 3 receptor, LYN proto-oncogene, Src family tyrosine kinase, Interferon induced transmembrane protein 2, Calpain 13, Elongation factor for RNA polymerase II 2, Prokineficin 2, Aquaporin 9, Interleukin 1 alpha, Fc fragment of IgG receptor IIa, TIMP metallopeptidase inhibitor 1, Transcobalamin 1, and Creatine kinase B,
wherein a change in expression level of the five or more gene transcript biomarker(s) detected in the second sample from the expression level of the five or more gene transcript biomarker(s) detected in the first sample indicates a response to the anti-IL-23p19 antibody.

292. A method of treating stool frequency in a patient having ulcerative colitis according to claim 291, wherein the method comprises detecting the expression level of at least six, at least seven, at least eight, at least nine, at least ten of the gene transcript biomarkers of the genes recited in claim 291 prior to and after administration of the anti-IL-23p19 antibody.

293. A method of treating stool frequency in a patient having ulcerative colitis according to claim 291, wherein a change in the expression of the five or more gene transcript biomarkers detected in the second sample from the expression of the five or more gene transcript biomarkers detected in the first sample indicates that administration of the anti-IL-23p19 antibody should be continued.

294. A method of treating stool frequency in a patient having ulcerative colitis according to any one of claim 291, wherein the anti-IL-23p19 antibody is mirikizumab, guselkumab, risankizumab, tildrakizumab or brazikumab.

295. A method of treating bowel urgency in a patient having ulcerative colitis, the method comprising:
(a) obtaining a first sample from the patient;
(b) analyzing the first sample to detect five or more gene transcript biomarker(s) of five or more genes selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, and Calpain 13;
(c) administering an anti-IL-23p19 antibody to the patient;
(d) obtaining a second sample from the patient;
(e) analyzing the second sample to detect five or more gene transcript biomarker(s) of five or more genes from five or more gene transcript biomarker(s) of five or more genes selected from Coiled-coil domain containing 175, TNF receptor superfamily member 17, Complement factor B, F-box and WD repeat domain containing 7, Lipase A, lysosomal acid type, Centrosomal protein 128, Baculoviral IAP repeat containing 3, Interferon alpha and beta receptor subunit 2, Phosphoserine aminotransferase 1, Sortin nexin 25, Heat shock protein family A (Hsp70) member 13, Claudin 2, Lymphocyte antigen 96, SEC11 homolog C, signal peptidase complex subunit, DNA damage regulated autophagy modulator 1, Cytoplasmic polyadenylation element binding protein 4, Phosphoenolpyruvate carboxykinase 1, Elongation factor for RNA polymerase II 2, Cathepsin H, and Calpain 13;
wherein a change in expression level of the five or more gene transcript biomarker(s) detected in the second sample from the expression level of the five or more gene transcript biomarker(s) detected in the first sample indicates a response to the anti-IL-23p19 antibody.

296. A method of treating bowel urgency in a patient having ulcerative colitis according to claim 295, wherein the method comprises detecting the expression level of at least six, at least seven, at least eight, at least nine, or at least ten of the gene transcript biomarkers of the genes recited in claim 295 prior to and after administration of the anti-IL-23p19 antibody.

297. A method of treating bowel urgency in a patient having ulcerative colitis according to claim 296, wherein the anti-IL-23p19 antibody is mirikizumab, guselkumab, risankizumab, tildrakizumab or brazikumab.
